(19) United States
(12) Patent Application Publication
(10) Pub. No.: US 2023/0313305 A1
(43) Pub. Date: Oct. 5, 2023

(54) METHODS AND USES OF INFLAMMATORY BOWEL DISEASE BIOMARKERS

(71) Applicant: Washington University, St. Louis, MO (US)

(72) Inventors: Thaddeus Stappenbeck, St. Louis, MO (US); Gerard Kaiko, St. Louis, MO (US); Ta-Chiang Liu, St. Louis, MO (US)

(21) Appl. No.: 18/185,154

(22) Filed: Mar. 16, 2023

Related U.S. Application Data
(63) Continuation of application No. 16/631,980, filed on Jan. 17, 2020, now abandoned.
(60) Provisional application No. 62/533,982, filed on Jul. 18, 2017.

(30) Foreign Application Priority Data
Jul. 18, 2018 (WO) PCT/US2018/042761

(51) Int. Cl. C12Q 1/6883; C12Q 1/686

(52) U.S. Cl. CPC C12Q 1/6883; C12Q 1/686; C12Q 2600/158; G01N 2333/8132; G01N 2800/52; C12Q 2531/113; G01N 2333/521; G01N 2800/065

(57) ABSTRACT

Among the various aspects of the present disclosure is the provision of methods of diagnosing and treating inflammatory bowel disease (IBD) including ulcerative colitis (UC) or Crohn's disease (CD). In particular, the present disclosure provides in part a panel of IBD biomarkers useful in diagnosing and making treatment decisions. In addition, the present disclosure provides methods of treating IBD with a plasminogen activator inhibitor-1(PAI-1) inhibitor or tissue plasminogen activator (tPA).

### CROSS REFERENCE TO RELATED APPLICATIONS

This application is a continuation of U.S. patent application Ser. No. 16/631,980, filed Jan. 17, 2020, which is a § 371 National Entry application of PCT/US2018/042761, filed Jul. 18, 2018, which claims the benefit of U.S. Provisional Application No. 62/533,982, filed Jul. 18, 2017, the disclosures of which is hereby incorporated by reference in its entirety.

### FIELD OF THE INVENTION

The present disclosure generally relates to methods and uses of markers of inflammatory bowel disease activity for diagnosis, prognosis, or treatment of disease.

### BACKGROUND

Inflammatory Bowel Disease (IBD) is a collective designation of chronic diseases which cause inflammation of unknown cause in a gastrointestinal tract, and is a refractory disease of unknown cause with long-lasting diarrhea and hematochezia, including ulcerative colitis and Crohn disease. In contrast to general food poisoning, its medical condition is long-lasting and repeatedly relieved and exacerbated.

Therapy of inflammatory bowel disease includes nutrition therapy, medical therapy, surgery treatment and granulocyte apheresis whereby granulocytes recruited to an inflamed site are selectively removed, or the like. In medical therapy, salazosulfapyridine, 5-aminosalicylic acid (mesalazine type formulation), a steroidal anti-inflammatory agent, an immunosuppressant or the like is used. There is, however, a problem of side effects, such as headache and gastritis caused by sulfapyridine as a metabolite for salazosulfapyridine and infection and adrenal cortex insufficiency caused by excessive immunodepressive effect for a steroidal anti-inflammatory agent.

Expensive biologics are being approved to treat moderate to severe IBD, however it is not currently known who should be treated with which one. Current markers of IBD activity used for diagnosis and prognosis of disease are inadequate (including fecal calprotectin which is widely used). This is in large part because the disease is heterogeneous and it has been challenging to identify a biomarker that is downstream of all the key pro-inflammatory pathways that are variably enhanced in IBD patients.

Therefore, what is needed is a biomarker signature of IBD activity to guide diagnosis, prognosis and treatment.

### BRIEF DESCRIPTION OF THE FIGURES

The application file contains at least one drawing executed in color. Copies of this patent application publication with color drawing(s) will be provided by the Office upon request and payment of the necessary fee. Those of skill in the art will understand that the drawings, described below, are for illustrative purposes only. The drawings are not intended to limit the scope of the present teachings in any way.

FIG. 1 shows a model for an in vitro culture system (Kaiko G and Ryu S et al, Cell, 2016).

FIG. 2 depicts a PCA plot showing IL-17 had subtle effect relative to stem cell differentiation.

FIG. 3 depicts the identification of gene candidates downstream of IL-17A in the epithelium.

FIG. 4 shows the identification of genes with conserved dysregulation amongst IBD patients.

FIG. 5 shows qPCR verification and dose curve: colon.

FIG. 6 depicts the dose curve for the Ileum.

FIG. 7 depicts a textbook view of tPA and its inhibitor PAI-1.

FIG. 8 shows the plasminogen mediated pathway hypothesis.

FIG. 9A, FIG. 9B, FIG. 9C, FIG. 9D and FIG. 9E show tPA is induced by inflammation and derived from epithelial and non-epithelial cells in the mouse. FIG. 9A shows Naïve tissue. FIG. 9B shows DSS epithelial ulcers. FIG. 9C shows DSS adjacent inflamed areas. FIG. 9D shows mock infection. FIG. 9E shows day 10 post infection.

FIG. 10A and FIG. 10B show tPA is induced by inflammation and derived from epithelial and non-epithelial cells in the mouse. No tPA in any CRF het at day 14 and none in any mice at day 0 in the ileum. FIG. 10A shows II-10R2+/−control+ at day 14 post infection. FIG. 10B shows dnKO at day 14 post infection.

FIG. 11 shows tPA is low to absent without inflammation and derived from epithelial and non-epithelial cells in the mouse.

FIG. 12A and FIG. 12B show data which suggests tPA protects against colitis.

FIG. 13A, FIG. 13B and FIG. 13C depict a novel PAI-1 inhibitor elevates tPA levels in the blood and colon. FIG. 13A shows active and total tPA in plasma. FIG. 13B shows active and total tPA in colon. FIG. 13C shows the ratio of active to total tPA in plasma and colon.

FIG. 14A and FIG. 14B show targeting PAI-1 as a therapy (not prophylaxis) in DSS colitis suppresses disease. FIG. 14A shows the percent weight change with control and PAI-1 inhibitor. FIG. 14B shows the colon length in control treated and PAI-1 treated conditions.

FIG. 15A, FIG. 15B, FIG. 15C, FIG. 15D, FIG. 15E, FIG. 15F and FIG. 15G show targeting PAI-1 as a therapy (not prophylaxis) in DSS colitis suppresses disease. FIG. 15A shows the stool consistency score in control and PAI-1 inhibitor treated subjects. FIG. 15B shows the stool blood score in control and PAI-1 inhibitor treated subjects. FIG. 15C shows H&E staining of control subjects. FIG. 15D shows H&E staining in PAI-1 inhibitor treated subjects. FIG. 15E shows the percent length of colon with normal epithelium/goblet cells in control and PAI-1 inhibitor treated subjects. FIG. 15F shows hyperplasia crypt height in control and PAI-1 inhibitor treated subjects. FIG. 15G shows the average muscle thickness in control and PAI-1 inhibitor treated subjects.

FIG. 16A, FIG. 16B and FIG. 16C show PAI-1 inhibition suppresses neutrophil influx. FIG. 16A shows inflamed tissue adjacent to ulcer in control treated subjects. FIG. 16B shows inflamed tissue adjacent to ulcer in PAI-1 inhibitor treated subjects. FIG. 16C shows the number of Ly6G+ neutrophils per high-power field in control and PAI-1 inhibitor treated subjects.

FIG. 17 shows PAI-1 inhibition suppresses IL-6.

FIG. 18A and FIG. 18B depict a trend to reduced weight loss and bacterial burden with PAI-1 inhibition. FIG. 18A show the percent weight change in control and PAI-1 inhibitor treated subjects. FIG. 18B shows the CFU per gram of feces in control and PAI-1 inhibitor treated subjects.

FIG. 19A, FIG. 19B, FIG. 19C, FIG. 19D and FIG. 19E show PAI-1 inhibition suppresses crypt hyperplasia. FIG. 19A and FIG. 19B depict H&E staining of control treated subjects. FIG. 19C and FIG. 19D depict H&E staining of PAI-1 inhibitor treated subjects. FIG. 19E shows the hyperplastic crypt height in control and PAI-1 inhibitor treated subjects.

FIG. 20A, FIG. 20B and FIG. 20C show PAI-1 inhibition suppresses IL-6, MPO activity and Ly6G+ neutrophils. FIG. 20A shows the amount IL-6 in the colon of control and PAI-1 inhibitor treated subjects. FIG. 20B shows the amount of MPO activity in control and PAI-1 inhibitor treated subjects. FIG. 20C shows the number of Ly6G+ neutrophils per high-power field in control and PAI-1 inhibitor treated subjects.

FIG. 21A shows a schematic of IL-17RA signaling. FIG. 21B shows the fold change of Plat in control, IL-17A, IL=17a+NFKBi, IL=17A+p38i, and IL-17A+CEBPi treated subjects.

FIG. 22A and FIG. 22B shows evidence suggests tPA can directly and indirectly cleave latent TGFβ in cell free assay.

FIG. 23 depicts a schematic of TGF-β pathway. In cancer cell lines most highly upregulated gene is serpin1/PAI-1.

FIG. 24 shows construction of a TGFβ-Smad-luciferase reporter.

FIG. 25 shows TGFβ drives serpine1/PAI-1 expression in the colon spheroids (negative feedback loop).

FIG. 26 shows IL-17A is induced to combat infection/maintain barrier to commensals IT ALSO limits tissue damage through tPA. Perhaps increased PAI-1 in IBD patients limits tissue protective function of IL-17A-tPA. 2. PAI-1 long known to be the most TGFβ-responsive gene may operate as a negative feedback regulator of TGF through tPA. Perhaps dysregulated PAI-1 in IBD explains their hyper-inflammatory state.

FIG. 27 shows tPA is not altered in UC patients, IF staining of sections from surgical resection cases. Therefore, tPA is not a biomarker.

FIG. 28 shows Serpine1/PAI-1 highly up-regulated in inflamed tissue from CD and UC patients (4 cohorts) analysis of deposited raw data in GEO NCBI.

FIG. 29 shows PAI-1 protein highly up-regulated in inflamed tissue from UC patients, IF staining of sections from surgical resection cases.

FIG. 30A and FIG. 30B shows responder v. non-responder subject data. FIG. 30A shows responders vs. non-responders before vedolisumab and infliximab. FIG. 30B shows before infliximab responder vs. non-responder in CD colon and UC colon.

FIG. 31 shows responder v. non-responder subject data.

FIG. 32 shows a graph of responder and non-responder data using PAI as an indicator.

FIG. 33A shows a positive correlation between PAI-1 and IL-6. FIG. 33B shows a positive correlation between PAI-1 and TNF-α.

FIG. 34A shows a positive correlation between PAI-1 and Oncostatin M. FIG. 34B shows a positive correlation between PAI-1 and Ptgs2.

FIG. 35 shows conserved response predicted downstream of IL-17A and IBD.

FIG. 36A shows IPA comparative pathway analysis top 10 overlapping pathways of UC/CD and IL-17A treatment in vitro. FIG. 36B shows acute phase response pathway.

FIG. 37 shows the combined all datasets 2 biomarker signature before infliximab.

FIG. 38 shows the sensitivity versus specificity of the overlapping 5 genes.

FIG. 39A, FIG. 39B, FIG. 39C, FIG. 39D, FIG. 39E and FIG. 39F show a principal components (PC) analysis plots the density of the first three PCs (PC1, PC2, PC3) at diagonal and pairwise scatter plots between them. The black, red and green colored points indicate individual patient samples from cohort 1, 2, 3 respectively. Cohort 3 samples mingled well with cohort 1 & 2 samples based on the first 3 PCs.

FIG. 40 shows the multi-dimensional scaling (MDS, a dimension reduction technique similar to PCA) plot is used to visualize the proximity of the samples on original high dimension on a 2-dimensional plane (MDS dimension 1 vs. MDS dimension 2) with the non-responders in black circle and the responders in green triangle.

FIG. 41A, FIG. 41B, FIG. 41C, FIG. 41D, FIG. 41E, FIG. 41F, FIG. 41G, FIG. 41H, FIG. 41I, FIG. 41J, FIG. 41K, FIG. 41L, FIG. 41M, FIG. 41N, FIG. 41O, FIG. 41P, FIG. 41Q, FIG. 41R, FIG. 41S, FIG. 41T, FIG. 41U, FIG. 41V, FIG. 41W, FIG. 41X, FIG. 41Y, FIG. 41Z and FIG. 41ZA show ROC plots of a portion of the top 100 genes are drawn with the optimal cutoff points. FIG. 41A shows PRNP. FIG. 41B shows ILR13RA2. FIG. 41C shows KLHL5. FIG. 41D shows PTX3. FIG. 41E shows GPX8. FIG. 41F shows IKBIP. FIG. 41G showsTXNDC15. FIG. 41H shows LY96. FIG. 41I shows RNF144B. FIG. 41J shows PDE4B. FIG. 41K shows C1S. FIG. 41L showsST8SIA4. FIG. 41M shows EDNRB. FIG. 41N shows ENTPD1. FIG. 41O shows WNT5A. FIG. 41P shows SAMSN1. FIG. 41Q shows MTMR11. FIG. 41R shows TLR1. FIG. 41S shows MME. FIG. 41T shows CACFD1. FIG. 41U shows CD69. FIG. 41V shows SNAPC1. FIG. 41W shows PRICKLE2. FIG. 41X shows SLAMF7. FIG. 41Y shows TSPAN2. FIG. 41Z shows CXCL6. FIG. 41ZA shows TNFRSF11B

FIG. 42A, FIG. 42B, FIG. 42C, FIG. 42D, FIG. 42E, FIG. 42F, FIG. 42G, FIG. 42H, FIG. 42I, FIG. 42J, FIG. 42K, FIG. 42L, FIG. 42M, FIG. 42N, FIG. 42O, FIG. 42P, FIG. 42Q, FIG. 42R, FIG. 42S, FIG. 42T, FIG. 42U, FIG. 42V, FIG. 42W, FIG. 42X, FIG. 42Y and FIG. 42Z show ROC plots of a portion of the top 100 genes are drawn with the optimal cutoff points. FIG. 42A shows ACSL4. FIG. 42B shows CSGALNACT2. FIG. 42C shows DRAM1. FIG. 42D shows LILRB2. FIG. 42E shows PAPPA. FIG. 42F shows AKR1B1. FIG. 42G shows GPR183. FIG. 42H shows SGTB. FIG. 42I shows GLIPR1. FIG. 42J shows PDPN. FIG. 42K shows RBMS1. FIG. 42L shows SMARCA1. FIG. 42M shows ANGPT2. FIG. 42N shows PLAU. FIG. 42O shows TMEM55A. FIG. 42P shows IGFBP5. FIG. 42Q shows ASAP1. FIG. 42R shows SGCE. FIG. 42S shows HGF. FIG. 42T shows CEBPB. FIG. 42U shows DCBLD1. FIG. 42V shows MCTP1. FIG. 42W shows STAT4. FIG. 42X shows ROBO1. FIG. 42Y shows ARL13B. FIG. 42Z shows AAED1.

FIG. 43A, FIG. 43B, FIG. 43C, FIG. 43D, FIG. 43E, FIG. 43F, FIG. 43G, FIG. 43H, FIG. 43I, FIG. 43J, FIG. 43K, FIG. 43L, FIG. 43M, FIG. 43N, FIG. 43O, FIG. 43P, FIG. 43Q, FIG. 43R, FIG. 43S, FIG. 43T, FIG. 43U, FIG. 43V, FIG. 43W, FIG. 43X, FIG. 43Y, FIG. 42Z and FIG. 43ZA show ROC plots of a portion of the top 100 genes are drawn with the optimal cutoff points. FIG. 43A shows RGS5. FIG. 43B shows TOR1AIP1. FIG. 43C shows CCL18. FIG. 43D shows FERMT2. FIG. 43E shows BPGM. FIG. 43F shows NR3C1. FIG. 43G shows QKI. FIG. 43H shows STX11. FIG. 43I shows DEGS1. FIG. 43J shows THBD. FIG. 43K shows CCL2. FIG. 43L shows HS3ST3B1. FIG. 43M shows SDC2. FIG. 43N shows SLC16A10. FIG. 43O shows VCAN. FIG. 43P shows PXDN. FIG. 43Q shows SRGN. FIG. 43R shows DSE. FIG. 43S shows CAV1. FIG. 43T shows FGFR3. FIG. 43U shows ANGPTL2. FIG. 43V shows CLEC2B. FIG. 43W shows IL7R. FIG. 43X shows CCR1. FIG. 43Y shows LAMC1. FIG. 43Z shows LOX. FIG. 43ZA shows CFL2.

FIG. 44A, FIG. 44B, FIG. 44C, FIG. 44D, FIG. 44E, FIG. 44F, FIG. 44G, FIG. 44H, FIG. 44I, FIG. 44J, FIG. 44K, FIG. 44L, FIG. 44M, FIG. 44N, FIG. 44O, FIG. 44P, FIG. 44Q, FIG. 44R, FIG. 44S and FIG. 44T show ROC plots of a portion of the top 100 genes are drawn with the optimal cutoff points. FIG. 44A shows RDX. FIG. 44B shows SERPINE1. FIG. 44C shows CLIC2. FIG. 44D shows CLMP. FIG. 44E shows SNX10. FIG. 44F shows TNC. FIG. 44G shows FAM49A. FIG. 44H shows S100A9. FIG. 44I shows STC1. FIG. 44J shows ZNF57. FIG. 44K shows PPT1. FIG. 44L shows CYTIP. FIG. 44M shows CTSL. FIG. 44N shows GNB4. FIG. 44O shows LDLRAD3. FIG. 44P shows RGS18. FIG. 44Q shows THEMIS2. FIG. 44R shows BICC1. FIG. 44S shows HSPA13. FIG. 44T shows IL10RA.

FIG. 45A, FIG. 45B, FIG. 45C, FIG. 45D, FIG. 45E, FIG. 45F, FIG. 45G, FIG. 45H, FIG. 45I, FIG. 45J, FIG. 45K, FIG. 45L, FIG. 45M, FIG. 45N, FIG. 45O, FIG. 45P, FIG. 45Q, FIG. 45R, FIG. 45S, FIG. 45T, FIG. 45U, FIG. 45V, FIG. 45W, FIG. 45X, FIG. 45Y and FIG. 45Z show the corresponding sensitivity and specificity at the cutoff point for a portion of the top 100 genes. FIG. 45A shows PRNP. FIG. 45B shows ILR13RA2. FIG. 45C shows KLHL5. FIG. 45D shows PTX3. FIG. 45E shows GPX8. FIG. 45F shows IKBIP. FIG. 45G showsTXNDC15. FIG. 45H shows LY96. FIG. 45I shows RNF144B. FIG. 45J shows PDE4B. FIG. 45K shows C1S. FIG. 45L showsST8SIA4. FIG. 45M shows EDNRB. FIG. 45N shows ENTPD1. FIG. 45O shows WNT5A. FIG. 45P shows SAMSN1. FIG. 45Q shows MTMR11. FIG. 45R shows TLR1. FIG. 45S shows MME. FIG. 45T shows CACFD1. FIG. 45U shows CD69. FIG. 45V shows SNAPC1. FIG. 45W shows PRICKLE2. FIG. 45X shows SLAMF7. FIG. 45Y shows TSPAN2. FIG. 45Z shows CXCL6.

FIG. 46A, FIG. 46B, FIG. 46C, FIG. 46D, FIG. 46E, FIG. 46F, FIG. 46G, FIG. 46H, FIG. 46I, FIG. 46J, FIG. 46K, FIG. 46L, FIG. 46M, FIG. 46N, FIG. 46O, FIG. 46P, FIG. 46Q, FIG. 46R, FIG. 46S, FIG. 46T, FIG. 46U, FIG. 46V, FIG. 46W, FIG. 46X, FIG. 46Y and FIG. 46Z show the corresponding sensitivity and specificity at the cutoff point for a portion of the top 100 genes. FIG. 46A shows TNFRSF11B. FIG. 46B shows ACSL4. FIG. 46C shows CSGALNACT2. FIG. 46D shows DRAM1. FIG. 46E shows LILRB2. FIG. 46F shows PAPPA. FIG. 46G shows AKR1B1. FIG. 46H shows GPR183. FIG. 46I shows SGTB. FIG. 46J shows GLIPR1. FIG. 46K shows PDPN. FIG. 46L shows RBMS1. FIG. 46M shows SMARCA1. FIG. 46N shows ANGPT2. FIG. 46O shows PLAU. FIG. 46P shows TMEM55A. FIG. 46Q shows IGFBP5. FIG. 46R shows ASAP1. FIG. 46S shows SGCE. FIG. 46T shows HGF. FIG. 46U shows CEBPB. FIG. 46V shows DCBLD1. FIG. 46W shows MCTP1. FIG. 46X shows STAT4. FIG. 46Y shows ROBO1. FIG. 46Z shows ARL13B.

FIG. 47A, FIG. 47B, FIG. 47C, FIG. 47D, FIG. 47E, FIG. 47F, FIG. 47G, FIG. 47H, FIG. 47I, FIG. 47J, FIG. 47K, FIG. 47L, FIG. 47M, FIG. 47N, FIG. 47O, FIG. 47P, FIG. 47Q, FIG. 47R, FIG. 47S, FIG. 47T, FIG. 47U, FIG. 47V, FIG. 47W, FIG. 47X, FIG. 47Y and FIG. 47Z show the corresponding sensitivity and specificity at the cutoff point for a portion of the top 100 genes. FIG. 47A shows AAED1. FIG. 47B shows RGS5. FIG. 47C shows TOR1AIP1. FIG. 47D shows CCL18. FIG. 47E shows FERMT2. FIG. 47F shows BPGM. FIG. 47G shows NR3C1. FIG. 47H shows QKI. FIG. 47I shows STX11. FIG. 47J shows DEGS1. FIG. 47K shows THBD. FIG. 47L shows CCL2. FIG. 47M shows HS3ST3B1. FIG. 47N shows SDC2. FIG. 47O shows SLC16A10. FIG. 47P shows VCAN. FIG. 47Q shows PXDN. FIG. 47R shows SRGN. FIG. 47S shows DSE. FIG. 47T shows CAV1. FIG. 47U shows FGFR3. FIG. 47V shows ANGPTL2. FIG. 47W shows CLEC2B. FIG. 47X shows IL7R. FIG. 47Y shows CCR1. FIG. 47Z shows LAMC1.

FIG. 48A, FIG. 48B, FIG. 48C, FIG. 48D, FIG. 48E, FIG. 48F, FIG. 48G, FIG. 48H, FIG. 48I, FIG. 48J, FIG. 48K, FIG. 48L, FIG. 48M, FIG. 48N, FIG. 48O, FIG. 48P, FIG. 48Q, FIG. 48R, FIG. 48S, FIG. 48T, FIG. 48U and FIG. 48V show the corresponding sensitivity and specificity at the cutoff point for a portion of the top 100 genes. FIG. 48A shows LOX. FIG. 48B shows CFL2. FIG. 48C shows RDX. FIG. 48D shows SERPINE1. FIG. 48E shows CLIC2. FIG. 48F shows CLMP. FIG. 48G shows SNX10. FIG. 48H shows TNC. FIG. 48I shows FAM49A. FIG. 48J shows S100A9. FIG. 48K shows STC1. FIG. 48L shows ZNF57. FIG. 48M shows PPT1. FIG. 48N shows CYTIP. FIG. 48O shows CTSL. FIG. 48P shows GNB4. FIG. 48Q shows LDLRAD3. FIG. 48R shows RGS18. FIG. 48S shows THEMIS2. FIG. 48T shows BICC1. FIG. 48U shows HSPA13. FIG. 48V shows IL10RA.

FIG. 49 shows a CV plot.

FIG. 50 shows the sensitivity and specificity plot using 9 genes selected from the top 100 genes.

FIG. 51 shows a CV plot.

FIG. 52 shows the ROC curve based on the linear predictor constructed using the 5 genes only led to an AUC of 1 and improved sensitivity to 0.96.

FIG. 53 shows a prediction tree for IL13RA2.

### DETAILED DESCRIPTION

The present disclosure is based, at least in part, on the discovery that the plasminogen activation pathway plays a key role in driving colitis.

Because more and more expensive biologics are being approved for moderate to severe IBD and it is not currently known who should be treated with which one, the methods as described herein are extremely valuable. As anti-TNF therapy is still the first line, here we disclose a biomarker signature identifying subjects that won't respond to anti-TNF, which can be extremely attractive for the medical field and personalized medicine now that there are many alternative drugs.

A plasma or tissue biomarker of active disease is critically needed in IBD to aid physicians in assessing prognosis. Also a biomarker is needed to predict response to expensive biologic therapies and subset subjects for clinical trials to improve outcomes.

IL-17A is one of the most important and studied cytokines in intestinal inflammation (IBD or infection). But IL-17A may not be the culprit it has been made out to be. IL-17A appears to have both positive and negative effects based on mouse colitis models and human clinical trials with anti-IL-17A and anti-IL-17RA leading to more severe disease. Suggesting IL-17A is a poor drug target.

Despite the important role of IL-17 in autoimmunity and inflammatory bowel disease (IBD) its function in the mucosa in disease is still unclear. In IBD, IL-17 is produced in part by mucosal pro-inflammatory Th17 cells. However, multiple clinical trials using monoclonal therapy blocking IL-17 suggest that this cytokine actually plays a protective role in this disease. To investigate this possibility, we treated primary cultured intestinal epithelial cells with IL-17 and conducted transcriptomic analysis. Comparison of this IL-17-induced epithelial signature with transcriptomic analysis of biopsies from active versus inactive ulcerative colitis (UC) patients revealed a potential dysregulation of the coagulation pathway during active disease. We found that IL-17 induced epithelial cells to produce tissue plasminogen activator (tPA), and most UC patients had a marked upregulation of the direct tPA inhibitor, known as plasminogen activator inhibitor-1 (PAI-1). Based on these findings, we used both genetic and chemical inhibitor models to show that tPA was protective against damage by dextran sodium sulfate and Citrobacter infection, whereas PAI-1 exacerbated damage responses. We found that tPA inhibited inflammation in these models and that this was due to activation of the immunosuppressive molecule TGF-β. tPA cleaved the ubiquitous blood factor plasminogen, which in turn activated latent TGF-β to its mature form. This process was inhibited by PAI-1. Finally, we demonstrated that the level of colon PAI-1 in UC patients was predictive of both disease activity and response to biologic therapy. This study identifies a new pathway in UC where dysregulated PAI-1 leads to exacerbated inflammation and disease activity by blocking an IL-17-tPA-TGF-β axis.

Various aspects of these methods are described in more detail below.

### I. Methods

In an aspect, the disclosure provides a method of classifying a subject suffering from inflammatory bowel disease. The method generally comprises detecting the nucleic acid of one or more biomarkers selected from PAI-1/SERPINE, TNC, IL13RA2, CCL2, PRNP, GPX8, DRAM1, STAT4, IL24, IL6, PI15, PTGS2, SELE, SMR3A, SLC23A2, HDGFRP3, HIF1A, IKBIP, KLHL5, PTX3, TXNDC15, PDE4B, C1S, TLR1, MME, TSPAN2, TNFRSF11B, ACSL4, CSGALNACT2, SGTB, PDPN, RBMS1, ANGPT2, TMEM55A, HGF, RGS5, ROBO1, TOR1AIP1, CCL18, HS3ST3B1, SDC2, PXDN, DSE, SNX10, TNC, CLIC2, PPT1, RGS18, or THEMIS2 and classifying the subject as a responder or non-responder to treatment by determining the log 2 expression of the biomarker relative to a reference value. In reference to the above biomarkers, the sequence names can be identified in a public database, such as NCBI or UniProt and by using the gene name the markers are not limited to a specific species but when the biomarkers are used in the methods described herein the origin of the biomarker should match the species of the subject. In some embodiments, detecting a biomarker is selected from one or more of the group consisting of PAI-1/SERPINE, TNC, IL13RA2, CCL2, PRNP, GPX8, DRAM1, STAT4, IL24, IL6, PI15, PTGS2, SELE, SMR3A, SLC23A2, HDGFRP3, HIF1A, IKBIP, or KLHL5. In some embodiments, detecting a biomarker is selected from one or more of the group consisting of PRNP, IL13RA2, GPX8, IKBIP, KLHL5, PTX3, TXNDC15, PDE4B, C1S, TLR1, MME, TSPAN2, TNFRSF11B, ACSL4, CSGALNACT2, DRAM1, SGTB, PDPN, RBMS1, ANGPT2, TMEM55A, HGF, STAT4, RGS5, ROBO1, TOR1AIP1, CCL18, HS3ST3B1, SDC2, PXDN, DSE, SNX10, TNC, CLIC2, PPT1, RGS18, or THEMIS2.

Log 2 expression values for the genes studied herein can be from about 0 to about 20. For example, a log 2 expression value can be 0.1; 0.2; 0.3; 0.4; 0.5; 0.6; 0.7; 0.8; 0.9; 1; 1.1; 1.2; 1.3; 1.4; 1.5; 1.6; 1.7; 1.8; 1.9; 2; 2.1; 2.2; 2.3; 2.4; 2.5; 2.6; 2.7; 2.8; 2.9; 3; 3.1; 3.2; 3.3; 3.4; 3.5; 3.6; 3.7; 3.8; 3.9; 4; 4.1; 4.2; 4.3; 4.4; 4.5; 4.6; 4.7; 4.8; 4.9; 5; 5.1; 5.2; 5.3; 5.4; 5.5; 5.6; 5.7; 5.8; 5.9; 6; 6.1; 6.2; 6.3; 6.4; 6.5; 6.6; 6.7; 6.8; 6.9; 7; 7.1; 7.2; 7.3; 7.4; 7.5; 7.6; 7.7; 7.8; 7.9; 8; 8.1; 8.2; 8.3; 8.4; 8.5; 8.6; 8.7; 8.8; 8.9; 9; 9.1; 9.2; 9.3; 9.4; 9.5; 9.6; 9.7; 9.8; 9.9; 10; 10.1; 10.2; 10.3; 10.4; 10.5; 10.6; 10.7; 10.8; 10.9; 11; 11.1; 11.2; 11.3; 11.4; 11.5; 11.6; 11.7; 11.8; 11.9; 12; 12.1; 12.2; 12.3; 12.4; 12.5; 12.6; 12.7; 12.8; 12.9; 13; 13.1; 13.2; 13.3; 13.4; 13.5; 13.6; 13.7; 13.8; 13.9; 14; 14.1; 14.2; 14.3; 14.4; 14.5; 14.6; 14.7; 14.8; 14.9; 15; 15.1; 15.2; 15.3; 15.4; 15.5; 15.6; 15.7; 15.8; 15.9; 16; 16.1; 16.2; 16.3; 16.4; 16.5; 16.6; 16.7; 16.8; 16.9; 17; 17.1; 17.2; 17.3; 17.4; 17.5; 17.6; 17.7; 17.8; 17.9; 18; 18.1; 18.2; 18.3; 18.4; 18.5; 18.6; 18.7; 18.8; 18.9; 19; 19.1; 19.2; 19.3; 19.4; 19.5; 19.6; 19.7; 19.8; 19.9; or 20.

Plasminogen activator inhibitor-1 (PAI-1)(UniProt accession no. P05121) also known as endothelial plasminogen activator inhibitor or serpin E1 is a protein that in humans is encoded by the SERPINE1 gene. Elevated PAI-1 is a risk factor for thrombosis and atherosclerosis. PAI-1 is a serine protease inhibitor (serpin) that functions as the principal inhibitor of tissue plasminogen activator (tPA) and urokinase (uPA), the activators of plasminogen and hence fibrinolysis (the physiological breakdown of blood clots). It is a serine protease inhibitor (serpin) protein (SERPINE1). The PAI-1 gene is SERPINE1, located on chromosome 7 (7q21.3-q22).

In some embodiments, the subject is classified as a responder to anti-TNFα treatment if the log 2 expression value of PAI−PAI-1/SERPINE is less than about 6.5. In some embodiments, the subject is classified as a responder to anti-TNFα treatment if the log 2 expression value of TNC is less than about 6.3. In some embodiments, the subject is classified as a responder to anti-TNFα treatment if the log 2 expression value of IL13RA2 is less than about 5.5. In some embodiments, the subject is classified as a responder to anti-TNFα treatment if the log 2 expression value of CCL2 is less than about 7.5. In some embodiments, the subject is classified as a responder to anti-TNFα treatment if the log 2 expression value of PRNP is less than about 7.75. In some embodiments, the subject is classified as a responder to anti-TN Fa treatment if the log 2 expression value of GPX8 is less than about 5.5. In some embodiments, the subject is classified as a responder to anti-TNFα treatment if the log 2 expression value of DRAM1 is less than about 7.5. In some embodiments, the subject is classified as a responder to anti-TNFα treatment if the log 2 expression value of STAT4 is less than about 6.45. In some embodiments, the subject is classified as a responder to anti-TNFα treatment if the log 2 expression value of IKBIP is less than about 4.65. In some embodiments, the subject is classified as a responder to anti-TNFα treatment if the log 2 expression value of KLHL5 is less than about 5.25.

In some embodiments, the subject is classified as a non-responder to anti-TNFα treatment if the log 2 expression value of PAI-1/SERPINE is greater than about 6.5. In some embodiments, the subject is classified as a non-responder to anti-TNFα treatment if the log 2 expression value of TNC is greater than about 6.3. In some embodiments, the subject is classified as a non-responder to anti-TNFα treatment if the log 2 expression value of IL13RA2 is greater than about 5.5. In some embodiments, the subject is classified as a non-responder to anti-TNFα treatment if the log 2 expression value of CCL2 is greater than about 7.5. In some embodiments, the subject is classified as a non-responder to anti-TNFα treatment if the log 2 expression value of PRNP is greater than about 7.75. In some embodiments, the subject is classified as a non-responder to anti-TNFα treatment if the log 2 expression value of GPX8 is greater than about 5.5. In some embodiments, the subject is classified as a non-responder to anti-TNFα treatment if the log 2 expression value of DRAM1 is greater than about 7.5. In some embodiments, the subject is classified as a non-responder to anti-TNFα treatment if the log 2 expression value of STAT4 is greater than about 6.45. In some embodiments, the subject is classified as a non-responder to anti-TNFα treatment if the log 2 expression value of IKBIP is greater than about 4.65. In some embodiments, the subject is classified as a non-responder to anti-TNFα treatment if the log 2 expression value of KLHL5 is greater than about 5.25.

In another aspect, the disclosure provides a method of treating a subject suffering from inflammatory bowel disease. The method generally comprises (i) detecting the amount of one or more of PAI-1/SERPINE, TNC, IL13RA2, CCL2, PRNP, GPX8, DRAM1, STAT4, IKBIP, or KLHL5 in a biological sample obtained from the subject, (ii) determining the fold2 expression value relative to a reference value, (iii) classifying the subject as a responder or non-responder to anti-TNFα treatment and (iv) if the subject is classified as a responder, treating the subject with an anti-TNFα therapy or if the subject is classified as a non-responder, treating the subject with a PAI-1 inhibitor.

In yet another aspect, the disclosure provides a method of treating a subject in need thereof. The method generally comprises (i) detecting the amount of PAI-1/SERPINE in a biological sample obtained from the subject, (ii) diagnosing the subject with IBD when PAI-1 is upregulated relative to a reference value or if the PAI-1 log 2 expression value is greater than about 4.5, and (iii) administering an effective amount of an anti-TNF or anti-α4β7 antibodies to the subject if the PAI-1 levels have a log 2 expression value of 7.5 or less or administering an effective amount of a PAI-1 inhibitor if the PAI-1 levels have a log 2 expression value of about 9.5 or more. In some embodiments the anti-TNF antibody is infliximab. In some embodiments, the anti-α4β7 antibody is vedolizumab. In some embodiments, the PAI-1 inhibitor is CDE-268. In some embodiments, the subject has or is suspected of having IBD.

In still yet another aspect, the disclosure provides a method of treating a subject in need thereof. The method generally comprises (i) detecting the amount of PAI-1/SERPINE in a biological sample obtained from the subject, (ii) diagnosing the subject with active ulcerative colitis if the number of PAI-1 positive cells per high-power field is about 25 or greater, and (iii) administering an effective amount of an anti-TNF or anti-α4β7 antibodies to the subject if the PAI-1 levels have a log 2 expression value of 7.5 or less or administering an effective amount of a PAI-1 inhibitor if the PAI-1 levels have a log 2 expression value of about 9.5 or more. In some embodiments the anti-TNF antibody is infliximab. In some embodiments, the anti-α4β7 antibody is vedolizumab. In some embodiments, the PAI-1 inhibitor is CDE-268. In some embodiments, the subject has or is suspected of having IBD. As used herein, the term “high-power field” (HPF) is used in relation to microscopy, references the area visible under the maximum magnification power of the objective being used. In some embodiments, this represents a 400-fold magnification.

In another aspect, the disclosure provides a method of diagnosing or treating a subject in need thereof. The method generally comprises (i) obtaining a biological sample from a subject; (ii) detecting the level of PAI-1 and CCL2 in the sample; (iii) diagnosing the subject with IBD when PAI-1 is upregulated or the presence of PAI-1 is detected in the sample is greater than PAI-1 level in a control; diagnosing the subject with active ulcerative colitis if the number of PAI-1 positive cells per high-power field is about 25 or greater; or diagnosing the subject with IBD if PAI log 2 value is over 4.5; (iv) administering an effective amount of anti-TNF or anti-α4β7 antibodies (e.g., anti-TNFα (infliximab) and anti-α4β7 (vedolizumab)) to the diagnosed subject if the PAI-1 levels have a log 2 fold expression value of about 7.4 or less; (v) administering an effective amount of a PAI-1 inhibitor (e.g., CDE-268) if the PAI-1 levels have a log 2 fold expression value of about 9.2 or more; (vi) administering an effective amount of anti-TNF or anti-α4β7 antibodies (e.g., anti-TNFα (infliximab) and anti-α4β7 (vedolizumab)) to the diagnosed subject if the CCL2 levels have a log 2 fold expression value of about 9.2 or less; or (v) administering an effective amount of a PAI-1 inhibitor (e.g., CDE-268) if the CCL2 levels have a log 2 fold expression value of about 9.2 or more. In some embodiments, the subject has or is suspected of having IBD.

In yet another aspect, the disclosure provides a method of diagnosing or treating inflammatory bowel disease. The method generally comprises (i) obtaining a biological sample from a subject; (ii) detecting the level of PAI-1 in the sample; (iii) diagnosing the subject with IBD when PAI-1 is upregulated or the presence of PAI-1 is detected in the sample is greater than PAI-1 level in a control; diagnosing the subject with active ulcerative colitis if the number of PAI-1 positive cells per high-power field is about 25 or greater; or diagnosing the subject with IBD if PAI log 2 value is over 4.5; In some embodiments, the methods include (iv) administering an effective amount of anti-TNF or anti-α4β7 antibodies (e.g., anti-TNFα (infliximab) and anti-α4β7 (vedolizumab)) to the diagnosed subject if the PAI-1 levels have a log 2 fold expression value of about 7.5 or less; or (v) administering an effective amount of a PAI-1 inhibitor (e.g., CDE-268) if the PAI-1 levels have a log 2 fold expression value of about 9.5 or more.

In still yet another aspect, the disclosure provides a method of screening for a PAI-1 inhibitor capable of treating an inflammatory bowel disease. The method generally comprises (i) obtaining a biological sample from a subject; (ii) contacting the biological sample with a test compound; (iii) contacting a second biological sample with a lead compound; (ii) detecting the level of PAI-1 in the first biological sample or second biological sample; (iii) detecting interactions of chemicals or chemical moieties; or (iv) comparing the interactions of a test compound with a lead compound. In some embodiments, a test compound is identified as a PAI-1 inhibitor capable of treating an inflammatory bowel disease if the test compound decreases the level of PAI-1 or increases the level of tPA. In one aspect, this disclosure provides methods for identifying inhibitors of PAI-1 pathway. In one embodiment, the inhibitors of PAI-1 pathway are PAI-1 antagonists. The activity of a test agent may be evaluated based on the effect on any step of the PAI-1 pathway (as described in this disclosure). It can be compared to the effect in the absence of the test compound or may be compared to the effect of PAI-1 or a known antagonist thereof.

Assays to evaluate agents for inhibiting PAI-1 may be carried out by in vitro using purified or recombinant PAI-1. Assays can also be carried out in vitro using cells which express PAI-1—such as intestinal epithelial or non-epithelial cells. Further, screening test may be carried out in vivo using animal models. The cells in culture may be primary cells or may be secondary cells or cell lines. The cells may be enriched from sources such as the intestine. For example, tissue biopsy may be obtained from an individual and desired types of cells may be isolated using well known techniques or using commercially available kits. In one embodiment, the cells may be modified cells. For example, the cells may be engineered to express or overexpress PAI-1. The cells in culture can be maintained by using routine cell culture reagents and procedures. In one embodiment, the assays may be carried out in animals including mice.

The compounds for testing may be part of a library or may be newly synthesized. Further, the compounds may be purified, partially purified or may be present as cell extracts, crude mixtures and the like—i.e., unpurified. While it is ideal to test each compound separately, a combination of compounds may also be tested.

As used herein, the term “biological sample” refers to a sample obtained from a subject. Any biological sample containing IBD biomarkers is suitable. Numerous types of biological samples are known in the art. Suitable biological sample may include, but are not limited to, tissue samples or bodily fluids. In some embodiments, the biological sample is a tissue sample such as a tissue biopsy. The biopsied tissue may be fixed, embedded in paraffin or plastic, and sectioned, or the biopsied tissue may be frozen and cryosectioned. Alternatively, the biopsied tissue may be processed into individual cells or an explant, or processed into a homogenate, a cell extract, a membranous fraction, or a IBD biomarker extract. In other embodiments, the sample may be a bodily fluid. Non-limiting examples of suitable bodily fluids include blood, plasma, serum, urine, and saliva. In a specific embodiment, the biological sample is blood, plasma, or serum. In a specific embodiment, the biological sample is plasma. The fluid may be used “as is”, the cellular components may be isolated from the fluid, or a IBD biomarker fraction may be isolated from the fluid using standard techniques.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that the IBD biomarkers can be accurately detected and the amount measured according to the disclosure.

In some embodiments, a single sample is obtained from a subject to detect IBD biomarkers in the sample. Alternatively, IBD biomarkers may be detected in samples obtained over time from a subject. As such, more than one sample may be collected from a subject over time. For instance, 2, 3, 4, 5, 6, 7, 8, 9, 10, 11, 12, 13, 14, 15, 16 or more samples may be collected from a subject over time. In some embodiments, 2, 3, 4, 5, or 6 samples are collected from a subject over time. In other embodiments, 6, 7, 8, 9, or 10 samples are collected from a subject over time. In yet other embodiments, 10, 11, 12, 13, or 14 samples are collected from a subject over time. In other embodiments, 14, 15, 16 or more samples are collected from a subject over time.

When more than one sample is collected from a subject over time, samples may be collected every 0.5, 1, 2, 3, 4, 5, 6, 7, 8, 9, 10, 11, 12 or more hours. In some embodiments, samples are collected every 0.5, 1, 2, 3, or 4 hours. In other embodiments, samples are collected every 4, 5, 6, or 7 hours. In yet other embodiments, samples are collected every 7, 8, 9, or 10 hours. In other embodiments, samples are collected every 10, 11, 12 or more hours. Additionally, samples may be collected every 1, 2, 3, 4, 5, 6, 7, 8, 9, 10, 11, 12 or more days. In some embodiments, a sample is collected about every 6 days. In some embodiments, samples are collected every 1, 2, 3, 4, or 5 days. In other embodiments, samples are collected every 5, 6, 7, 8, or 9 days. In yet other embodiments, samples are collected every 9, 10, 11, 12 or more days.

In some embodiments, once a sample is obtained, it is processed in vitro to detect and measure the amount of IBD biomarkers. All suitable methods for detecting and measuring an amount of a IBD biomarker known to one of skill in the art are contemplated within the scope of the invention. In some embodiments, an IBD biomarker may be detected at the nucleic acid level. In some embodiments, an IBD biomarker may be detected at the protein level. For example, epitope binding agent assays (i.e. antibody assays), enzymatic assays, electrophoresis, chromatography and/or mass spectrometry may be used. Non-limiting examples of epitope binding agent assays include an ELISA, a lateral flow assay, a sandwich immunoassay, a radioimmunoassay, an immunoblot or Western blot, flow cytometry, immunohistochemistry, and an array. In one embodiment, IBD biomarkers are detected using PCR or qPCR. An IBD biomarker may be detected through direct infusion into the mass spectrometer. In another embodiment, IBD biomarkers are detected using chromatography. In particular, techniques linking a chromatographic step with a mass spectrometry step may be used. The chromatographic step may be liquid chromatography, gas chromatography or thin-layer chromatography (TLC). Generally speaking, the presence of IBD biomarkers may be determined utilizing liquid chromatography followed by mass spectrometry. In some embodiments, the liquid chromatography is high performance liquid chromatography (HPLC). Non-limiting examples of HPLC include partition chromatography, normal phase chromatography, displacement chromatography, reverse phase chromatography, size exclusion chromatography, ion exchange chromatography, bioaffinity chromatography, aqueous normal phase chromatography or ultrafast liquid chromatography. Non-limiting examples of mass spectrometry include constant neutral loss mass spectrometry, tandem mass spectrometry (MS/MS), matrix-assisted laser desorption/ionization (MALDI), electrospray ionization mass spectrometry (ESI-MS).

Any suitable reference value known in the art may be used. For example, a suitable reference value may be the amount of a IBD biomarker in a biological sample obtained from a subject or group of subjects of the same species that has no detectable IBD. In another example, a suitable reference value may be the amount of a IBD biomarker in a biological sample obtained from a subject or group of subjects of the same species that has detectable IBD as measured via standard methods. In another example, a suitable reference value may be a measurement of the amount of an IBD biomarker in a reference sample obtained from the same subject. The reference sample comprises the same type of biological fluid as the test sample, and may or may not be obtained from the subject when IBD was not suspected. A skilled artisan will appreciate that it is not always possible or desirable to obtain a reference sample from a subject when the subject is otherwise healthy. For example, in an acute setting, a reference sample may be the first sample obtained from the subject at presentation. In another example, when monitoring the effectiveness of a therapy, a reference sample may be a sample obtained from a subject before therapy began. In such an example, a subject may have suspected IBD but may not have other symptoms of IBD or the subject may have suspected IBD and one or more other symptom of IBD. In a specific embodiment, a suitable reference value may be a threshold provided in the Examples.

In another aspect, the disclosure provides a method of treating IBD in a subject in need thereof. The method generally comprises (i) administering a therapeutically effect amount of tissue plasminogen activator (tPA). Tissue plasminogen activator (UniProt Accession No. P00750)(abbreviated tPA or PLAT) is a protein involved in the breakdown of blood clots. It is a serine protease (EC 3.4.21.68) found on endothelial cells, the cells that line the blood vessels. As an enzyme, it catalyzes the conversion of plasminogen to plasmin, the major enzyme responsible for clot breakdown. Human tPA has a molecular weight of ˜70 kDa in the single-chain form.

tPA can be manufactured using recombinant biotechnology techniques; tPA produced by such means are referred to as recombinant tissue plasminogen activator (rtPA). Specific rtPAs include alteplase, reteplase, and tenecteplase. They are used in clinical medicine to treat embolic or thrombotic stroke. The use of this protein is contraindicated in hemorrhagic stroke and head trauma. The antidote for tPA in case of toxicity is aminocaproic acid. tPA is used in some cases of diseases that feature blood clots, such as pulmonary embolism, myocardial infarction, and stroke, in a medical treatment called thrombolysis. The most common use is for ischemic stroke.

Methods described herein are generally performed on a subject in need thereof. A subject in need of the therapeutic methods described herein can be a subject having, diagnosed with, suspected of having, or at risk for IBD. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a therapeutic agent is, for example, that amount that would cause the desired therapeutic effect in a subject while minimizing undesired side effects. In various embodiments, an effective amount of a therapeutic agent described herein can substantially inhibit or mitigate IBD and/or related symptoms.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit or mitigate IBD or related symptoms.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a therapeutic agent can occur as a single event or over a time course of treatment. For example, a therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a cardiovascular disease, disorder, or condition.

A therapeutic agent can be administered simultaneously or sequentially with another agent, such as standard therapeutic for IBD or another agent. For example, a therapeutic agent can be administered simultaneously with another agent, such as a standard IBD therapeutic. Simultaneous administration can occur through administration of separate compositions, each containing one or more of a therapeutic agent or another agent. Simultaneous administration can occur through administration of one composition containing two or more of a therapeutic agent or another agent. A therapeutic agent can be administered sequentially with an antibiotic, an anti-inflammatory, or another agent. For example, a therapeutic agent can be administered before or after administration of an antibiotic, an anti-inflammatory, or another agent.

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 5th Ed., Smith, M. B. and March, J., eds. John Wiley & Sons, New York: 2001, the entire contents of which are hereby incorporated by reference.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction

### Example 1: IL-17A Added to the Intestinal Epithelium

What happens when you add IL-17A to the intestinal epithelium? We have more than half dozen functional assays with IL-17A (not shown), but shown here is the microarray analysis.

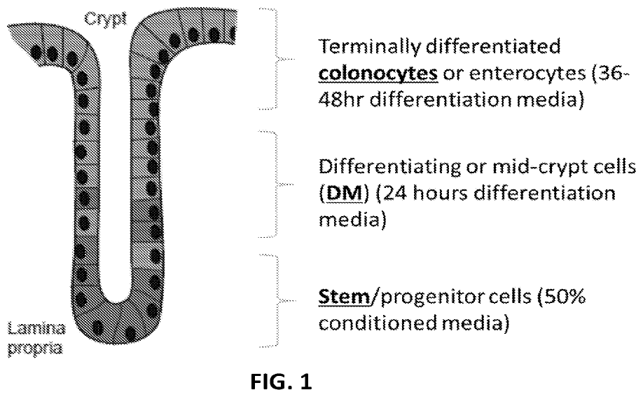

FIG. 1 shows an in vitro culture system (Kaiko G and Ryu S et al, Cell, 2016).

Microarray Experiment Setup: Primary Epithelial Cells

From colon epithelial lines n=4; Stem cells; Stem cells (2 days); DM; Colonocytes; With and without 20 ng/ml IL-17A.

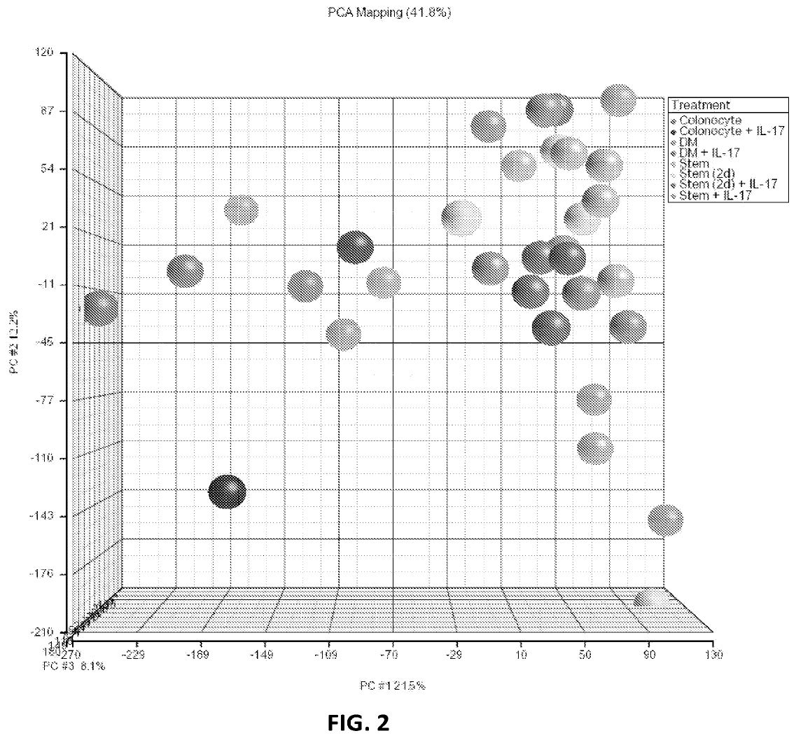

FIG. 2. PCA plot: IL-17 had subtle effect relative to stem cell differentiation.

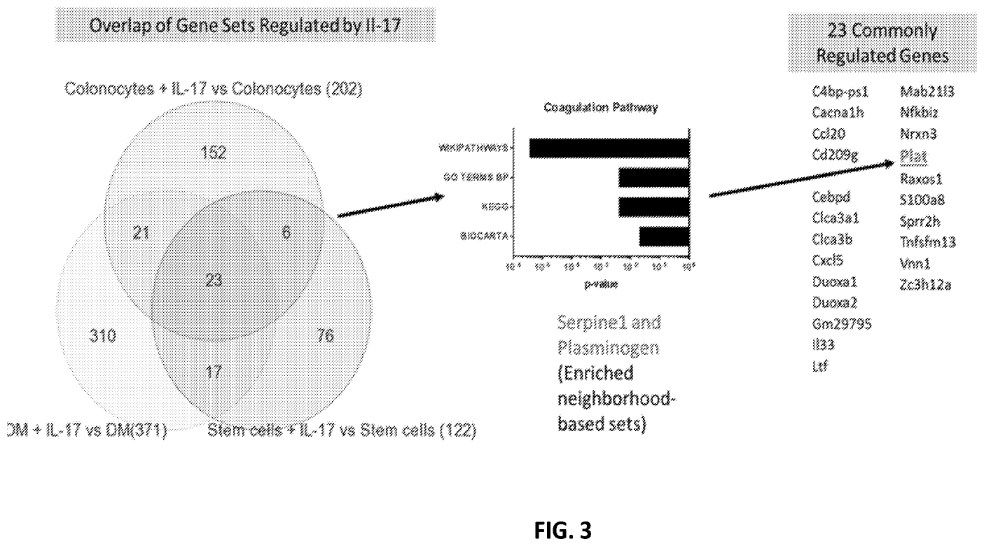

FIG. 3 Identification of gene candidates downstream of IL-17A in the epithelium

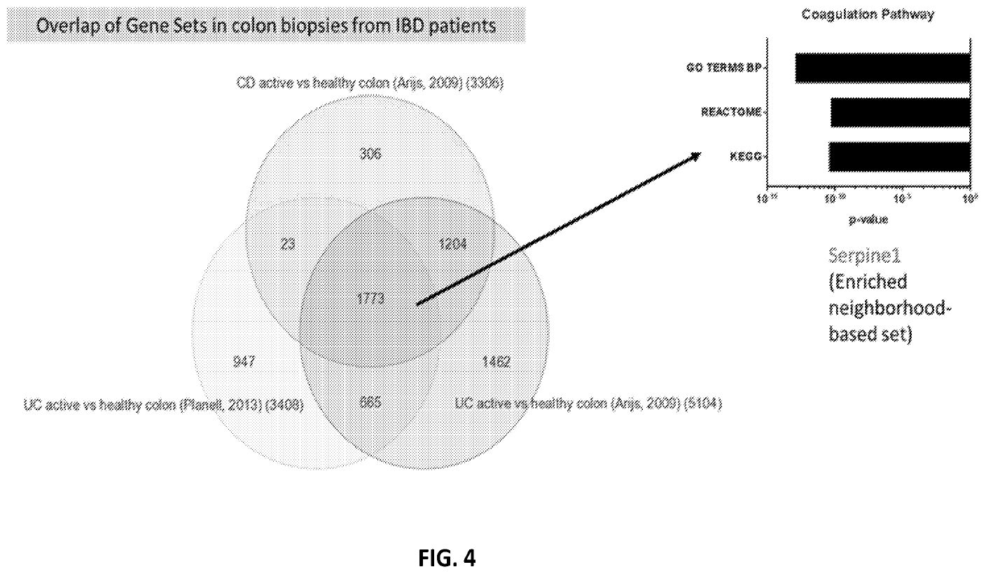

FIG. 4 Identification of genes with conserved dysregulation amongst IBD patients.

Plat was the most up- or down-regulated gene in the coagulation pathway altered by IL-17. Why is a member of the coagulation cascade altered by immune activation through IL-17 on colon epithelial cells? Does this pathway give a previously overlooked insight into disease pathogenesis?

Array Analysis: ImmGen Database

Cross-referencing with ImmGen identified that Plat was a gene upregulated by endothelial, and fibroblast cells upon immune stimulation. Suggests it has some inflammatory role as yet unknown. Most other commonly regulated genes were innate immune molecules.

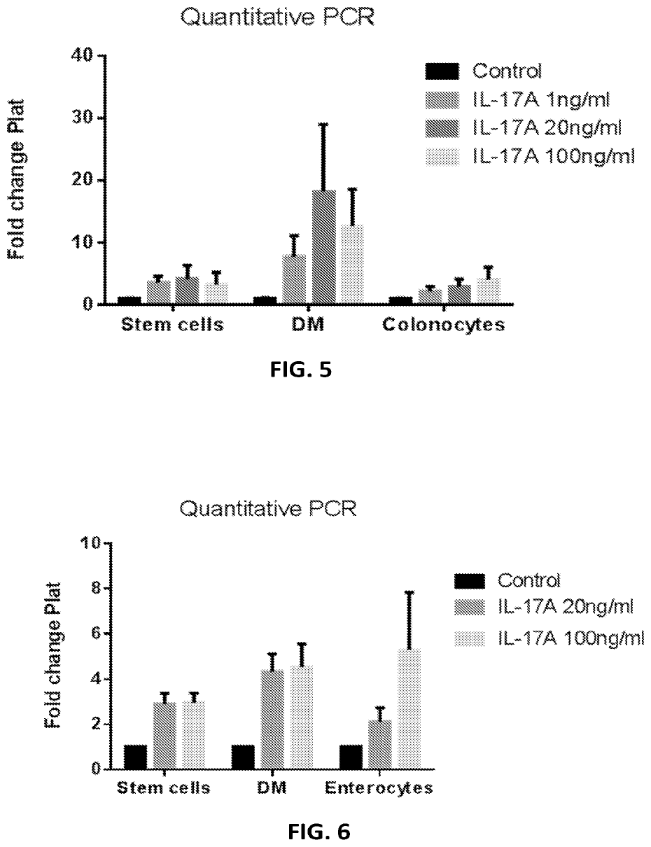

FIG. 5 qPCR verification and dose curve: colon.

FIG. 6 Dose curve: Ileum.

IL-17A has a conserved association with tPA. GEO dataset mining shows tPA strongly linked to both colitis disease state and IL-17A levels in humans and mice. By searching GEO data array sets for skin epidermis/keratinocytes and lung epithelium treated with IL-17A it was clear that Plat up-regulation was a conserved epithelial response to IL-17A. Plat mRNA is upregulated in IL-17-dominated intestinal models, such as DSS and Citrobacter rodentium infection (˜4-fold). Serpine1 mRNA upregulated ˜7-fold in DSS but not in Citrobacter. However, no one has examined why IL-17A is linked to Plat in any organ system. So what is Plat or tissue plasminogen activator (tPA)?

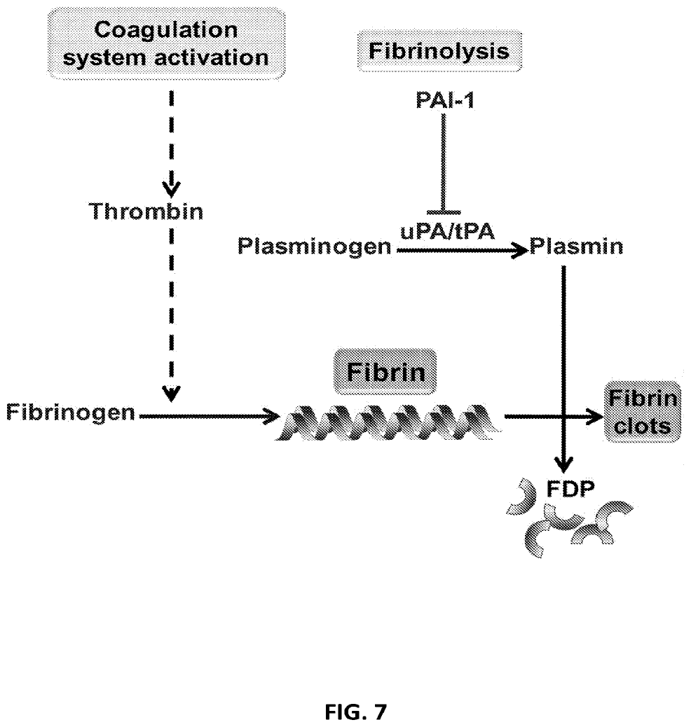

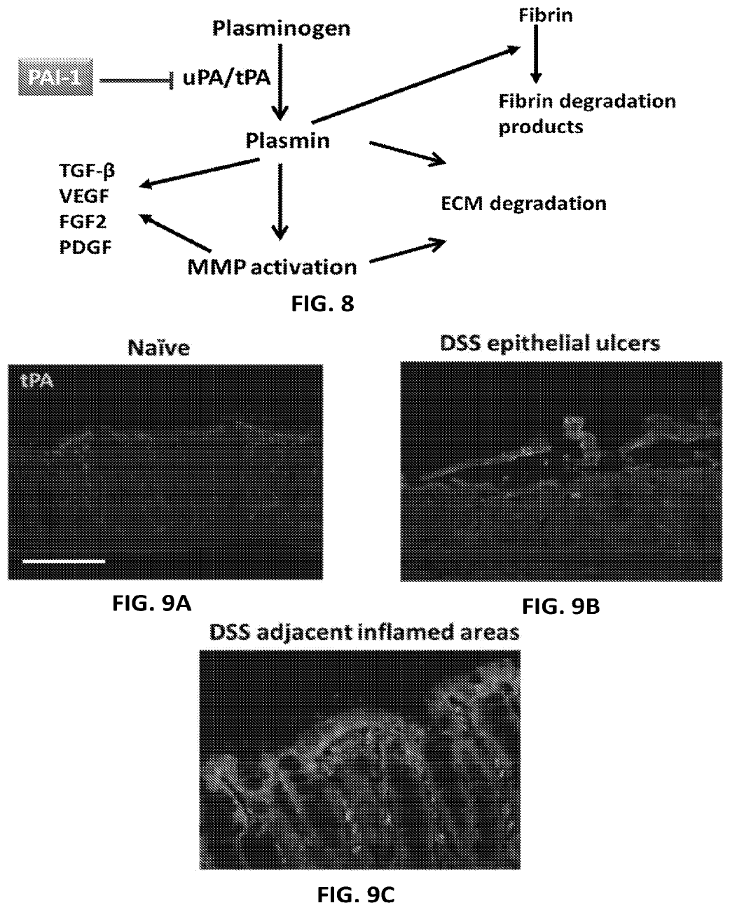

FIG. 7 Textbook view of tPA and its inhibitor PAI-1. tPA and PAI-1 are far more than just clotting factors. tPA is a serine protease with plasm in dependent and independent functions. Inhibition of PAI-1 potentiates these novel functions of tPA (see e.g., FIG. 8). tPA and PAI-1 (pathway not functionally studied in IBD). PAI-1 is a direct binding inhibitor of tPA. IBD patients are at much greater risk (3×) of thrombosis and hyper-coagulation disorders (˜90% of IBD patients have abnormal hemocoagulation parameters—Kohoutova D et al., Scand J Gastro, 2014). tPA/PAI-1 heavily studied in neuronal and cardiovascular system with a potential role in remodeling/cell migration

Hypothesis

tPA is an anti-inflammatory, pro-repair molecule that acts as a positive downstream effector of IL-17A. Increasing the levels of tPA (e.g. by inhibiting PAI-1) may have potential as a novel drug therapy in IBD not only improving disease outcome but also reducing thrombotic risk. tPA is expressed in vivo in response to IL-17-inducing colitis models.

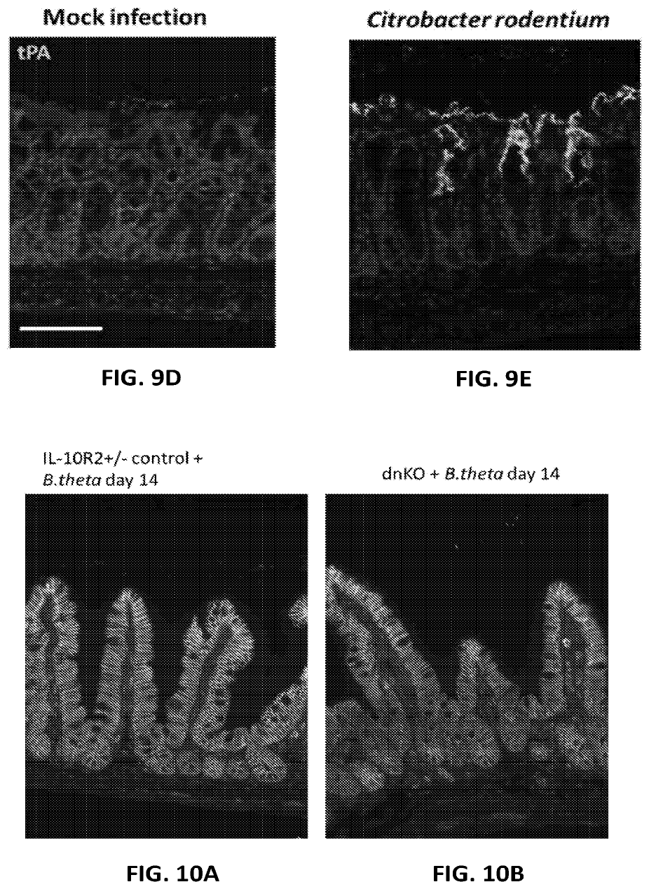

FIG. 9 tPA is induced by inflammation and derived from epithelial and non-epithelial cells in the mouse.

FIG. 10 tPA is induced by inflammation and derived from epithelial and non-epithelial cells in the mouse. No tPA in any CRF het at day 14 and none in any mice at day 0 in the ileum.

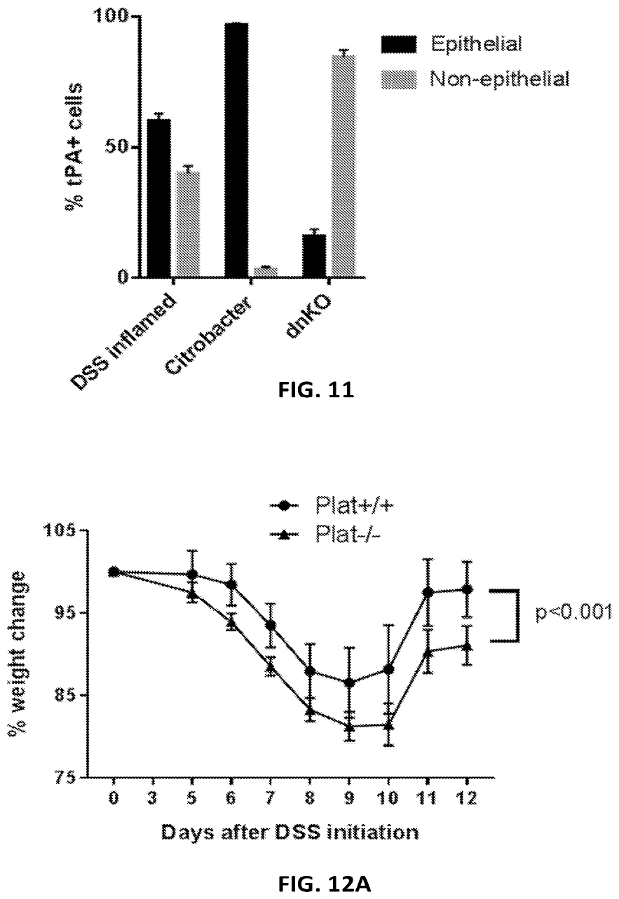

FIG. 11. tPA is low to absent without inflammation and derived from epithelial and non-epithelial cells in the mouse.

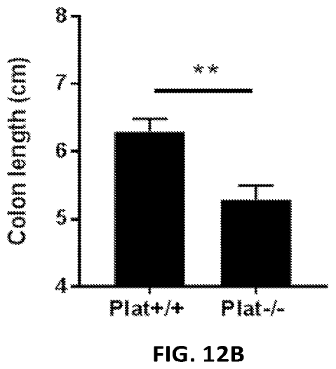

FIG. 12A-FIG. 12B. Data suggests tPA protects against colitis.

### Example 2: A Novel PAI-1 Inhibitor

PAI-1 inhibitor (CDE-268) developed from a small molecule screen.

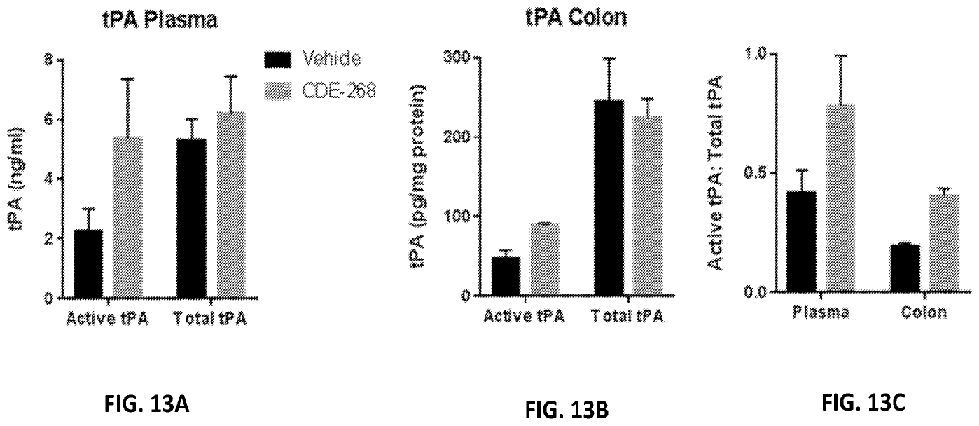

FIG. 13. A novel PAI-1 inhibitor elevates tPA levels in the blood and colon.

Studying the function of tPA in disease, and PAI-1 inhibitor as a novel therapy using DSS colitis.

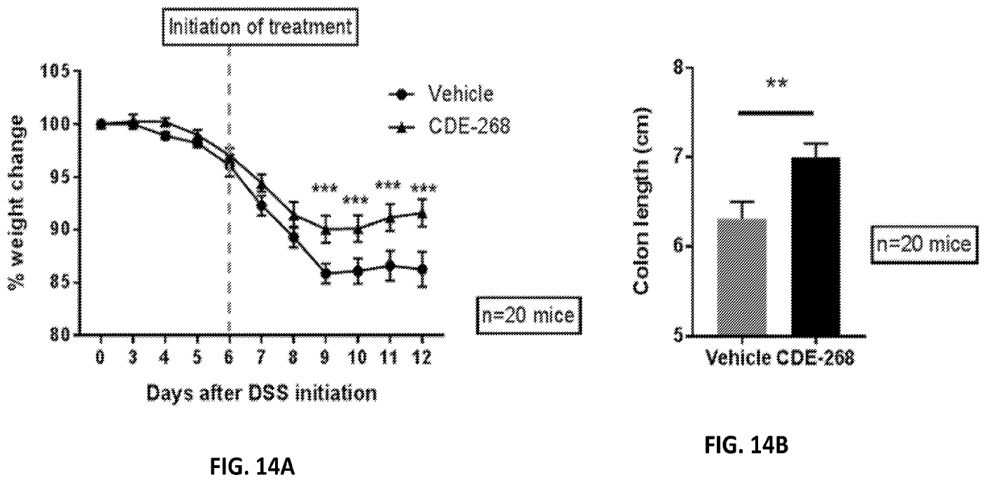

FIG. 14 shows targeting PAI-1 as a therapy (not prophylaxis) in DSS colitis suppresses disease.

Better than therapeutic results achieved with prednisone or anti-IL-6 in mice and comparable to therapeutic results of anti-TNFα in mice.

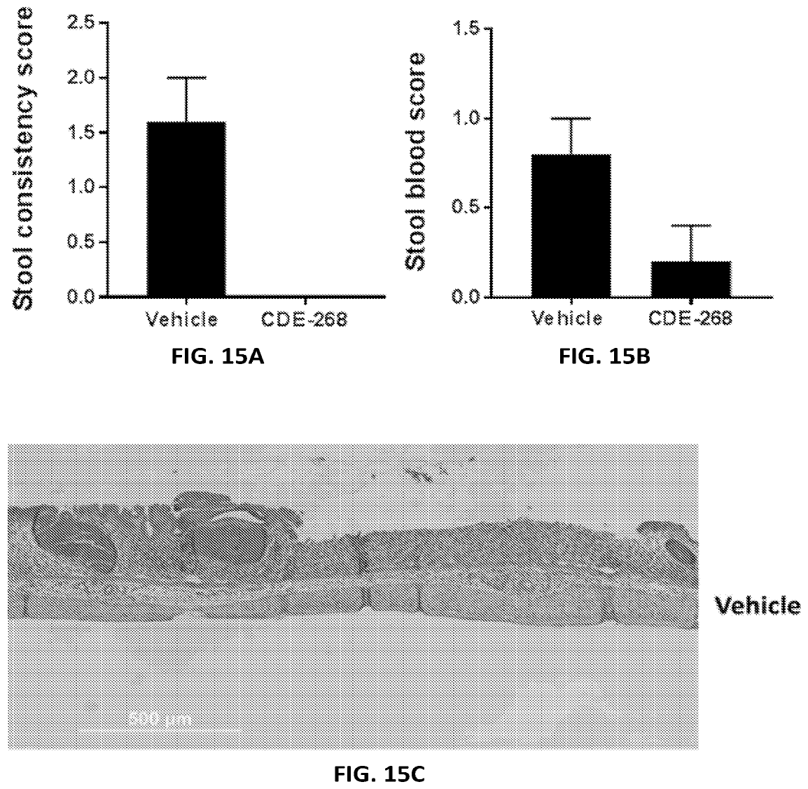

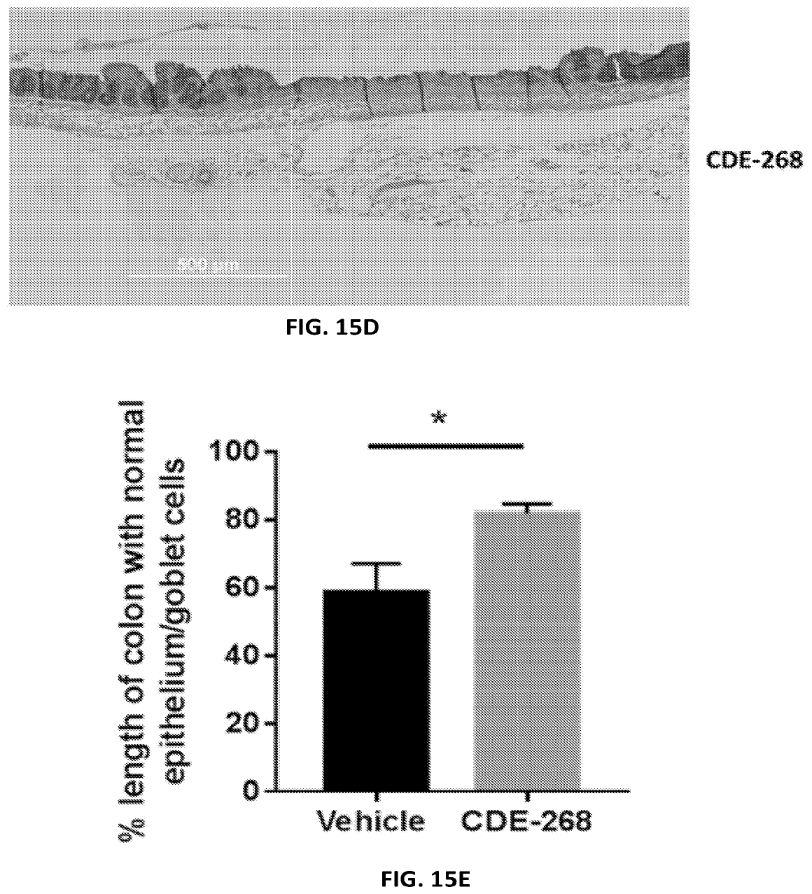

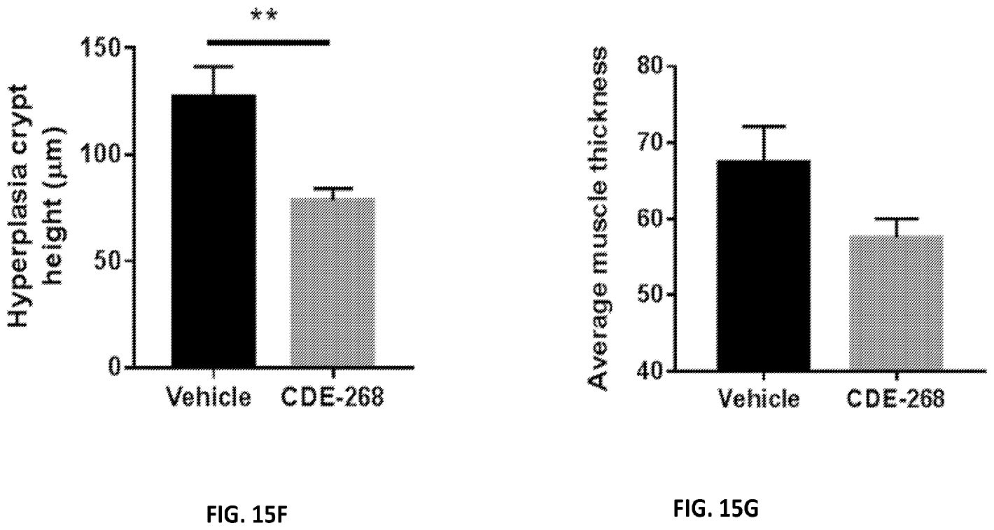

FIG. 15 shows targeting PAI-1 as a therapy (not prophylaxis) in DSS colitis suppresses disease.

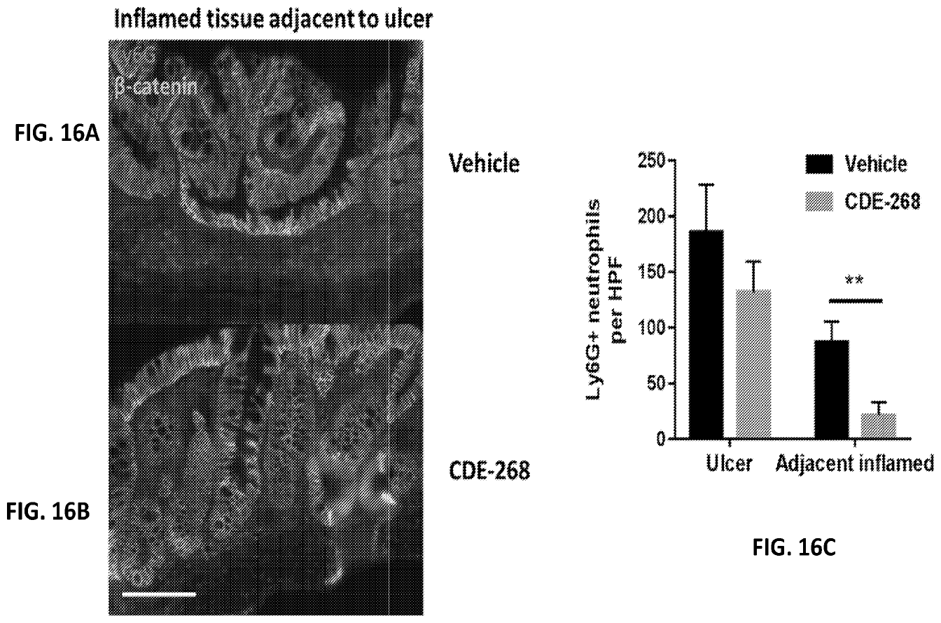

FIG. 16 PAI-1 inhibition suppresses neutrophil influx.

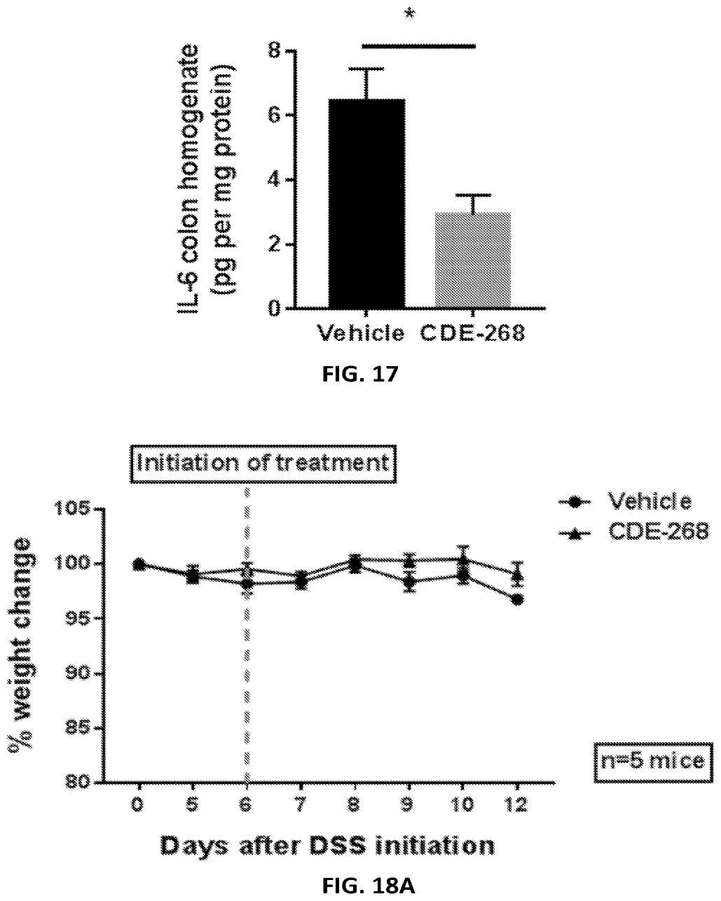

FIG. 17 PAI-1 inhibition suppresses IL-6.

Studying the function of tPA in disease, and PAI-1 inhibitor as a novel therapy using Citrobacter rodentium colitis.

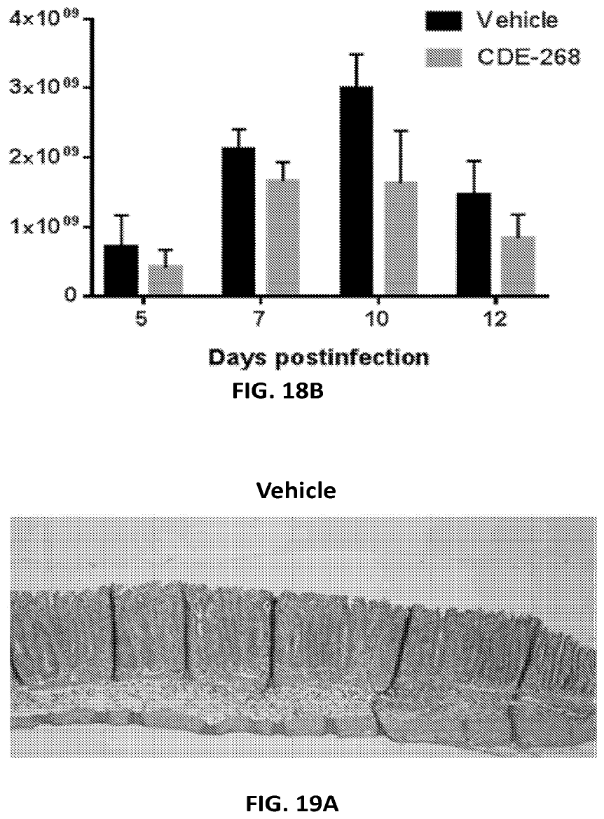

FIG. 18 Trend to reduced weight loss and bacterial burden with PAI-1 inhibition. Importantly though inhibitor does not worsen bacterial infection, which was one of the deleterious effects and major concerns of anti-IL-17 treatment in clinical trials and also mouse models.

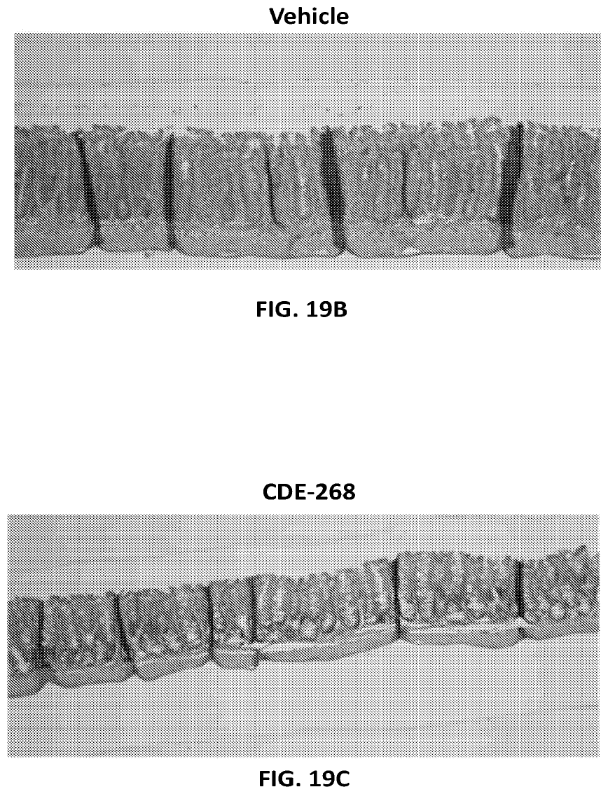

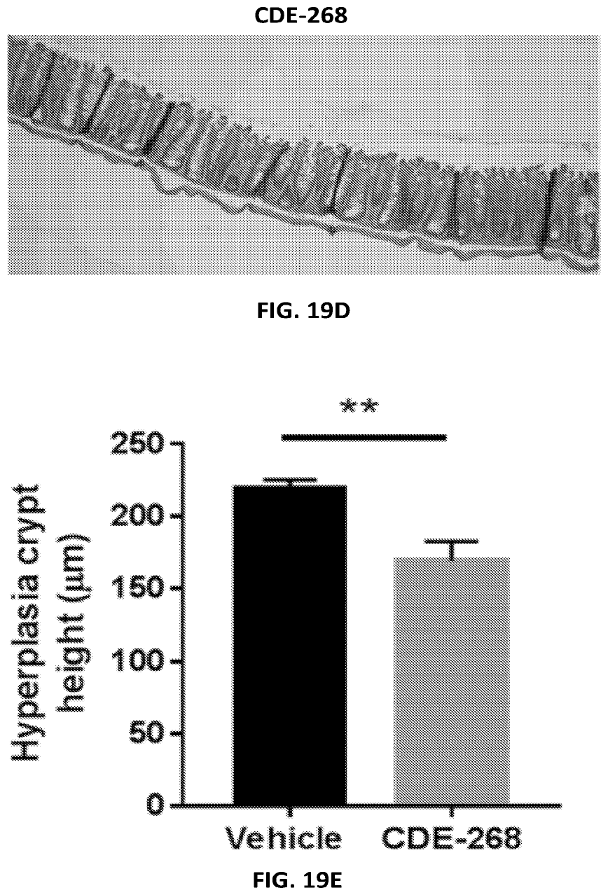

FIG. 19. PAI-1 inhibition suppresses crypt hyperplasia.

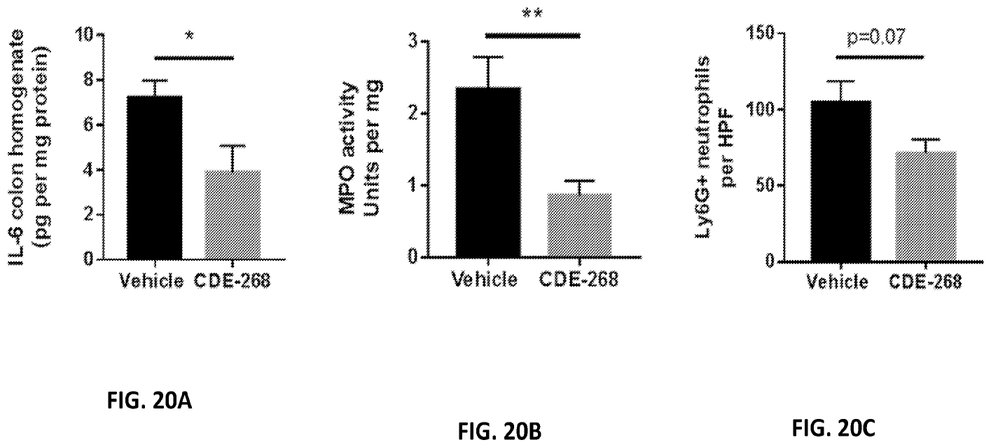

FIG. 20. PAI-1 inhibition suppresses IL-6 and MPO activity

Mechanism of Action

What upstream signaling pathway drives Plat/tPA through IL-17A?

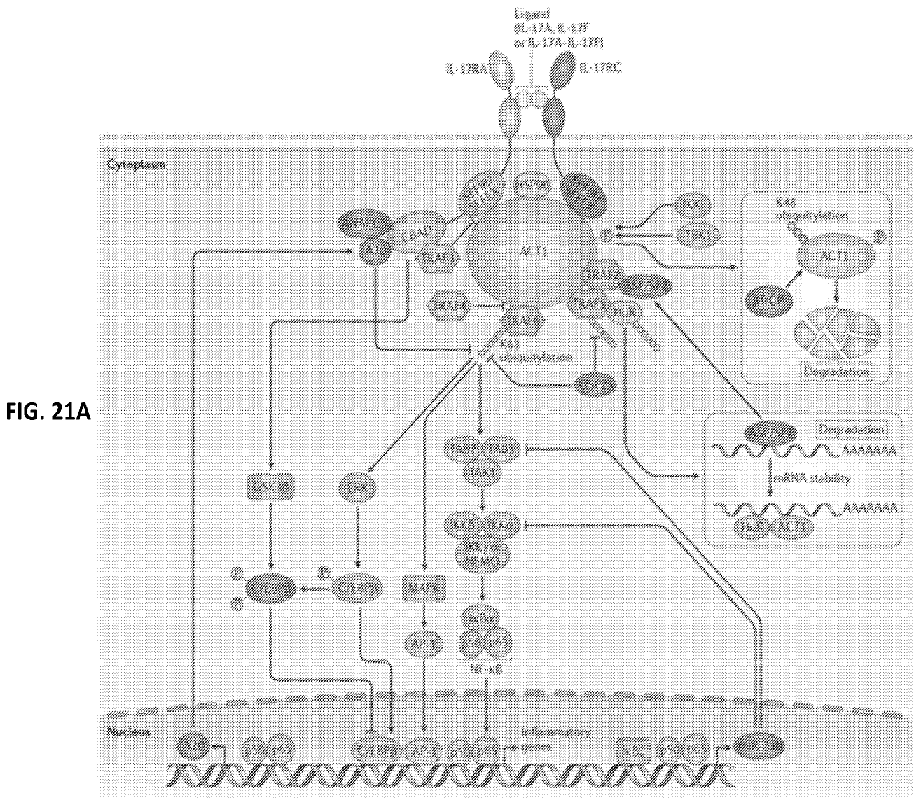

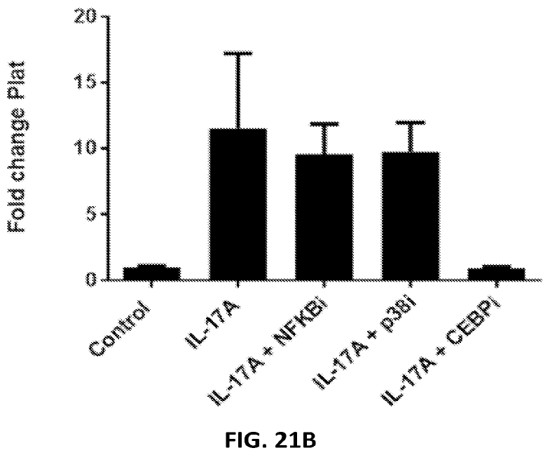

FIG. 21 IL-17RA signaling. Cebpd is also one of the Venn diagram genes upregulated by IL-17.

Downstream Signaling Pathway from tPA

TGF-β is a immunosuppressive/repair modulatory molecule that sits in the ECM and needs to be cleaved by a protease to be activated.

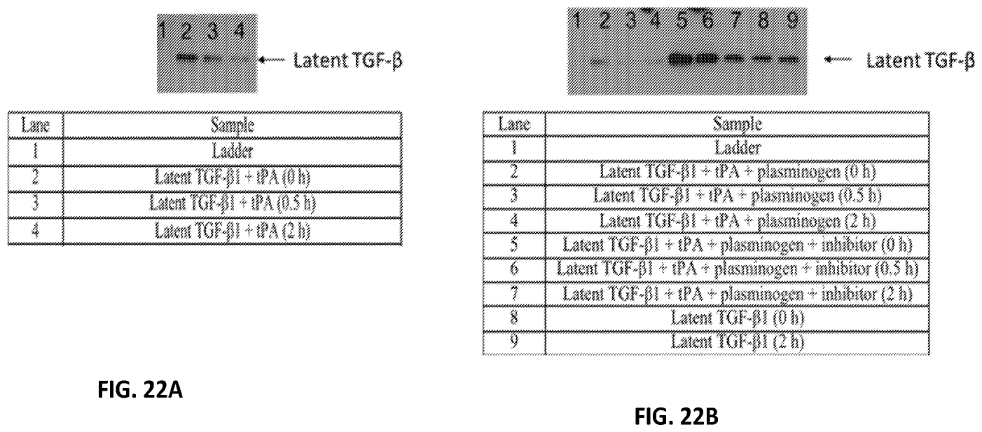

FIG. 22. Evidence suggests tPA can directly and indirectly cleave latent TGFβ in cell free assay.

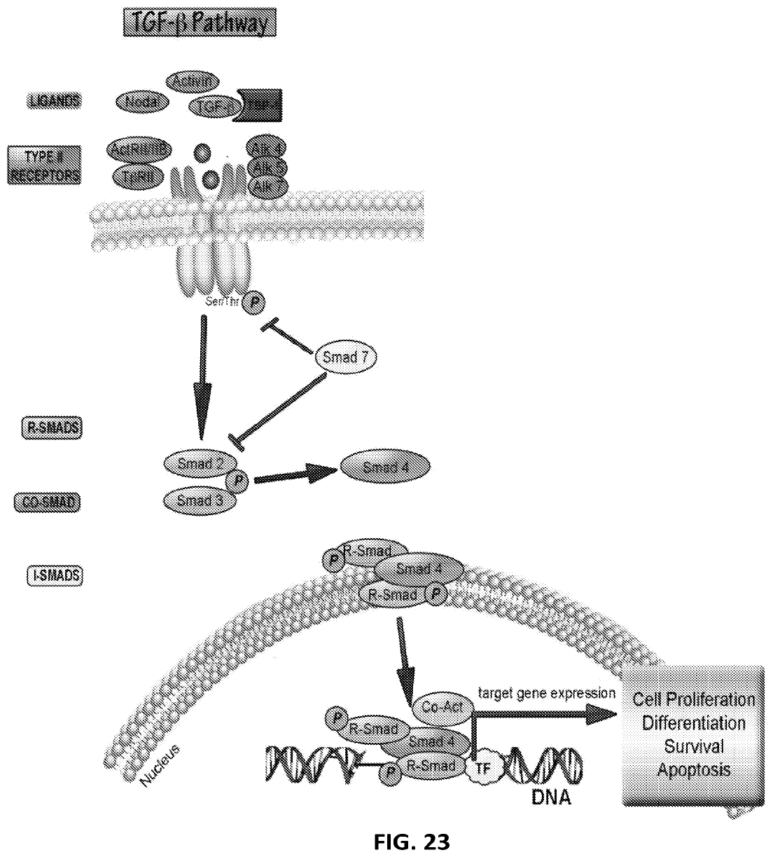

FIG. 23. TGF-β pathway. In cancer cell lines most highly upregulated gene is serpin1/PAI-1.

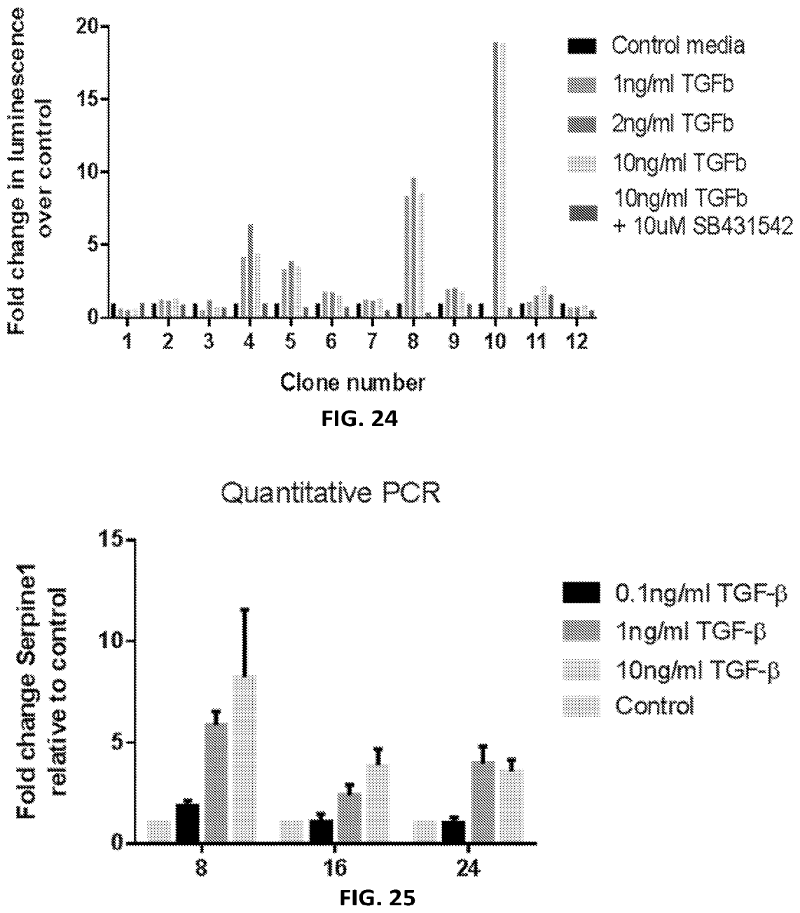

FIG. 24. Construction of a TGFβ-Smad-luciferase reporter. Isolated 12 clones and tested responsiveness to TGFβ (mouse and human) Clone #8 and 10 chosen and expanded to make a stable line for testing supernatants and colon homogenates for mature TGFβ activity. TGF-beta reporter activity assay results confirm western blots. I-ling for data

FIG. 25. TGFβ drives serpine1/PAI-1 expression in the colon spheroids (negative feedback loop)

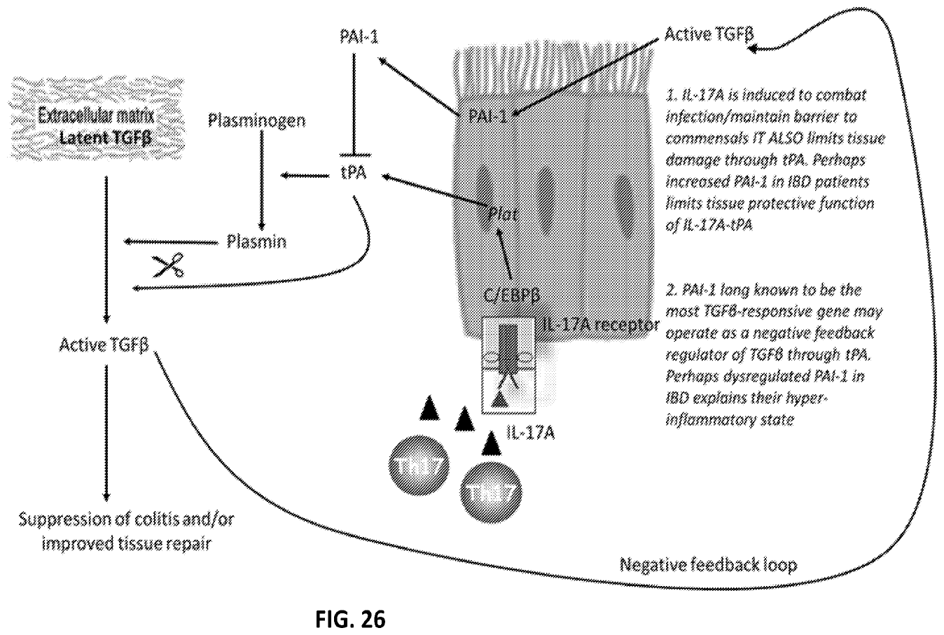

FIG. 26. 1. IL-17A is induced to combat infection/maintain barrier to commensals IT ALSO limits tissue damage through tPA. Perhaps increased PAI-1 in IBD patients limits tissue protective function of IL-17A-tPA. 2. PAI-1 long known to be the most TGFβ-responsive gene may operate as a negative feedback regulator of TGF through tPA. Perhaps dysregulated PAI-1 in IBD explains their hyper-inflammatory state. Models of interest include dnKO colitis model, PlatKO mice, DSS colitis model (remainder of endpoints). Test whether immune cells replicate effect of recombinant IL-17A-tPA induction in vitro (Th17 cells+colonic epithelial cells) (Australia). Test whether genetic increase in tPA improves colitis outcomes using PAI-1KO mice in DSS. Downstream mechanism: Test whether tPA acts as a protease cleaves and activates a latent form TGFb. Replace TGFb in PlatKO mice undergoing DSS OR inhibit it in PAI-1 inhibitor treated mice undergoing DSS, to show functional role downstream of tPA.

### Example 3: PAI-1 is Elevated in IBD Patients

What about humans and what may be going wrong with this pathway in IBD? It was hypothesized that due to inflammation and tissue damage patients with active IBD have elevated PAI-1 which disrupts the tPA/TGF-β axis. IT was shown that PAI-1 is elevated in IBD patients, herein. Human IBD patients great need for: 1. Biomarker for disease activity? 2. Predictor of biologic therapy response?

tPA and PAI-1 (Pathway not Functionally Studied in IBD).

PAI-1 is a direct binding inhibitor of tPA. IBD patients are at much greater risk (3×) of thrombosis and hyper-coagulation disorders (˜90% of IBD patients have abnormal hemocoagulation parameters—Kohoutova D et al., Scand J Gastro, 2014).

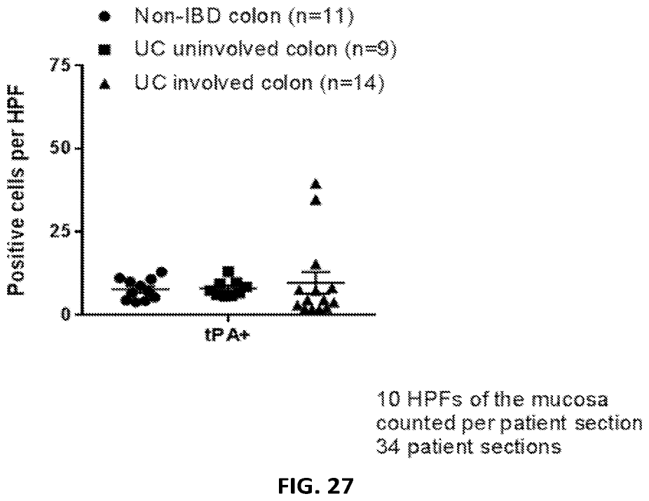

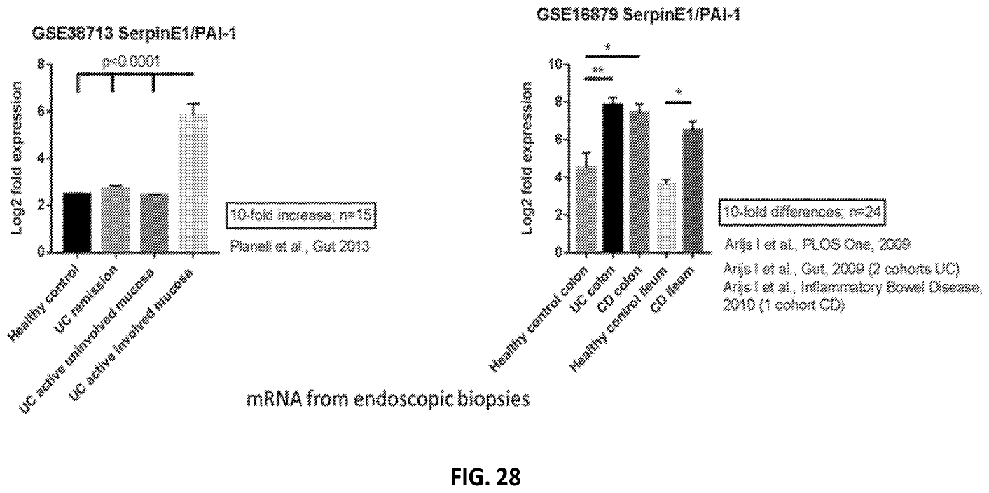

FIG. 27. tPA is not altered in UC patients, IF staining of sections from surgical resection cases. Therefore, tPA is not a biomarker.

FIG. 28. Serpine1/PAI-1 highly up-regulated in inflamed tissue from CD and UC patients (4 cohorts) analysis of deposited raw data in GEO NCBI.

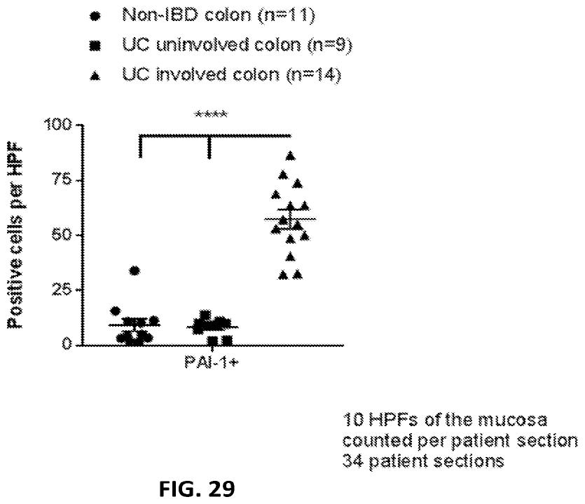

FIG. 29. PAI-1 protein highly up-regulated in inflamed tissue from UC patients, IF staining of sections from surgical resection cases.

Therefore, Serpine1/PAI-1 expression in colon tissue indicates disease activity in UC (diagnostic/prognostic potential).

### Example 4: Predictors of which Patients Will Respond to Biologic Therapy Anti-TNFα (Infliximab) and Anti-α4β7 (Vedolizumab)

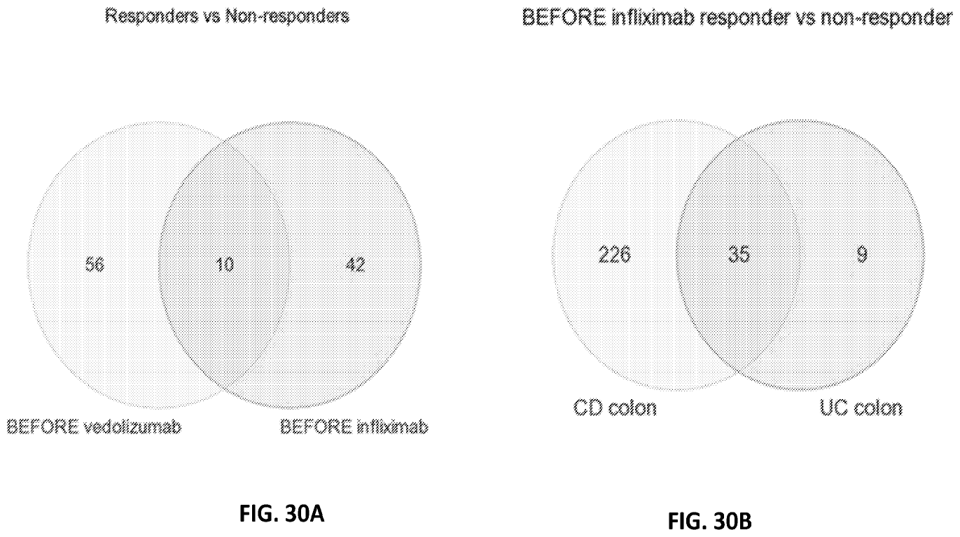

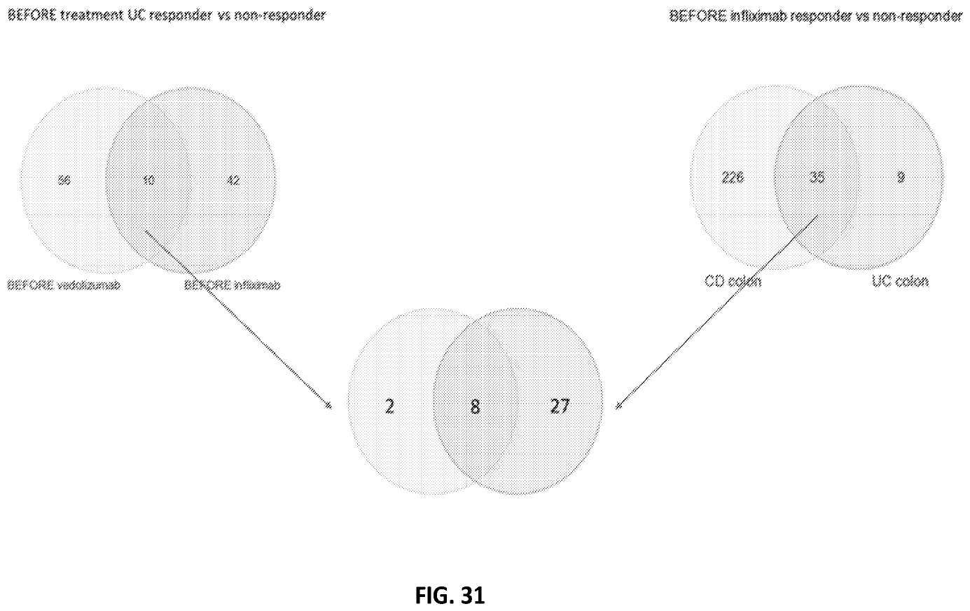

Process of identifying UC predictive signature to indicate response to biologics. Colon biopsy mRNA microarray raw data deposited in GEO NCBI. Here, we target mined the raw data which had to be deposited into GEO NCBI. ˜300 patients across 3 separate cohorts. See e.g., FIG. 30 and FIG. 31.

Biopsies were taken from moderate-to-severe IBD patients in multiple cohorts before beginning therapy with a monoclonal biologic drug. Microarrays were conducted on these biopsies. We made various comparisons across studies of the genes that were altered BEFORE treatment in patients that would later go on to either RESPOND versus NOT RESPOND to therapy. Therefore, these genes are predictive of how likely the patient is to RESPOND to the drug.

We then compiled these comparisons and made an 8 gene colon signature to predict a patient's response to the drug (see e.g., FIG. 31).

TABLE 1
8 gene biomarker signature.
8 Gene Biomarker Signature

SERPINE1/PAI-1
CCL2
IL24
IL6
PI15
PTGS2
SELE
TNC

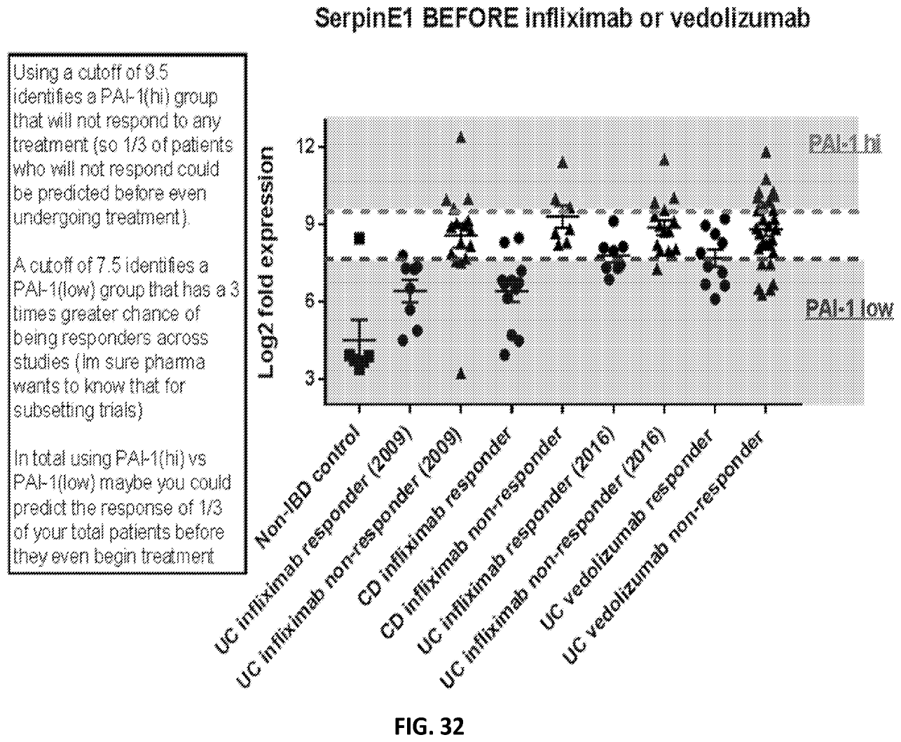

High Serpine1 expressing patients are less likely to respond to either infliximab or vedolizumab (see e.g., FIG. 32). The accuracy of prediction improved when using all genes from 8 gene signature. It is believed that this study is possibly largest ever UC transcriptional analysis performed by us (10 independent studies across multiple continents on different array platforms over 8 years). It was found that PAI-1 consistently upregulated in active UC biopsies in all studies and PAI-1 strongly correlates with inflammatory molecules in UC patient routine biopsies.

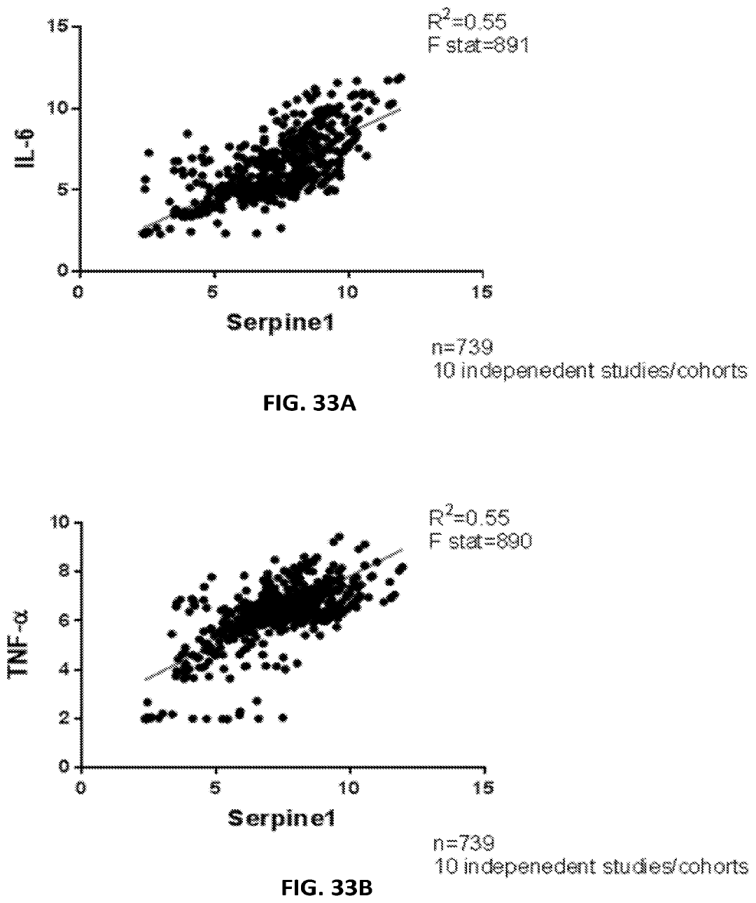

FIG. 33 shows a positive correlation between PAI-1 and IL-6/TNF-α.

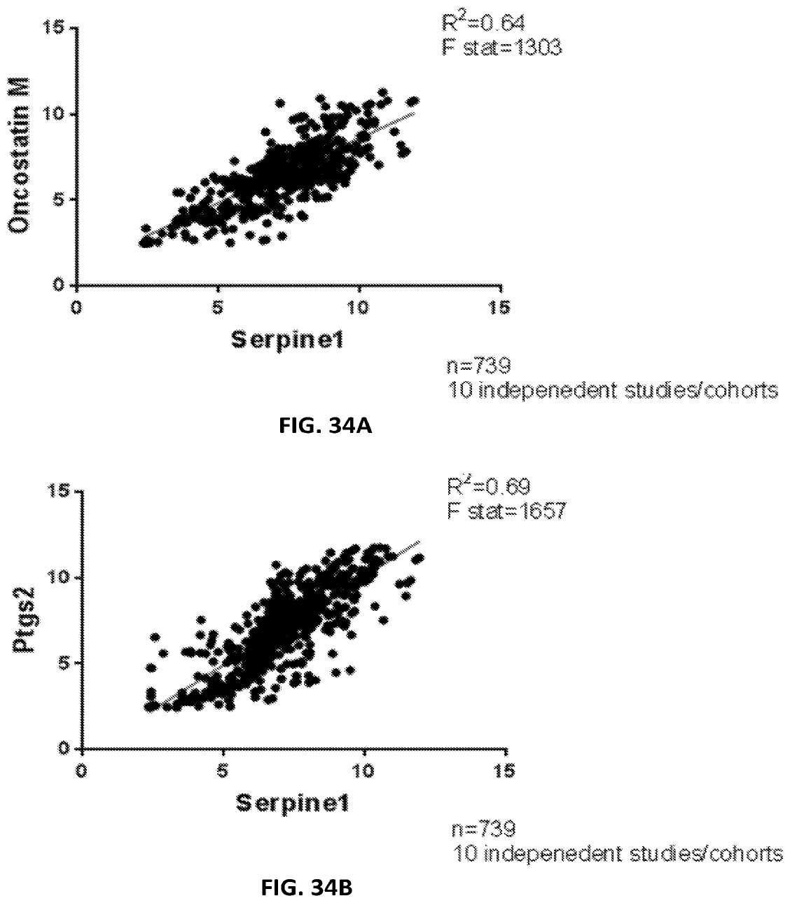

FIG. 34 shows a positive correlation between PAI-1 and OncostatinM/Cox2.

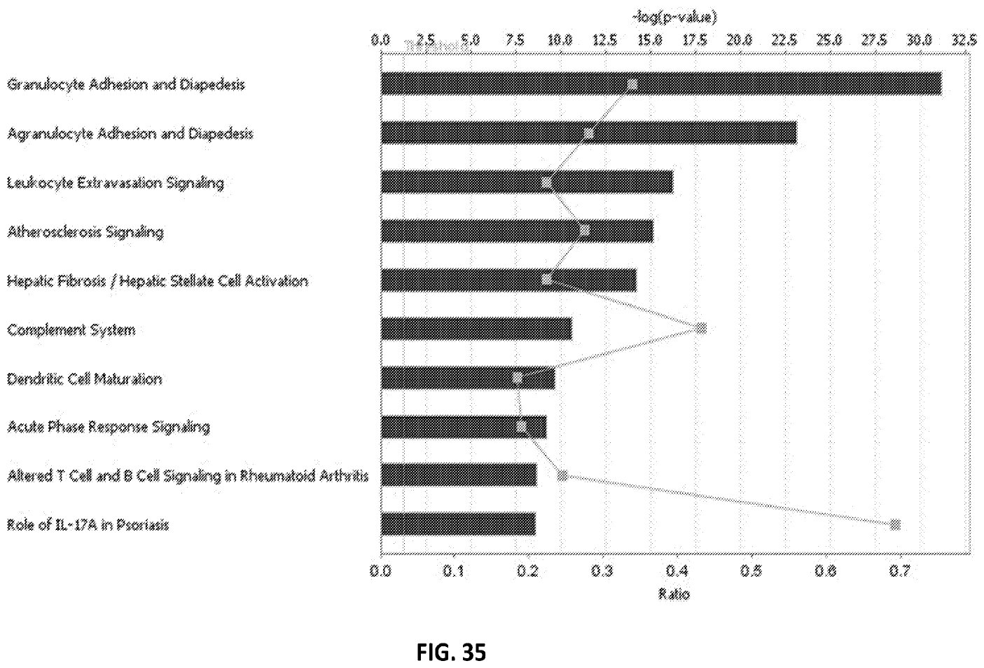

FIG. 35 shows a conserved response predicted downstream of IL-17A and IBD. One of the top 10 canonical pathways predicted to be involved in the UC/CD colon gene signature is the acute phase response signaling.

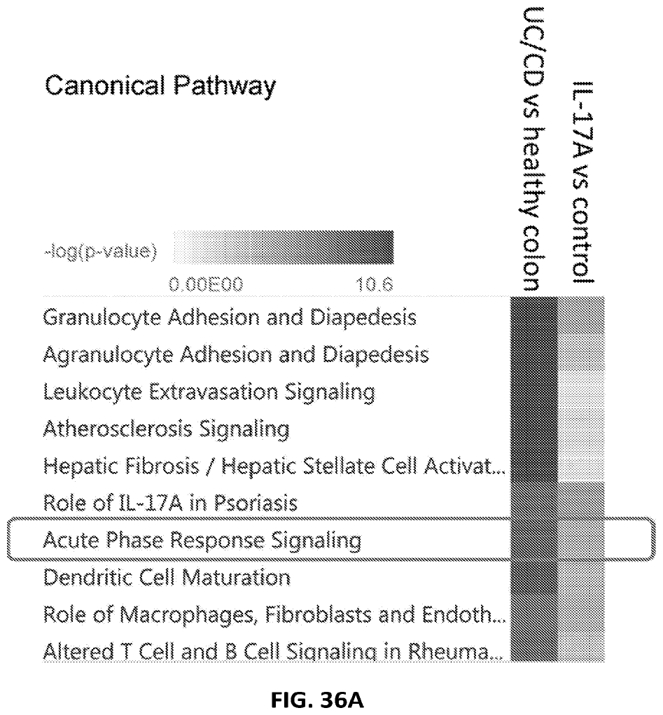

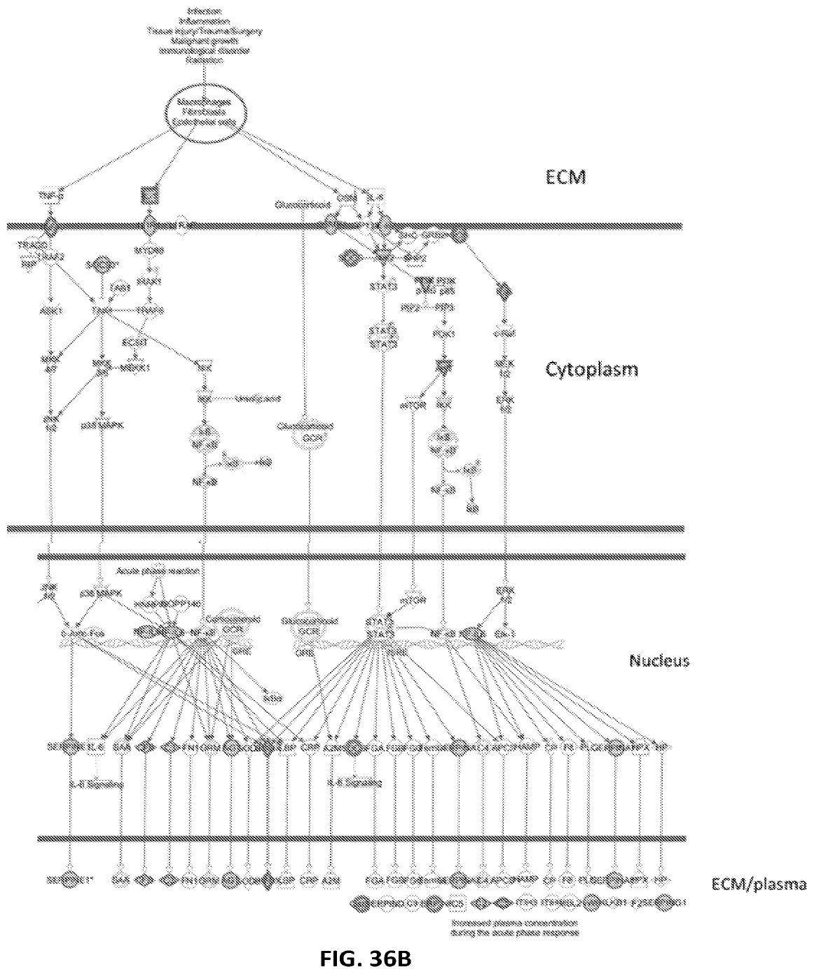

FIG. 36A. IPA comparative pathway analysis top 10 overlapping pathways of UC/CD and IL-17A treatment in vitro.

FIG. 36B. Acute phase response pathway. If we zoom in on this pathway we can see that it involves classic inflammatory mediators like TNF, IL-1, and IL-6, driving the activation of an acute response.

However, this includes our gene of interest Serpine1/PAI-1 and also many other closely related members of the serpin family highlighted here in purple (see e.g., FIG. 36B). Potentially what may occur in IBD is a state of acute inflammation in the colon drives the expression of PAI-1 and in susceptible individuals this PAI-1 process becomes chronically and highly elevated, which deregulates the mechanisms of immunosuppression mediated by TGFb.

As shown herein, the present disclosure has shown the discovery of PAI-1 (gene name Serpine1) as a biomarker of active inflammatory bowel disease and predictor of response to biologic therapy (i.e. anti-TNF therapy) using colon biopsies and/or plasma. The present disclosure has shown that (1) IBD can be diagnosed in a patient with IBD using PAI-1 levels; (2) they can predict treatment outcome based on PAI-1 levels; and (3) PAI-1 inhibitor (CDE-268, a known PAI-1 inhibitor to treat cardiac conditions) can successfully treat colitis.

We sought a marker of intestinal inflammation in IBD that is downstream of multiple inflammatory pathways. We first performed RNA microarray analysis on primary mouse intestinal epithelial cells treated these cells with IL-17 (a known important inflammatory cytokine in IBD). We cross-referenced a list of 23 molecules with enhanced mRNA production in these cells grown multiple states to lists of molecules with enhanced expression in IBD colon biopsies. We identified that the Plat/Serpine1 pathway was enriched. We found that Plat and Serpine1 mRNA is upregulated in IL-17-dominated intestinal models, such as DSS and Citrobacter rodentium infection. The elevation of Plat mRNA and protein (protein name tissue plasminogen activator; tPA) in the mouse models was functional as loss of function of tPA worsened disease and reduction in PAI-1 activity improved disease outcomes in multiple mouse models. PAI-1 is a direct binding inhibitor of Plat. We found that PAI-1 expression in disease models is elevated at the site of inflammation. Inhibition of PAI-1 elevates active Plat which rescues disease activity. PAI-1 protein expression was significantly elevated in immunofluorescence analysis of sections from ulcerative colitis (UC) resection cases as compared to similar UC sections without active disease as well as non-IBD cases (n=34 total samples). mRNA data from colon biopsies from 6 independent cohorts of ulcerative colitis and colonic Crohn's disease (CD) showed a significant increase in PAI-1 expression exclusively in patients with active disease as compared to inactive disease or non-IBD controls. In cohorts where biopsies were taken before and after treatment, we found that levels of PAI-1 predicted response to anti-TNF therapy (patients with high levels of PAI-1 were less likely to respond).

Plasma— PAI-1 protein levels are being tested in plasma to confirm the observations we have made in colon biopsies with mRNA also apply to protein levels in the blood. PAI-1 is readily detectable in the plasma and has been used as a biomarker of other diseases including cardiovascular disease. PAI-1 is unique in that it is induced downstream of multiple inflammatory factors linked to UC and CD and plasma levels correlate to levels in tissue in other disease states.

We have demonstrated the ability of PAI-1 expression levels to indicate disease activity in both ulcerative colitis and Crohn's disease. This has included analyzing the following patient specimens: 1. Resection cases (n=34) of patients will ulcerative colitis demonstrating increased PAI-1 in inflamed areas of colon. 2. Microarray analysis of >500 patients colon biopsies to show serpine1 expression predicts disease activity, and also using a smaller subset of patients to show that serpine1 expression can help predict whether or not a patient will respond to biologic therapy (e.g. anti-TNF therapy). 3. Plasma study pending to analyze the ability of PAI-1 protein levels in the blood (˜150 patients) to predict disease activity and response to biologic therapy (e.g. anti-TNF therapy).

### Abbreviations

tPA=tissue plasminogen activator; gene name Plat
PAI-1=plasminogen activator inhibitor 1; gene name Serpine1
UC=ulcerative colitis
CD=Crohn's disease

### Example 5: Additional Biomarkers to Increase Predictability of Therapeutic Response

Detecting additional biomarkers can increase the predictive power of treatment efficacy in patients with IBD. The following example describes gene expression signature to predict IBD responder vs. non-responder to anti-TNF therapy.

(I) 2-Way Biomarkers Tested (CCL2 and SERPINE1/PAI-1)

Below is data from showing the use of a 2 way biomarker (CCL2 and PAI-1/SERPINE) signature improves the prediction of biologic therapy responders vs. non-responders. Similar results were found for PAI-1/SERPINE, TNC, and IL13RA2.

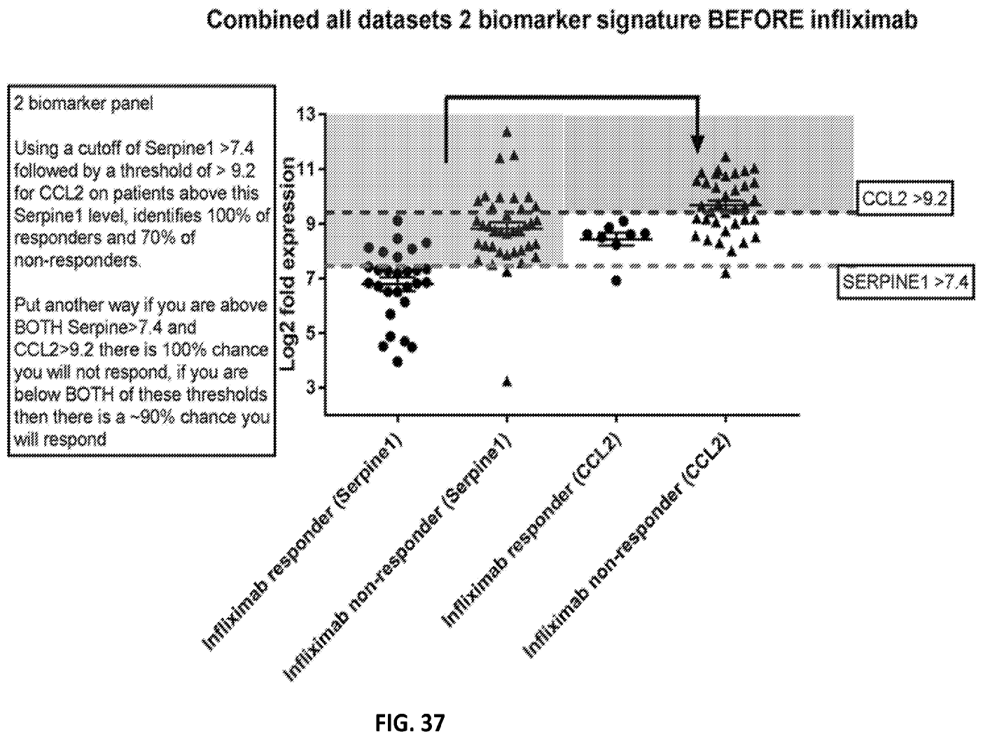

FIG. 37. Combined all data sets 2 biomarker signature.

(II) Sets of Multiple Biomarkers Tested

This example describes the biomarker results from a standard analysis and a higher powered analysis from a statistical collaborator. The analysis is from 3 cohorts, a total 66 patients.

1^{st} set.) The transcriptional signature from the results (lower statistical power than the 2^{nd} set, but gave preference to genes with greater fold changes between responders and non-responders, this was done because this is envisioned as a quantitative PCR assay on colon biopsies) is: SERPINE1, CCL2, TNC, and IL13RA2

2^{nd} set). From the random forest testing (gold standard for gene expression biomarker analysis very high powered statistics but does not specify a high or low fold change) the final signature is: PRNP, IL13RA2, GPX8, DRAM1, and STAT4.

The ROC curve AUC for this reaches a 96% sensitivity and 97% specificity for predicting which IBD patients will go on to either RESPOND or NOT-RESPOND to anti-TNFa.

The 2^{nd} set is currently more statistically robust than the first, but when this method is developed into a PCR-based test on pre-collected cohorts, the fold change on the 2^{nd} gene set is much lower than the 1^{st} set, even if it predicts a higher % of patients. So assay-wise, the 1^{st} set may prove better.

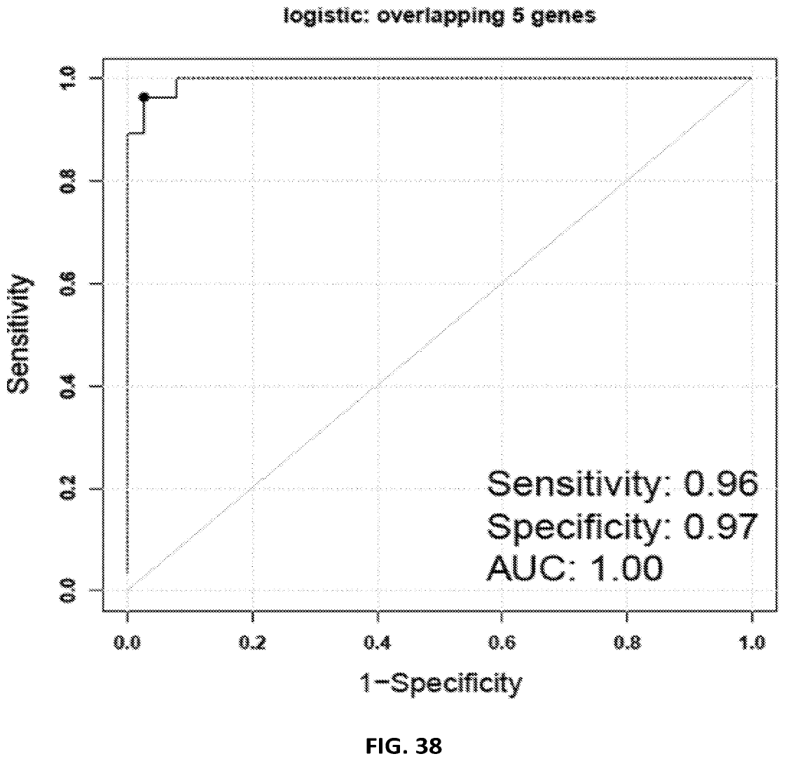

FIG. 38 shows data for the 5 biomarker signature showing diagnostic predictive power to discriminate responders vs non-responders to anti-TNFa.

TABLE 2
Frequency summary of samples by cohort and responder

| | Non-responder | Responder | Row Total |
|---|---|---|---|
| Cohort 1 | 16 | 8 | 24 |
| Cohort 2 | 7 | 12 | 19 |
| Cohort 3 | 15 | 8 | 23 |
| Column Total | 38 | 28 | 66 |

The gene expression data of cohort1 and 2 was merged with the gene expression data of cohort 3. Cohort 1 and Cohort 2, both profiled on Affymetrix Hgu133 plus, were normalized together from the raw data and collapsed to unique genes by mean (by Gerard), containing a total of 23520 genes. Cohort 3, on Hgu1.0ST version 1, was normalized separately from cohort 1 and 2 and collapsed to gene by means (by Gerard), containing 20475 genes. 17272 genes overlapped between the two normalized gene data sets (data not shown).

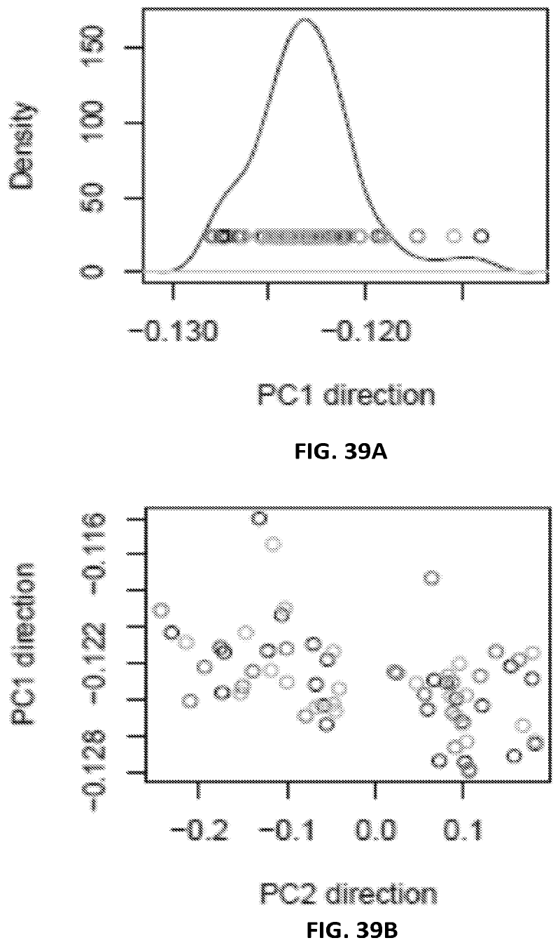

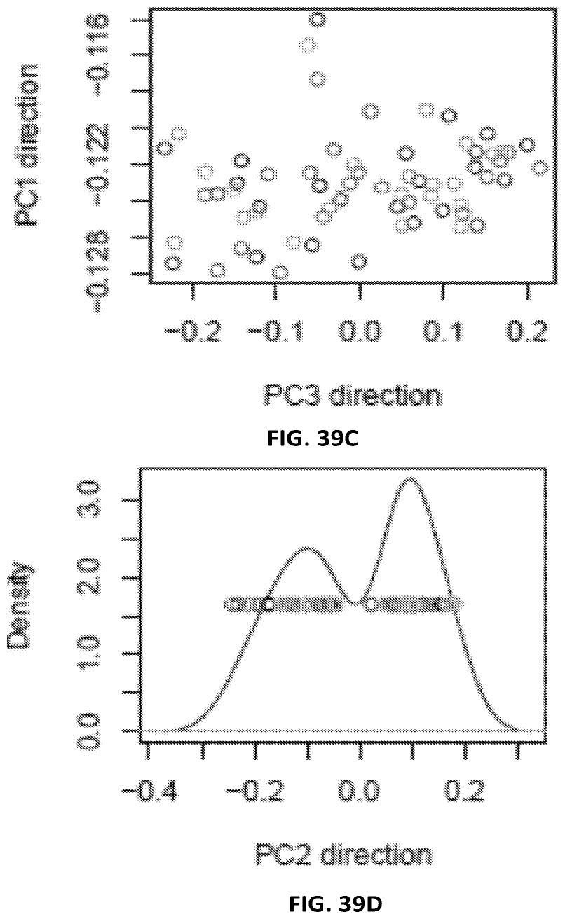

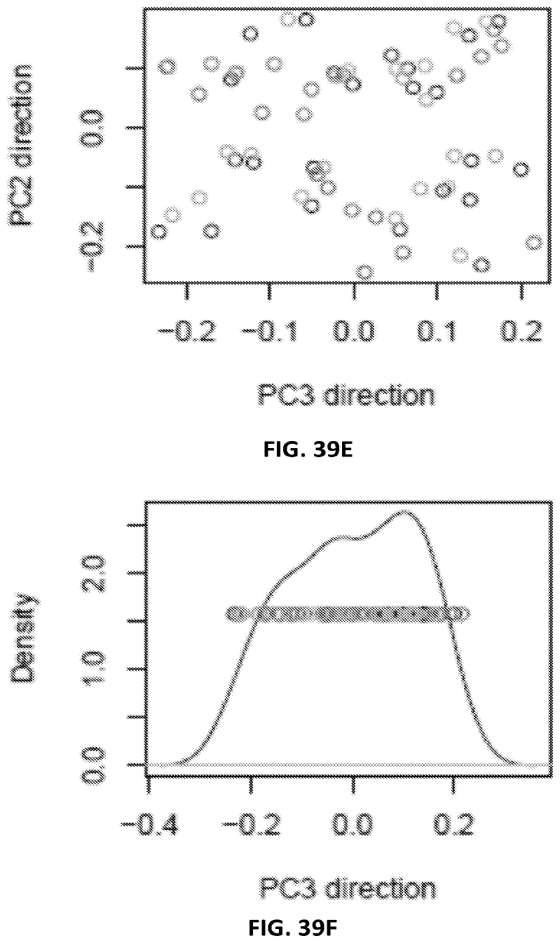

The gene expression data of cohort 1, 2, and 3 were merged together while removing batch effects using the COMBAT method [reference] as implemented in the Bioconductor package “sva” [reference]. The principal components (PC) analysis plots the density of the first three PCs (PC1, PC2, PC3) at diagonal and pairwise scatter plots between them. The black, red and green colored points indicate individual patient samples from cohort 1, 2, 3 respectively. Cohort 3 samples mingled well with cohort 1 & 2 samples based on the first 3 PCs. (The PCA plot is FIG. 39).

Heatmap on the merged gene expression matrix was generated with the patient samples at column and genes at rows, each clustered via the hierarchical clustering method with an averaged linkage and based on similarity gauged by Pearson correlation coefficient. The heatmap also indicates batch effect was negligible in patient samples.

The supervised classification method, random forest (RF), was used to classify responders vs. non-responders. RF is a tree-based machine learning classification algorithm using the resampling technique. RF repeatedly and randomly draw a set of samples of the original data of the same size as the original samples (here, 66 samples). The resampled data is used to build an ensemble of trees (here, 5000 trees) to classify patient samples into responder vs. non-responders. Each tree is allowed to have a maximum number of terminal nodes (here, 5) and at each tree branch split, multiple trials (here, 10) were performed to select the best splitting genes. The left-out samples are then predicted by the majority vote of the ensemble trees established by RF. The classification error rate can then finally be robustly evaluated by tabulating the true status and the predicted status. Moreover, several importance measures will be reported on each gene by evaluating the mean decrease in gini index (a purity measure of tree nodes) and overall classification accuracy after permuting the gene only (while keeping the other genes untouched).

TABLE 3
The individual true status and predicted status on each
of the 66 samples are provided in the table below.

| PID | RF.predicted.status | true.status |
|---|---|---|
| GSM364633 | Infliximab responder BEFORE | Infliximab responder BEFORE |
| GSM364634 | Infliximab responder BEFORE | Infliximab responder BEFORE |
| GSM364635 | Infliximab responder BEFORE | Infliximab responder BEFORE |
| GSM364636 | Infliximab responder BEFORE | Infliximab responder BEFORE |
| GSM364637 | Infliximab responder BEFORE | Infliximab responder BEFORE |
| GSM364638 | Infliximab responder BEFORE | Infliximab responder BEFORE |
| GSM364639 | Infliximab responder BEFORE | Infliximab responder BEFORE |
| GSM364640 | Infliximab responder BEFORE | Infliximab responder BEFORE |
| GSM364641 | Infliximab non-responder BEFORE | Infliximab non-responder BEFORE |
| GSM364642 | Infliximab non-responder BEFORE | Infliximab non-responder BEFORE |
| GSM364643 | Infliximab non-responder BEFORE | Infliximab non-responder BEFORE |
| GSM364644 | Infliximab non-responder BEFORE | Infliximab non-responder BEFORE |
| GSM364645 | Infliximab non-responder BEFORE | Infliximab non-responder BEFORE |
| GSM364646 | Infliximab non-responder BEFORE | Infliximab non-responder BEFORE |
| GSM364647 | Infliximab non-responder BEFORE | Infliximab non-responder BEFORE |
| GSM364648 | Infliximab non-responder BEFORE | Infliximab non-responder BEFORE |
| GSM364649 | Infliximab non-responder BEFORE | Infliximab non-responder BEFORE |
| GSM364650 | Infliximab non-responder BEFORE | Infliximab non-responder BEFORE |
| GSM364651 | Infliximab non-responder BEFORE | Infliximab non-responder BEFORE |
| GSM364652 | Infliximab non-responder BEFORE | Infliximab non-responder BEFORE |
| GSM364653 | Infliximab non-responder BEFORE | Infliximab non-responder BEFORE |
| GSM364654 | Infliximab non-responder BEFORE | Infliximab non-responder BEFORE |
| GSM364655 | Infliximab non-responder BEFORE | Infliximab non-responder BEFORE |
| GSM364656 | Infliximab non-responder BEFORE | Infliximab non-responder BEFORE |
| GSM423010 | Infliximab responder BEFORE | Infliximab responder BEFORE |
| GSM423012 | Infliximab responder BEFORE | Infliximab responder BEFORE |
| GSM423013 | Infliximab responder BEFORE | Infliximab responder BEFORE |
| GSM423015 | Infliximab responder BEFORE | Infliximab responder BEFORE |
| GSM423017 | Infliximab responder BEFORE | Infliximab responder BEFORE |
| GSM423019 | Infliximab responder BEFORE | Infliximab responder BEFORE |
| GSM423021 | Infliximab responder BEFORE | Infliximab responder BEFORE |
| GSM423023 | Infliximab responder BEFORE | Infliximab responder BEFORE |
| GSM423025 | Infliximab responder BEFORE | Infliximab responder BEFORE |
| GSM423027 | Infliximab responder BEFORE | Infliximab responder BEFORE |
| GSM423029 | Infliximab responder BEFORE | Infliximab responder BEFORE |
| GSM423031 | Infliximab responder BEFORE | Infliximab responder BEFORE |
| GSM423033 | Infliximab non-responder BEFORE | Infliximab non-responder BEFORE |
| GSM423035 | Infliximab non-responder BEFORE | Infliximab non-responder BEFORE |
| GSM423037 | Infliximab non-responder BEFORE | Infliximab non-responder BEFORE |
| GSM423039 | Infliximab non-responder BEFORE | Infliximab non-responder BEFORE |
| GSM423041 | Infliximab non-responder BEFORE | Infliximab non-responder BEFORE |
| GSM423043 | Infliximab non-responder BEFORE | Infliximab non-responder BEFORE |
| GSM423045 | Infliximab non-responder BEFORE | Infliximab non-responder BEFORE |
| GSM1900148 | Infliximab non-responder BEFORE | Infliximab non-responder BEFORE |
| GSM1900154 | Infliximab non-responder BEFORE | Infliximab non-responder BEFORE |
| GSM1900155 | Infliximab non-responder BEFORE | Infliximab non-responder BEFORE |
| GSM1900158 | Infliximab responder BEFORE | Infliximab responder BEFORE |
| GSM1900172 | Infliximab responder BEFORE | Infliximab responder BEFORE |
| GSM1900175 | Infliximab non-responder BEFORE | Infliximab non-responder BEFORE |
| GSM1900176 | Infliximab non-responder BEFORE | Infliximab non-responder BEFORE |
| GSM1900180 | Infliximab responder BEFORE | Infliximab responder BEFORE |
| GSM1900181 | Infliximab non-responder BEFORE | Infliximab non-responder BEFORE |
| GSM1900184 | Infliximab non-responder BEFORE | Infliximab non-responder BEFORE |
| GSM1900185 | Infliximab non-responder BEFORE | Infliximab non-responder BEFORE |
| GSM1900186 | Infliximab responder BEFORE | Infliximab responder BEFORE |
| GSM1900192 | Infliximab responder BEFORE | Infliximab responder BEFORE |
| GSM1900195 | Infliximab non-responder BEFORE | Infliximab non-responder BEFORE |
| GSM1900202 | Infliximab non-responder BEFORE | Infliximab non-responder BEFORE |
| GSM1900204 | Infliximab responder BEFORE | Infliximab responder BEFORE |
| GSM1900206 | Infliximab non-responder BEFORE | Infliximab non-responder BEFORE |
| GSM1900208 | Infliximab non-responder BEFORE | Infliximab non-responder BEFORE |
| GSM1900210 | Infliximab responder BEFORE | Infliximab responder BEFORE |
| GSM1900213 | Infliximab responder BEFORE | Infliximab responder BEFORE |
| GSM1900214 | Infliximab non-responder BEFORE | Infliximab non-responder BEFORE |
| GSM1900215 | Infliximab non-responder BEFORE | Infliximab non-responder BEFORE |
| GSM1900217 | Infliximab non-responder BEFORE | Infliximab non-responder BEFORE |

Classification Error Matrix.

Due to the random sampling nature of the algorithm, the confusion matrix may vary slightly from run to run. Among the 66 patient samples, 42 were predicted to be non-responder, 24 as responders. 33 out of the 38 true non-responders were predicted correctly while only 19 out of the 28 true responders were predicted as responders, corresponding to a class error rate of 13.16% in true non-responders and 32.14% in true responders. The overall classification accuracy is (34+19)/66=80.30%.

TABLE 4

| | Total | | | |
|---|---|---|---|---|
| True | samples | Predicted | | Class |
| responder | with true | Non- | Predicted | classification |
| status | status | responder | Responder | error |
| Non-Responder | 38 | 34 | 4 | 0.105263 |
| Responder | 28 | 9 | 19 | 0.3214 |
| Total samples | 66 | 43 | 23 | |
| with predicted | | | | |
| status | | | | |

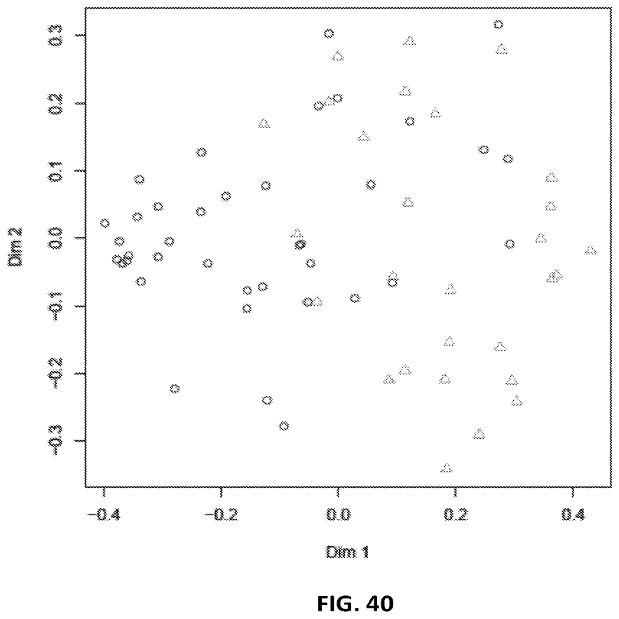

The multi-dimensional scaling (MDS, a dimension reduction technique similar to PCA) plot is used to visualize the proximity of the samples on original high dimension on a 2-dimensional plane (MDS dimension 1 vs. MDS dimension 2) with the non-responders in black circle and the responders in green triangle (see e.g., FIG. 40).

The importance measures of all genes sorted by mean Gini index decrease. Due to the random sampling nature of the algorithm, the ordering of the genes may change from run to run but the overall importance (e.g., within top 100 should be stable, data not shown).

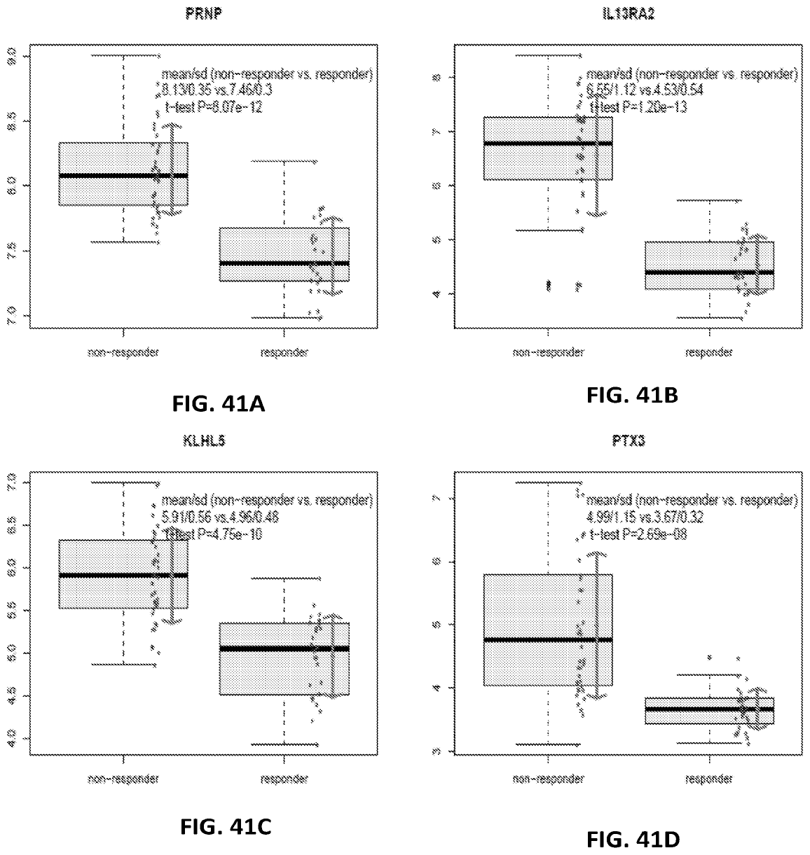

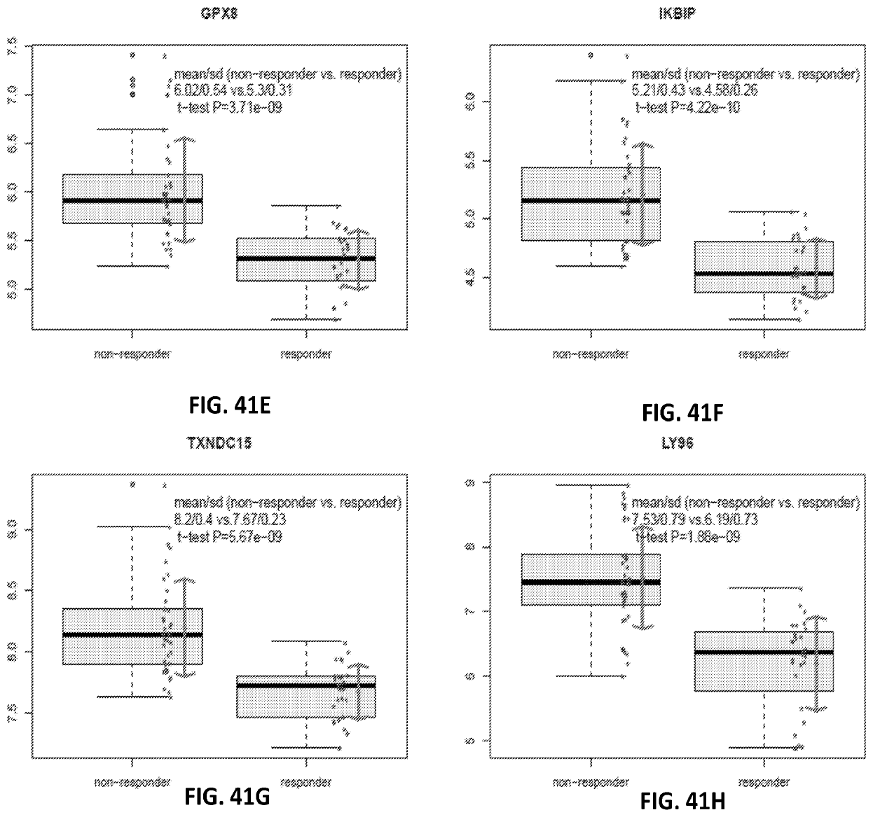

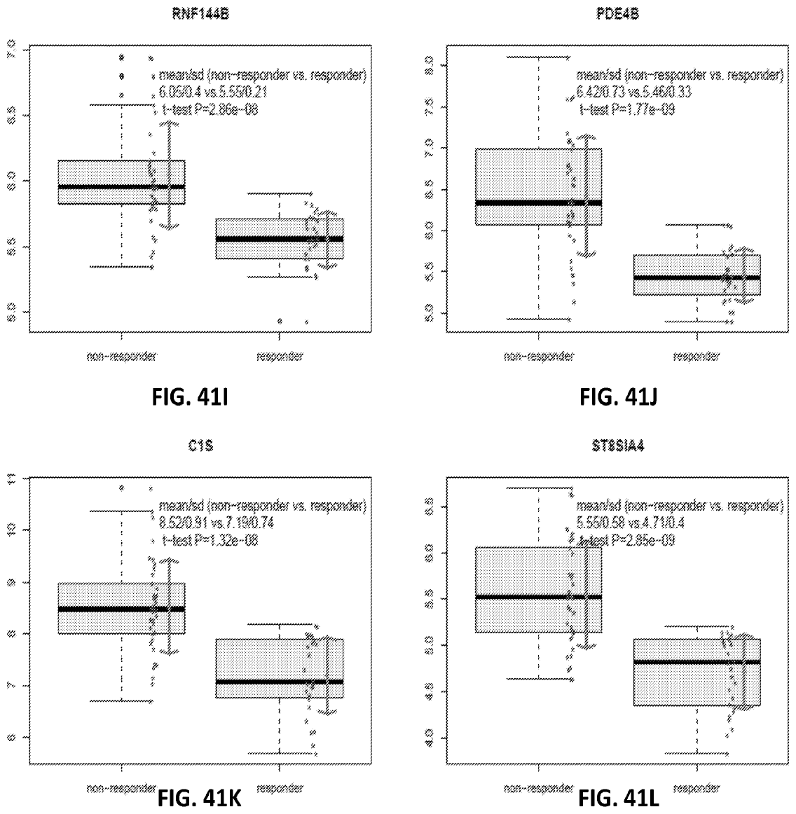

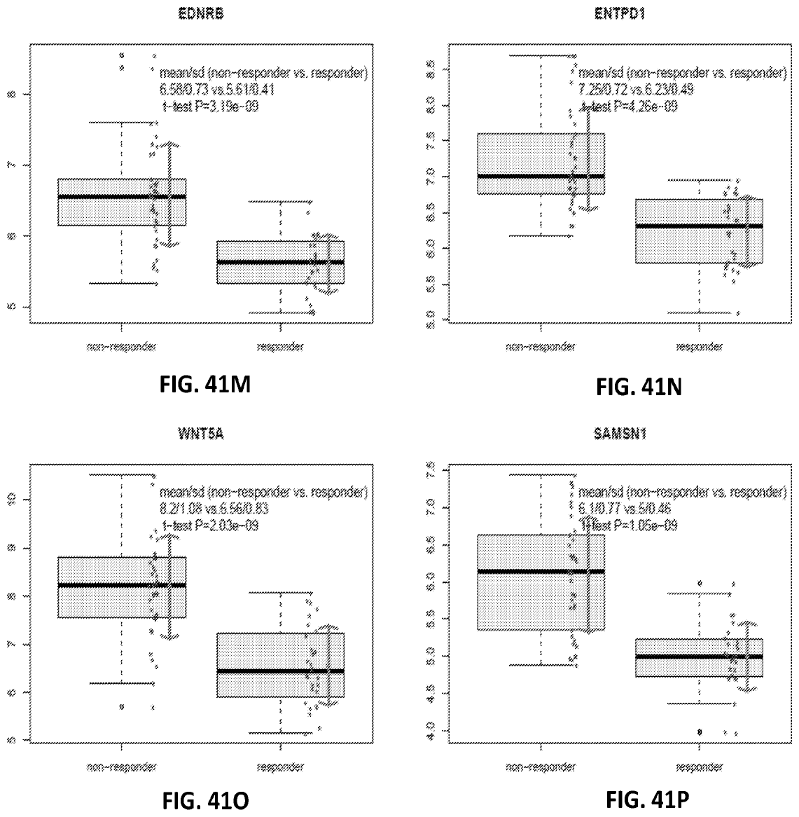

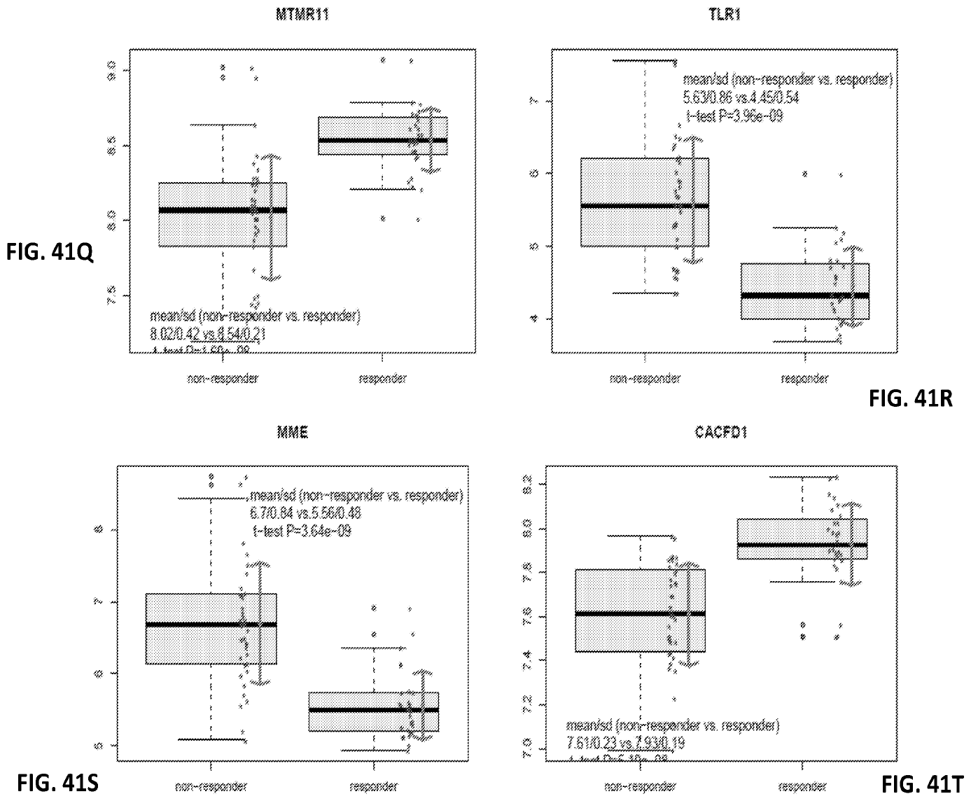

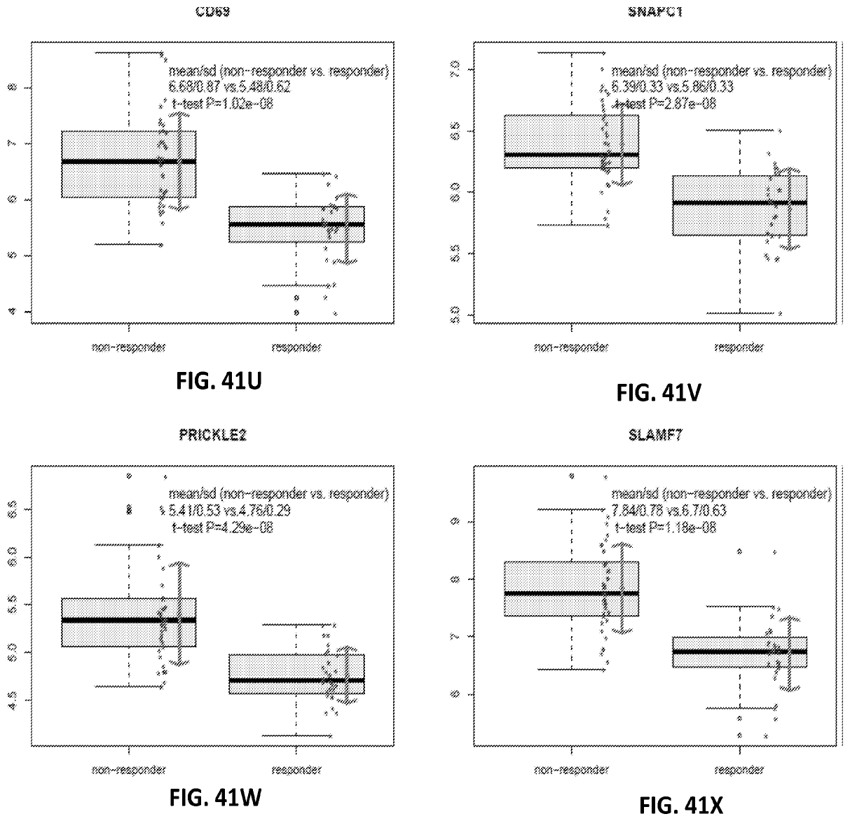

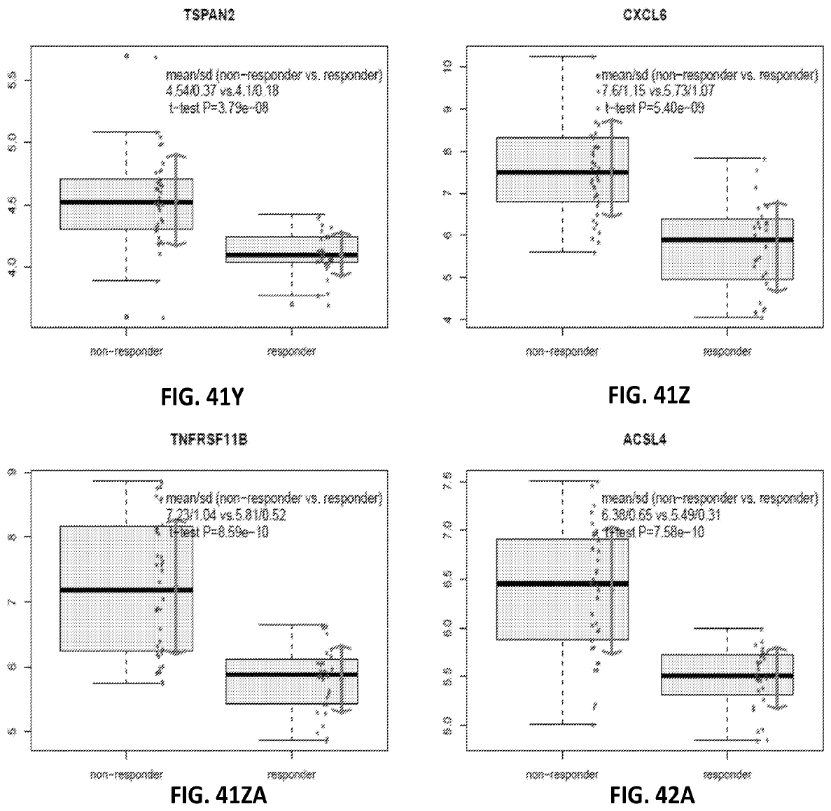

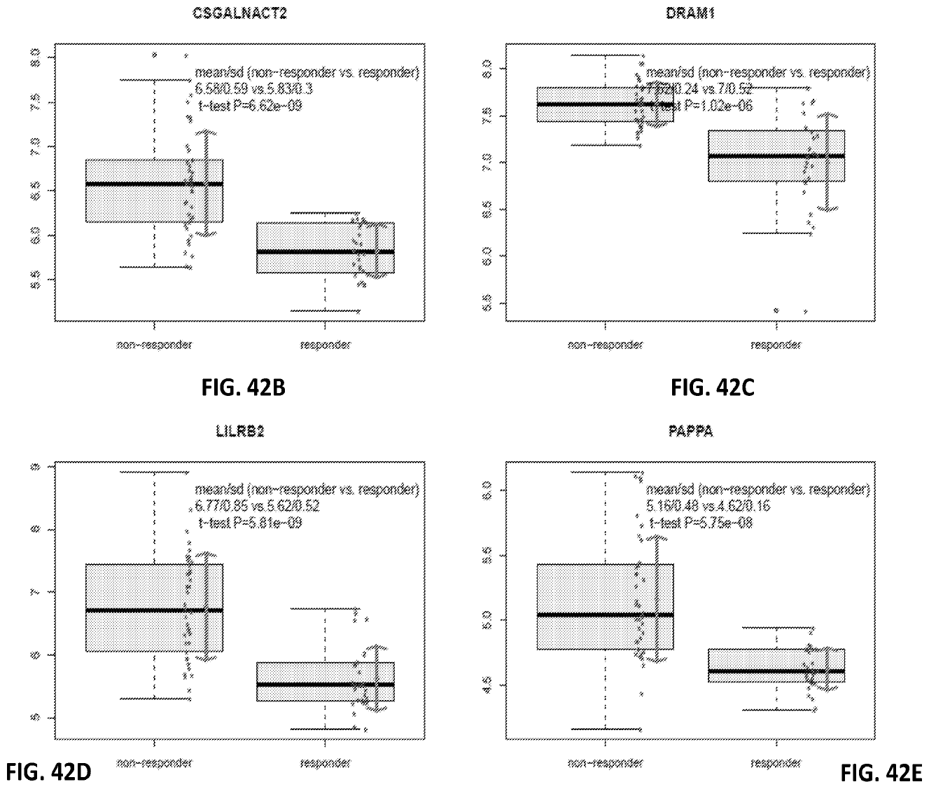

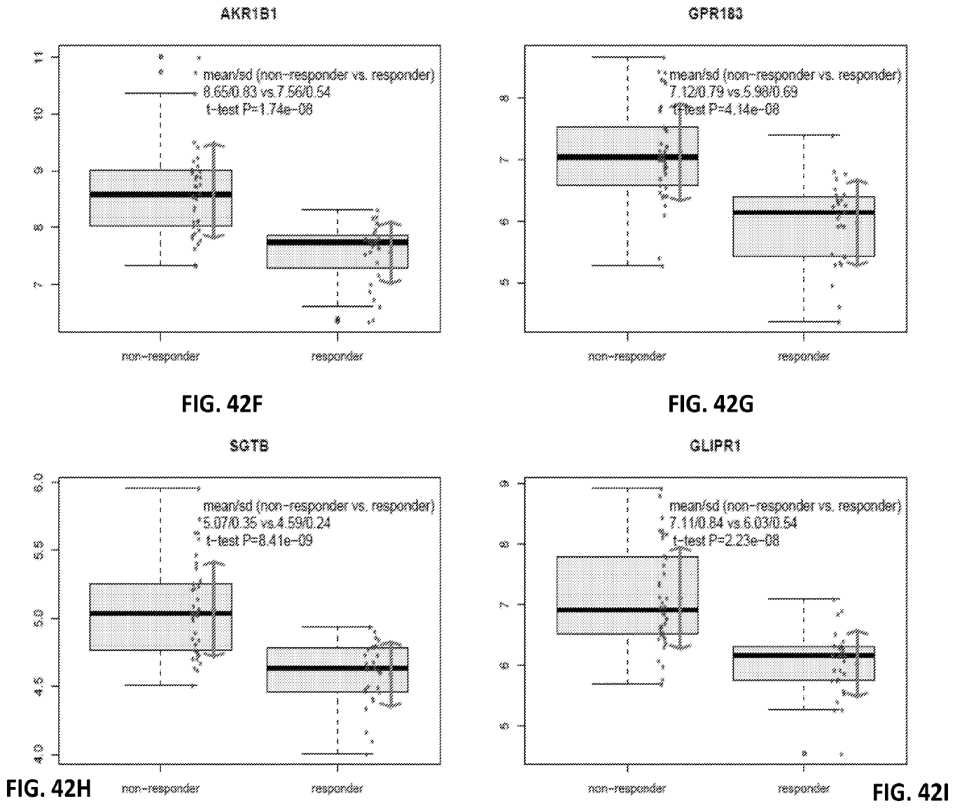

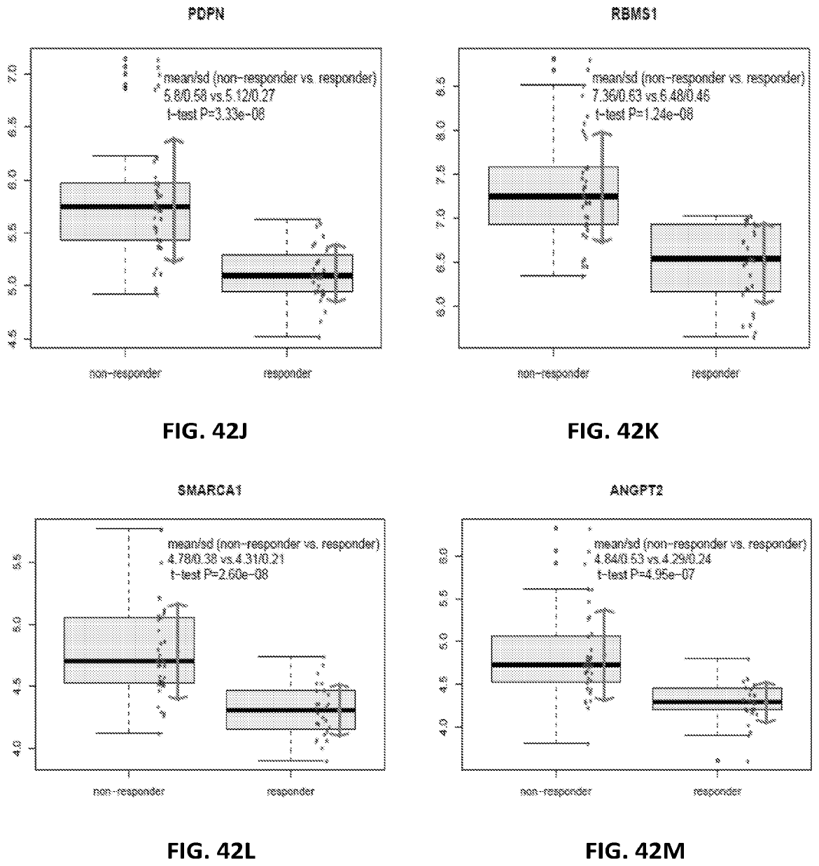

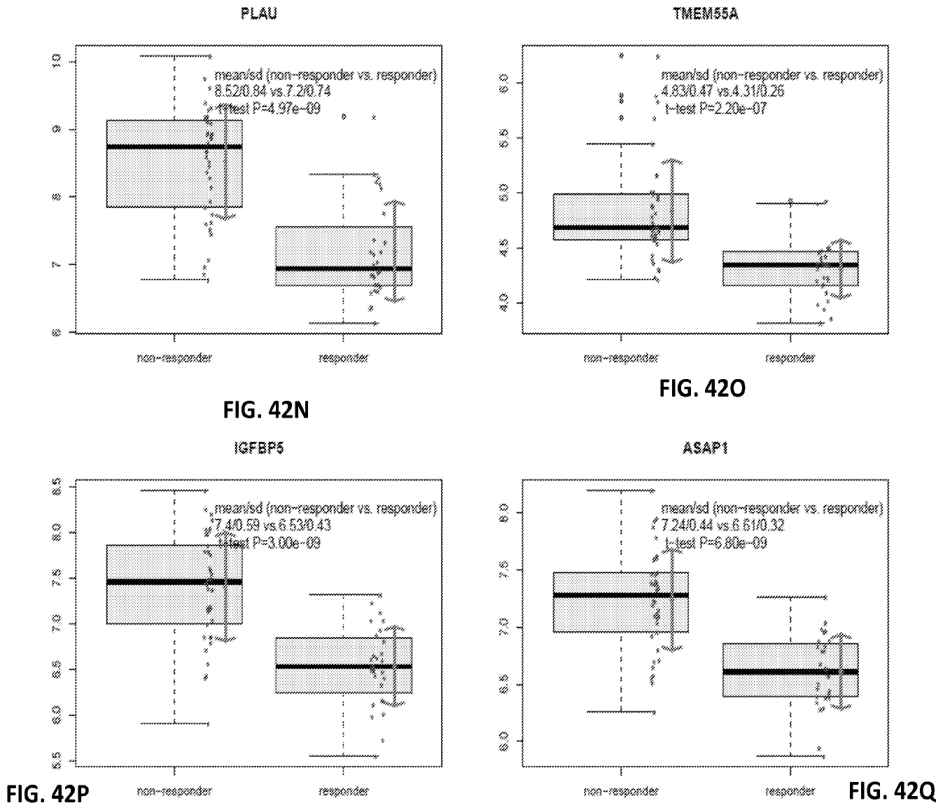

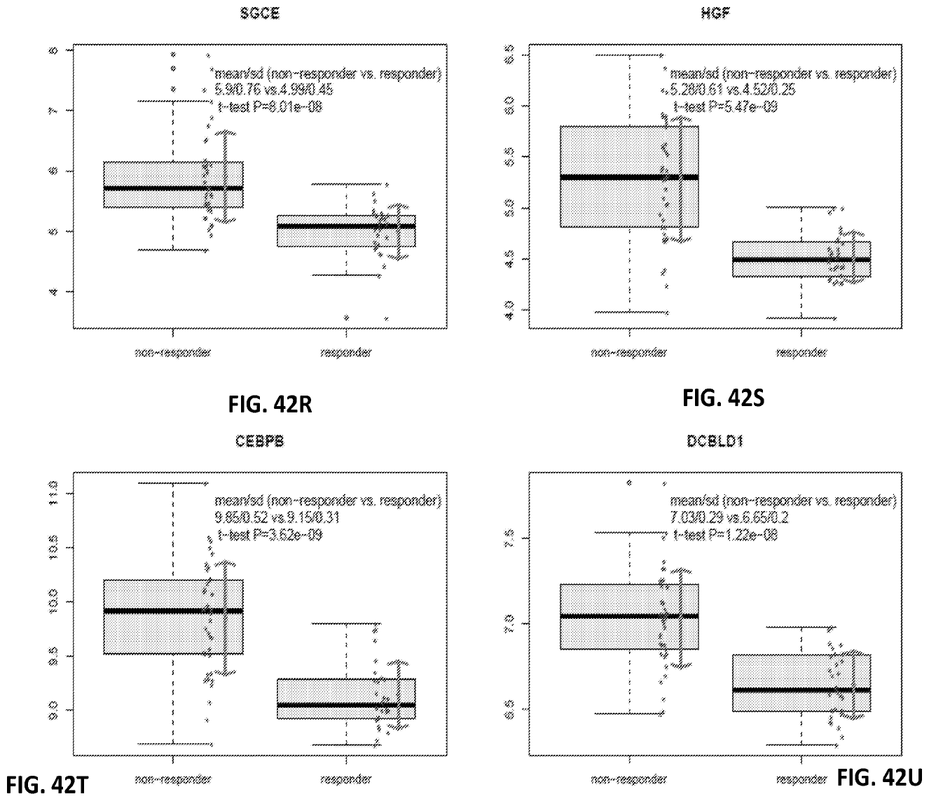

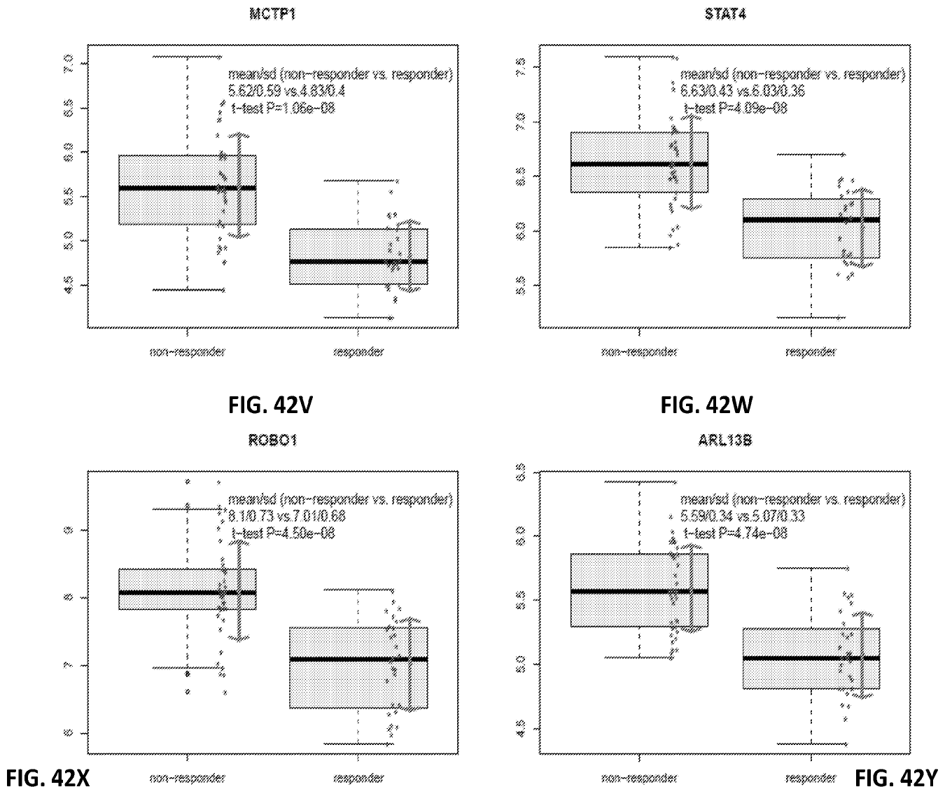

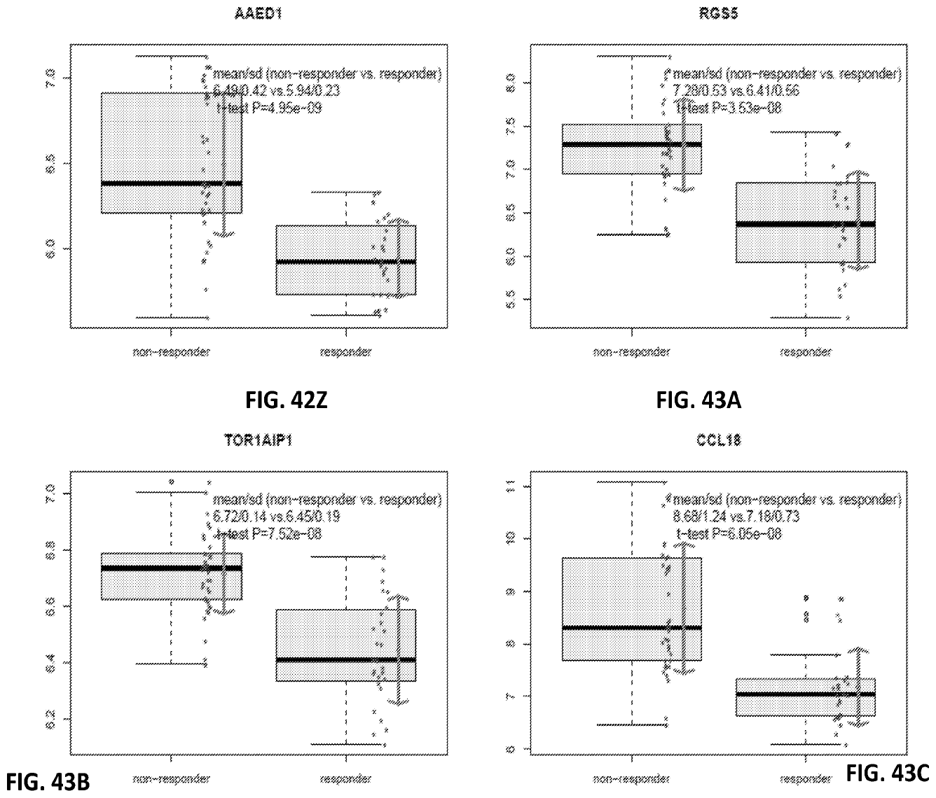

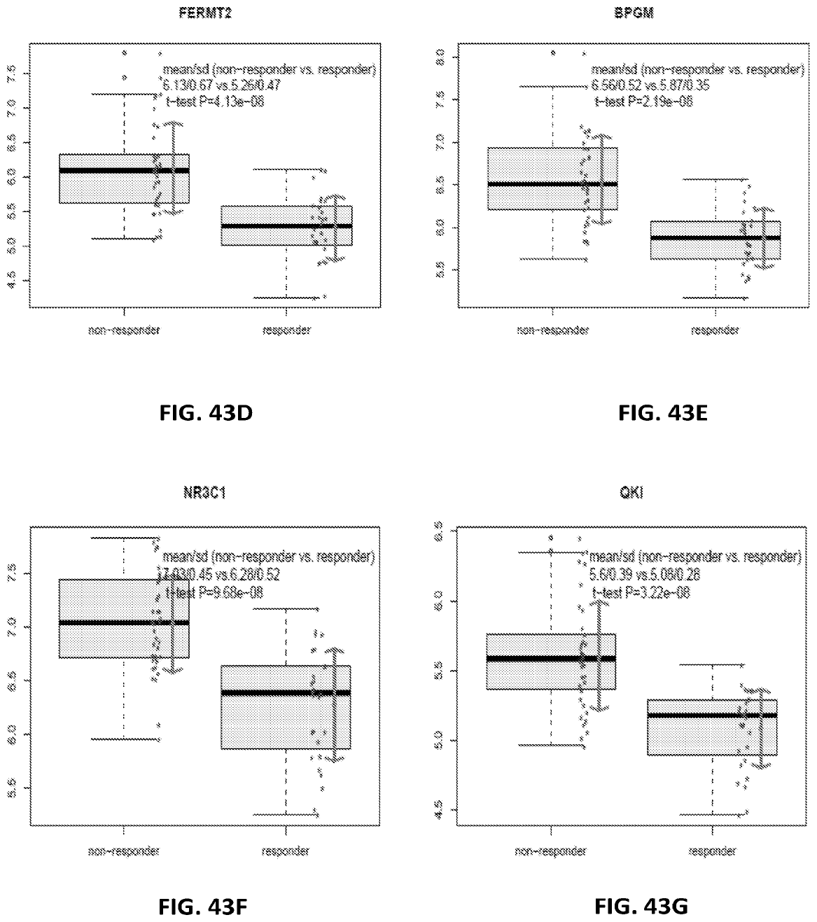

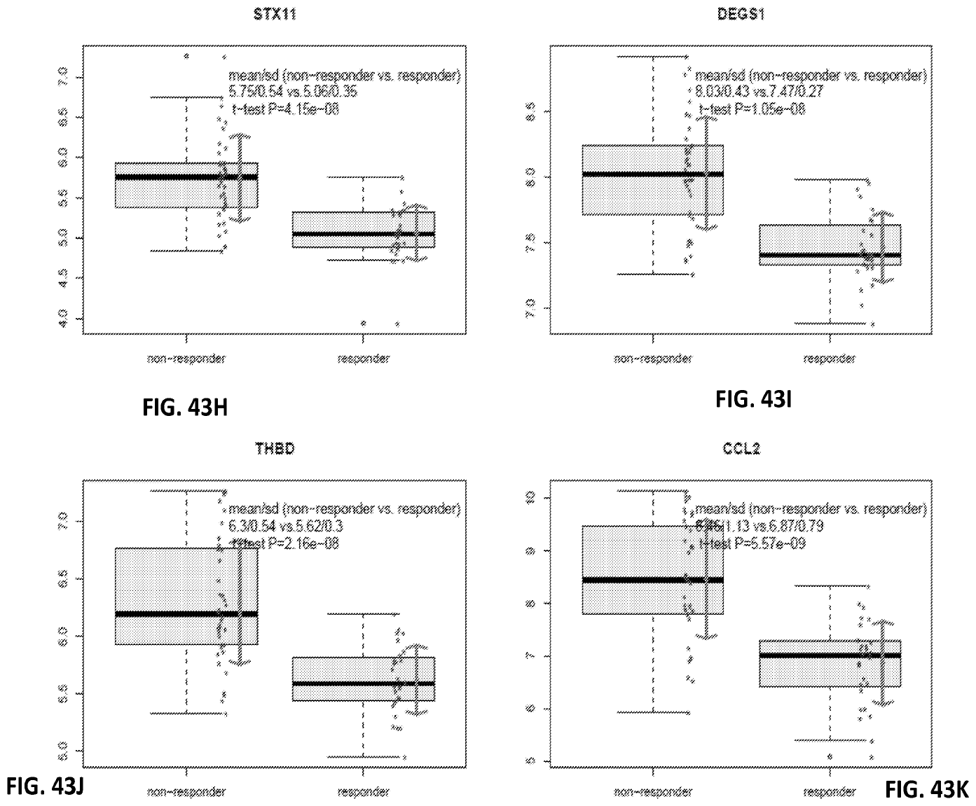

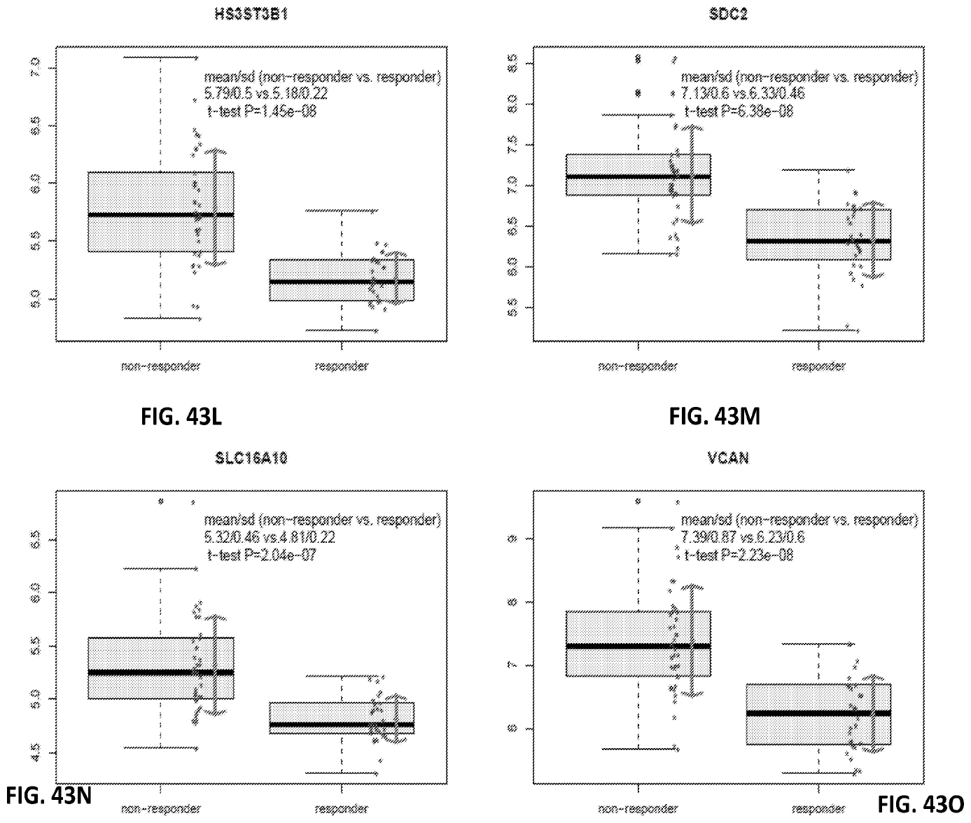

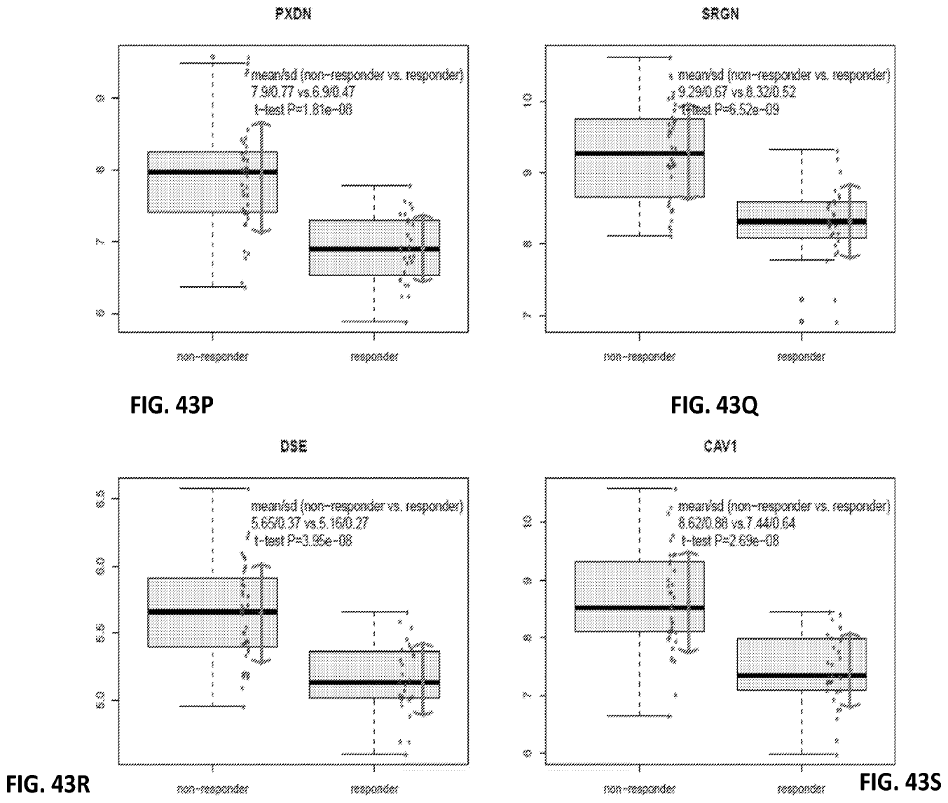

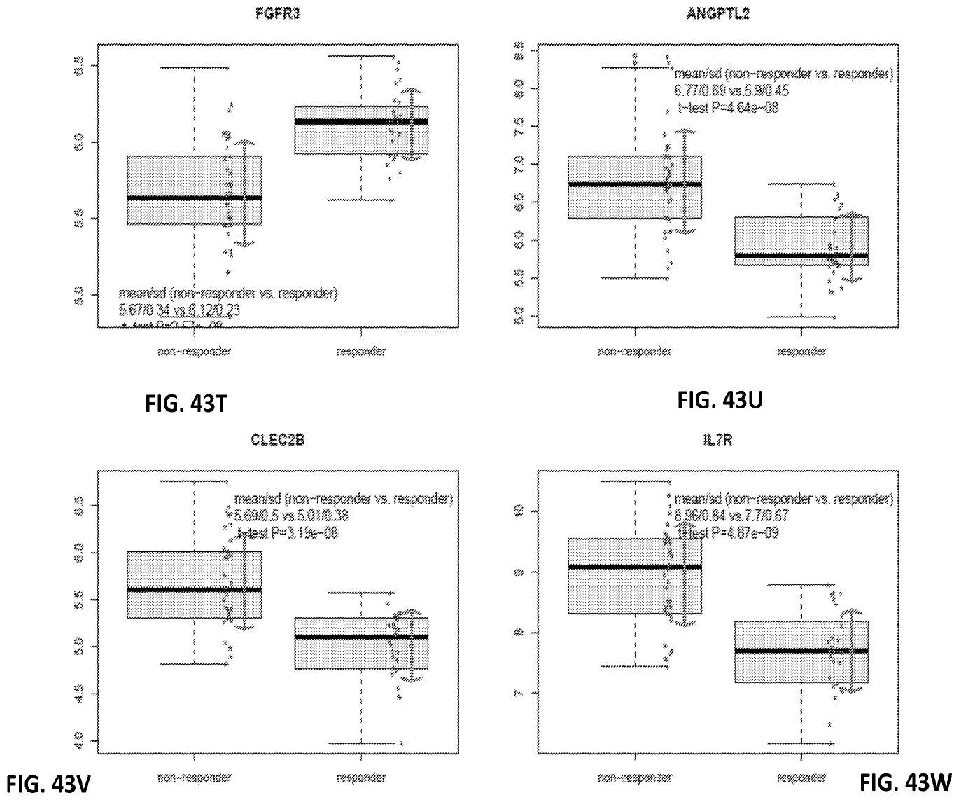

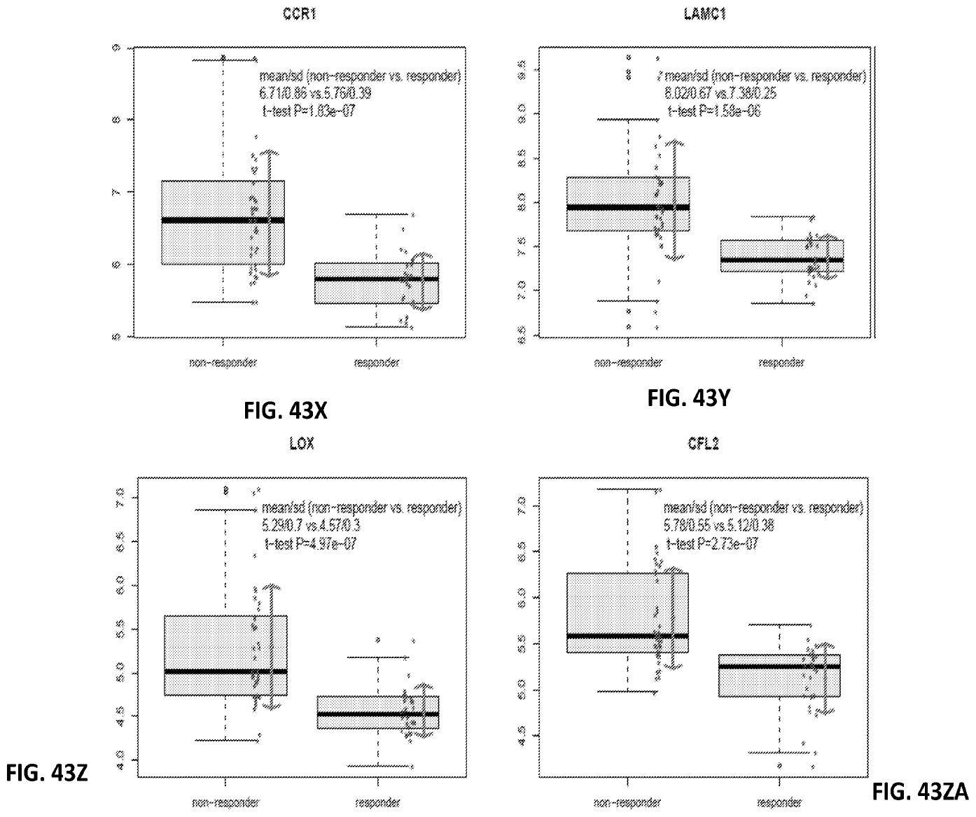

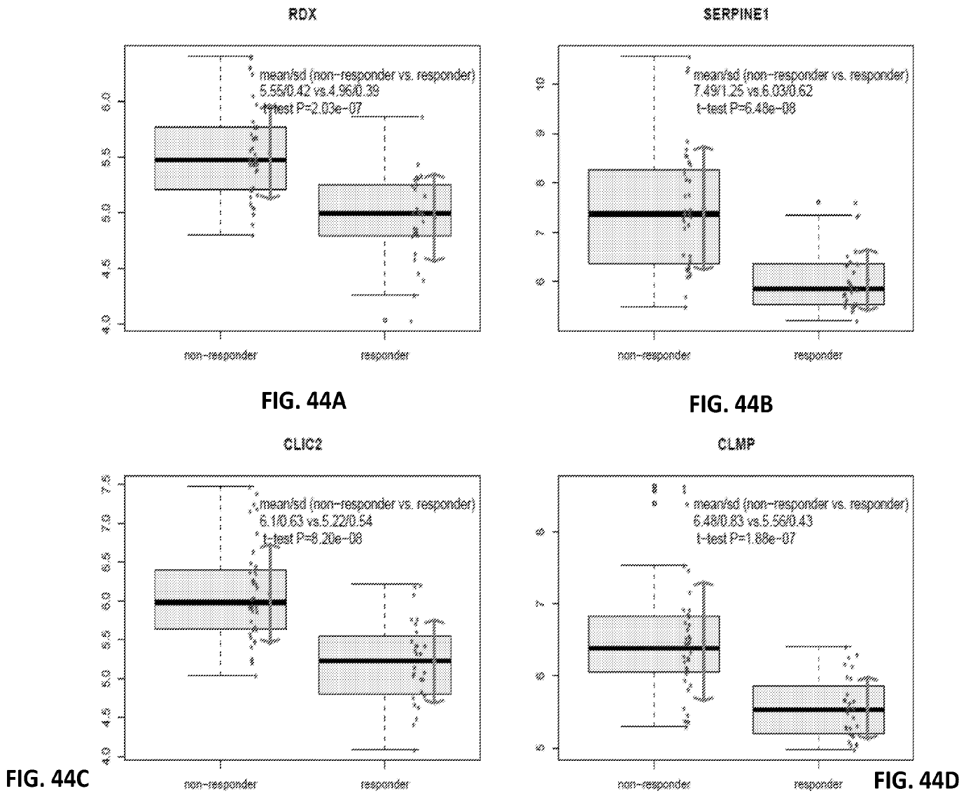

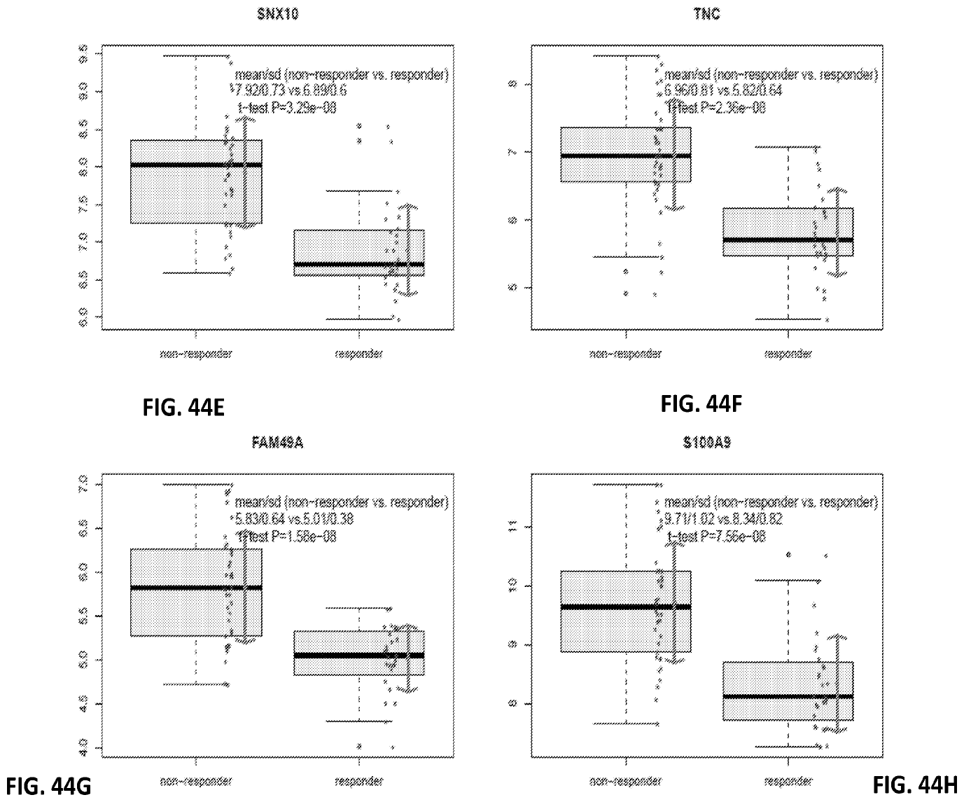

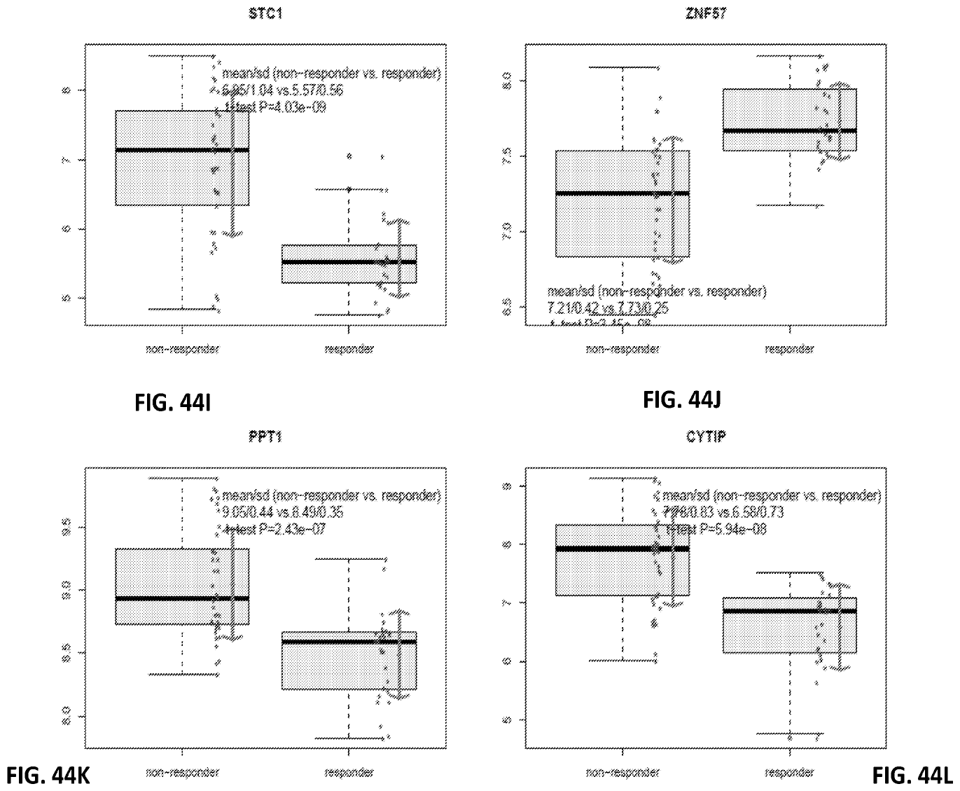

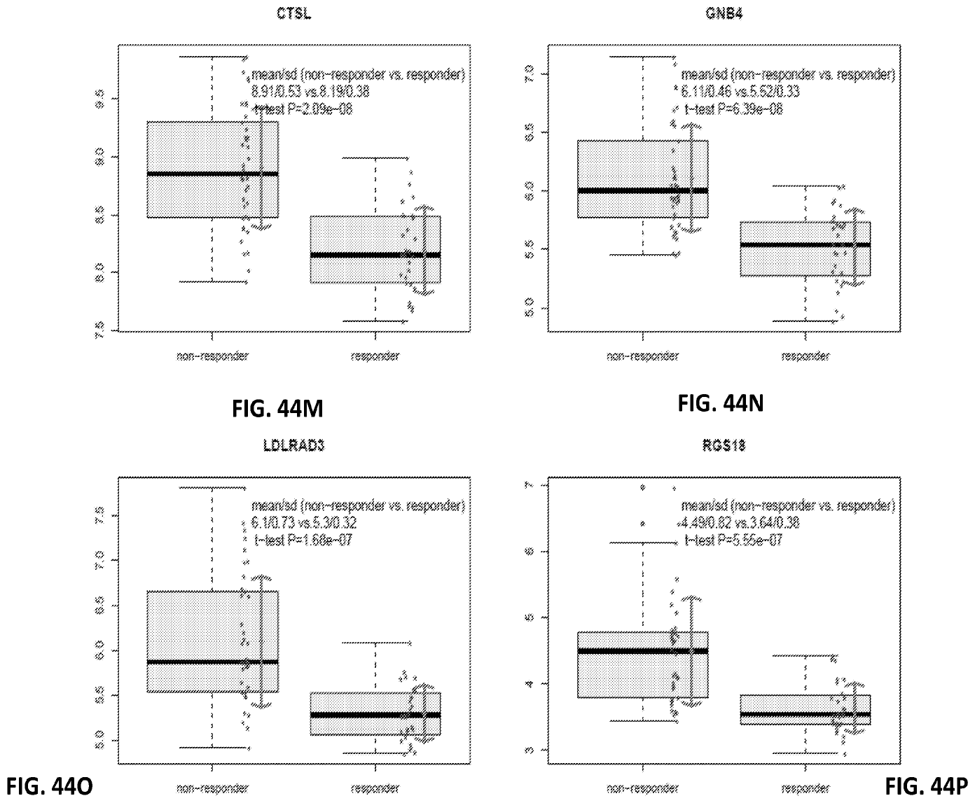

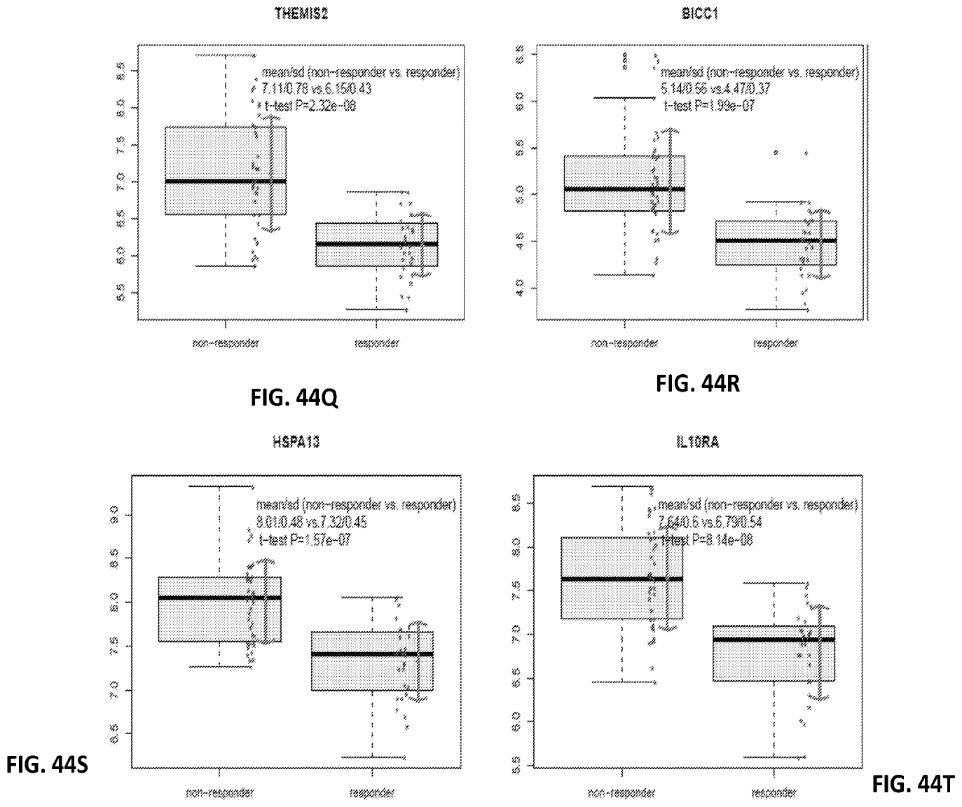

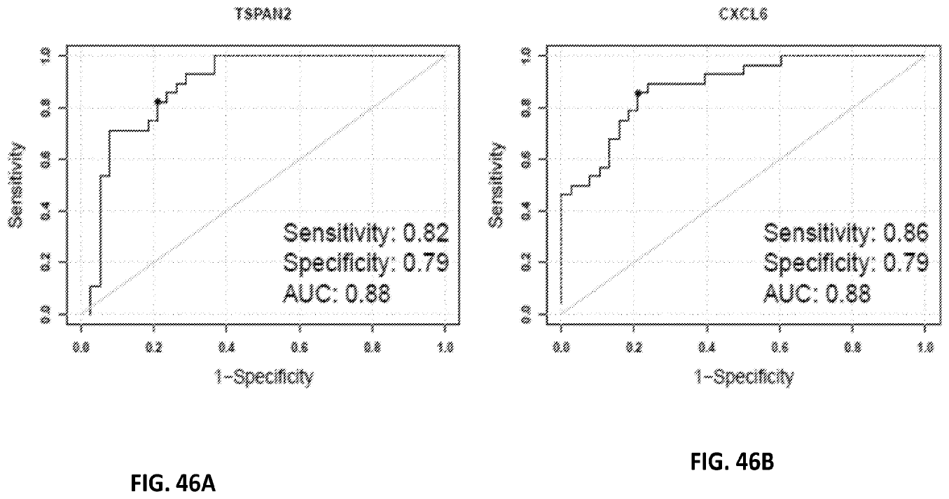

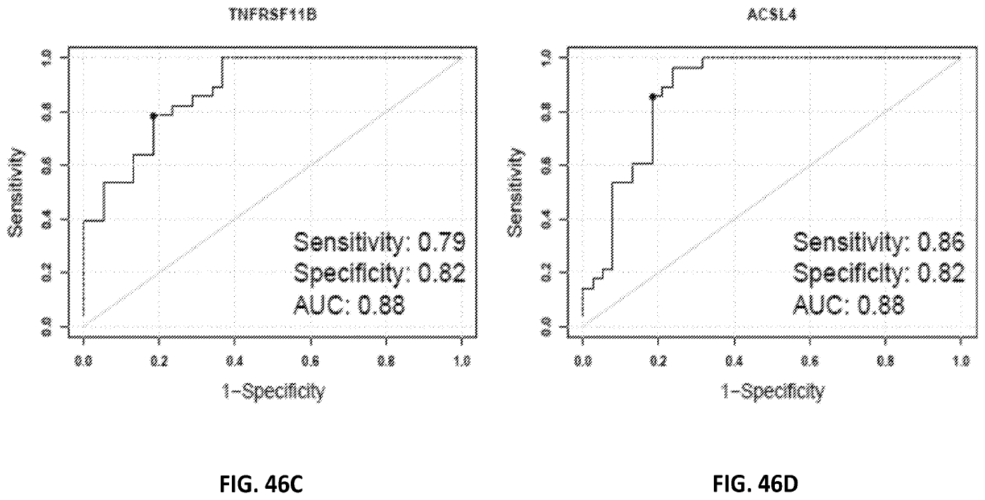

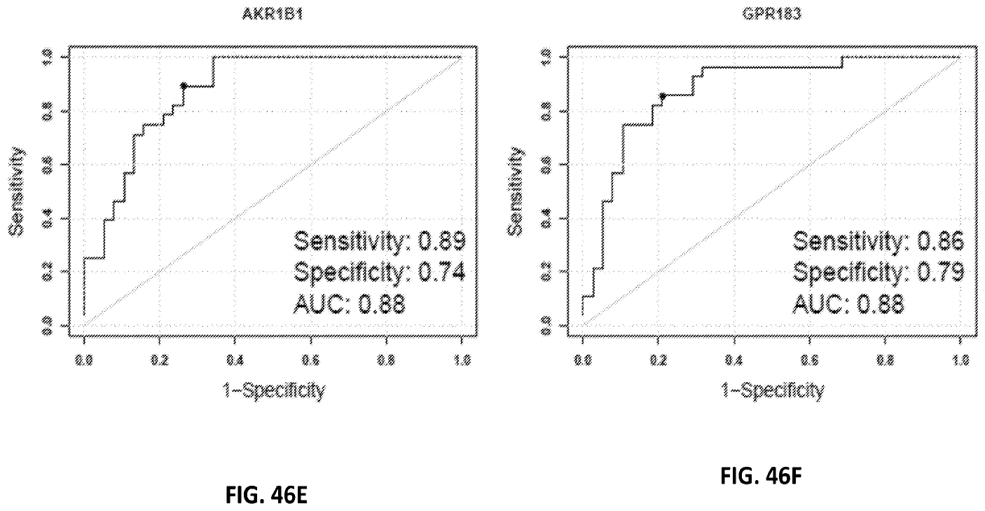

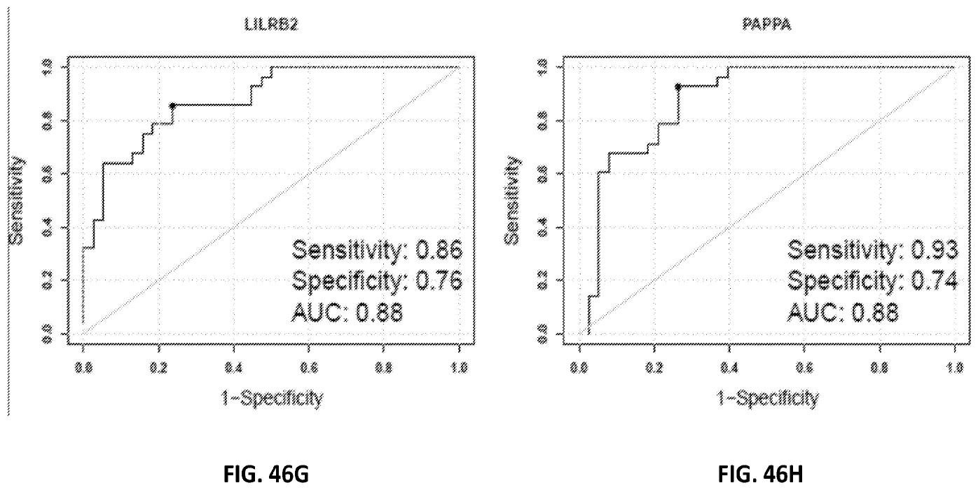

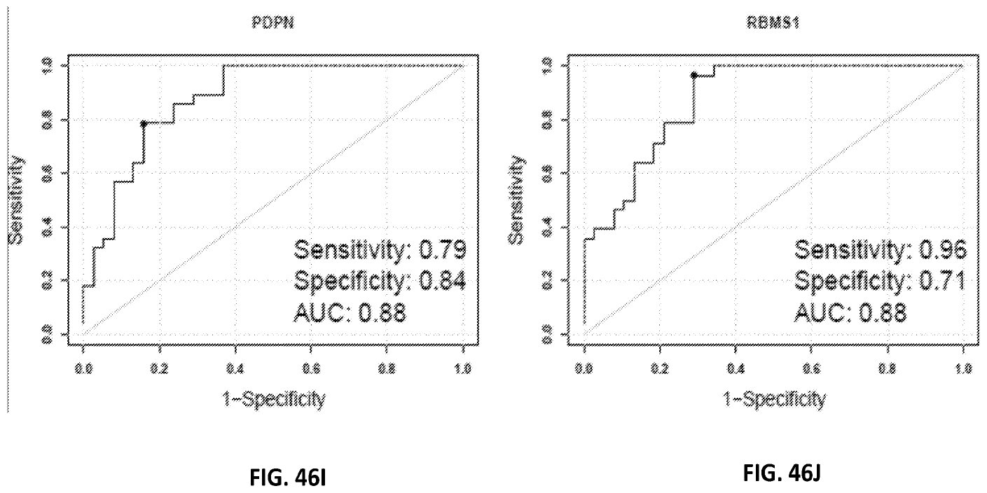

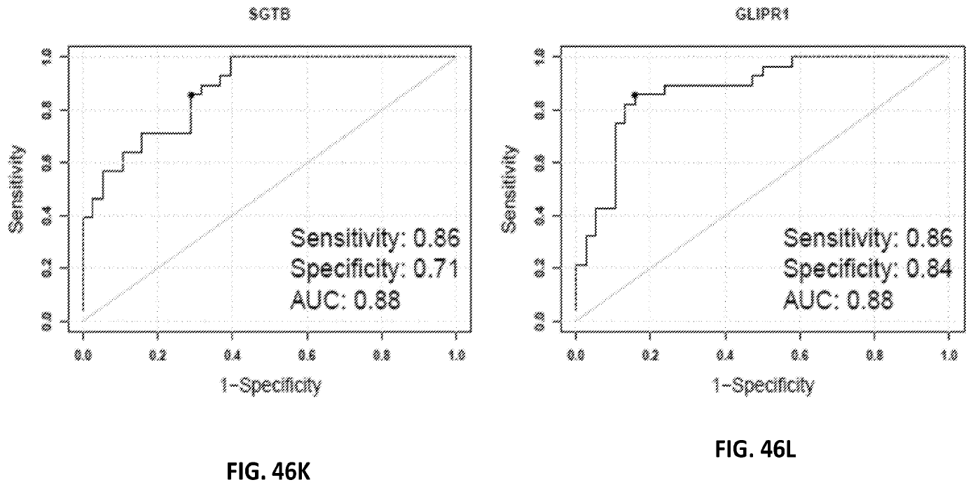

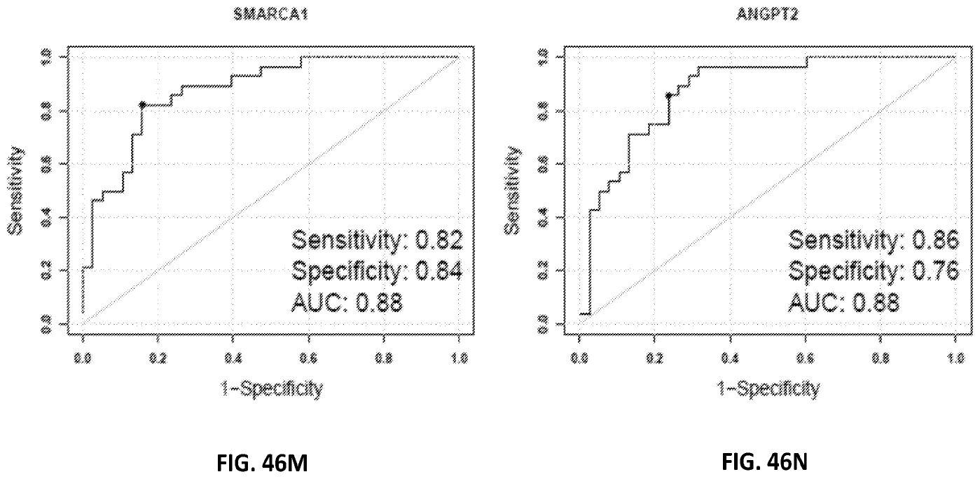

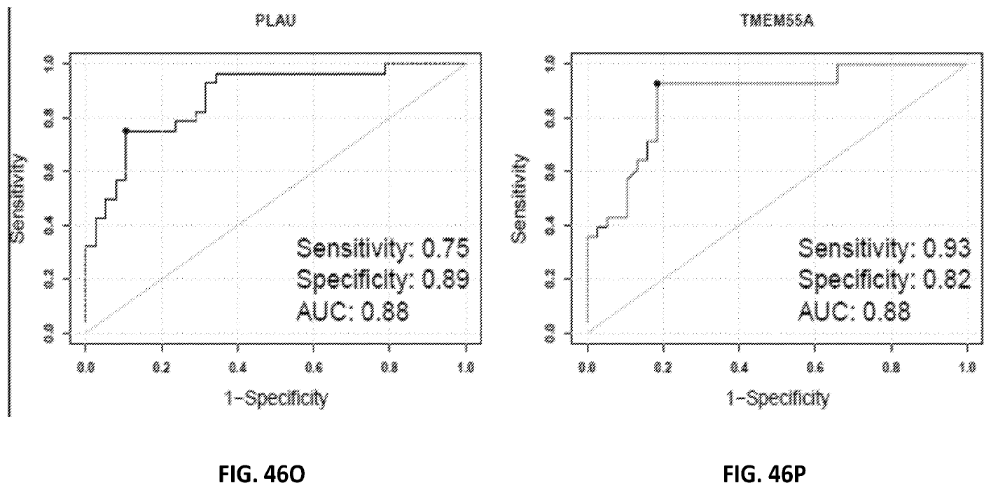

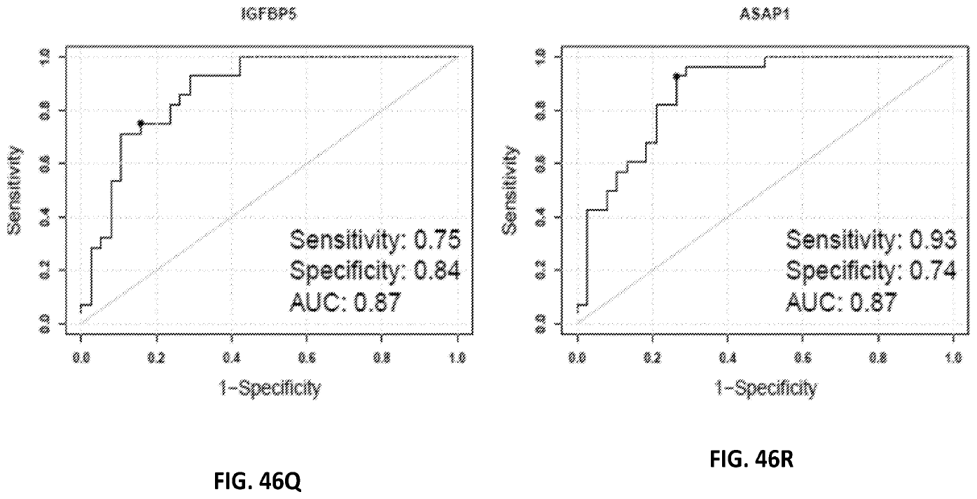

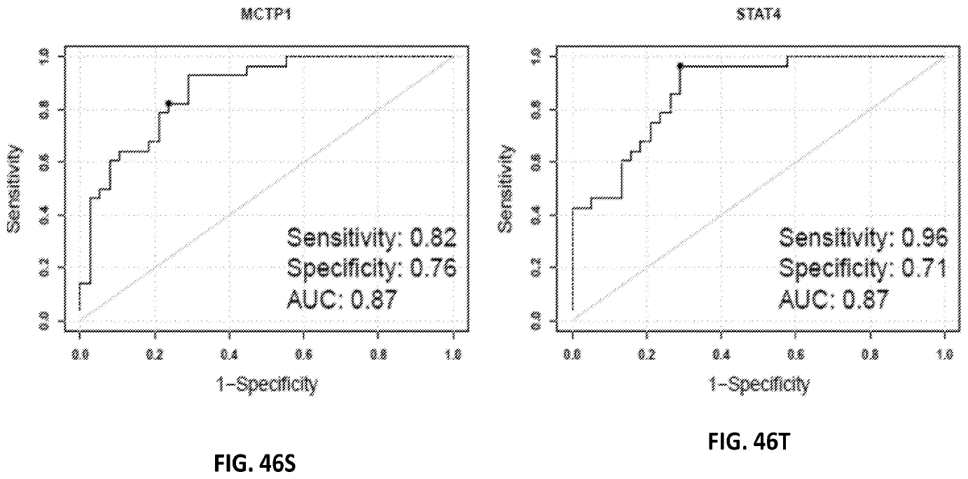

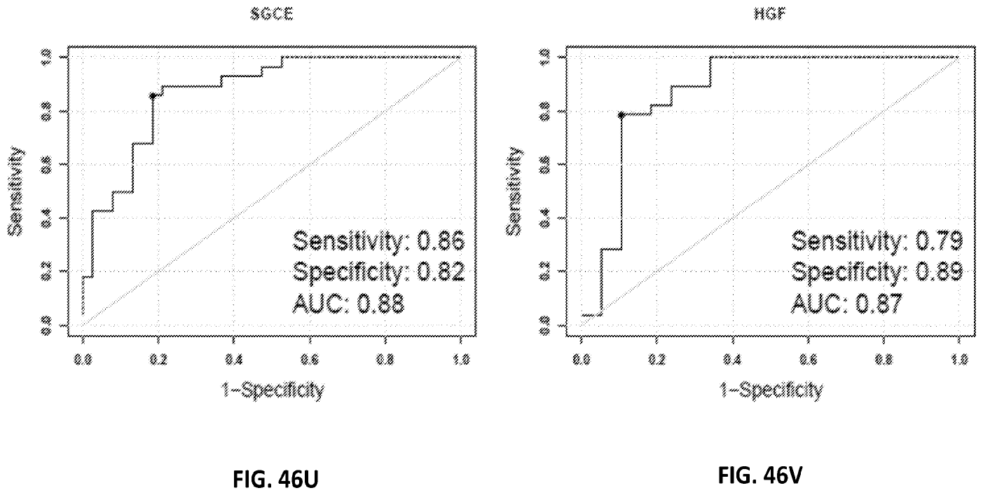

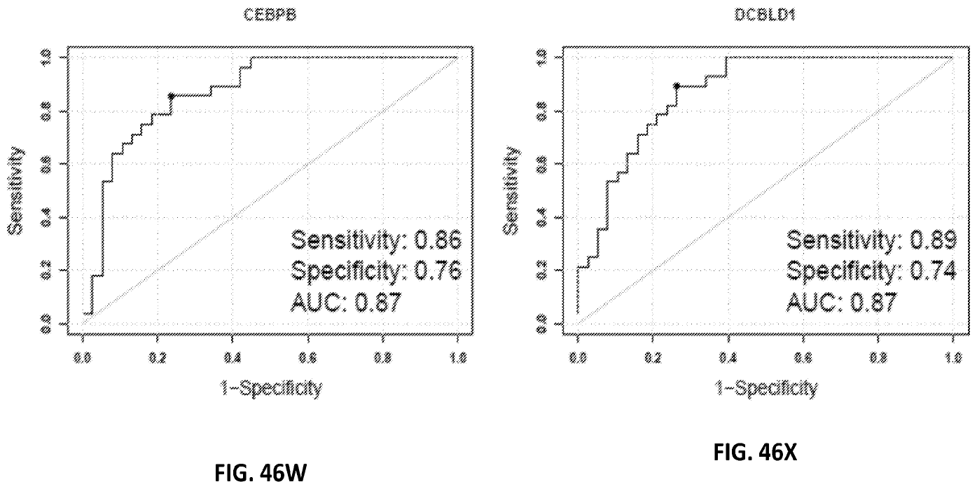

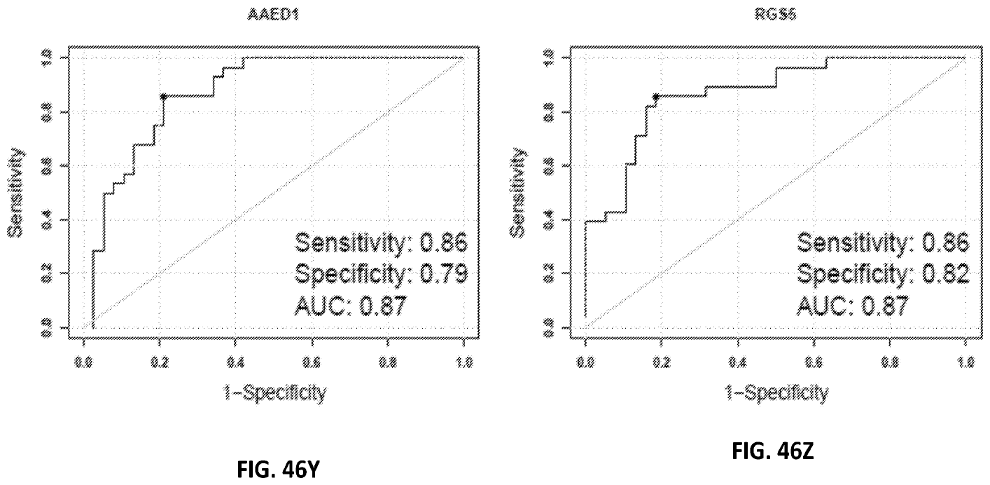

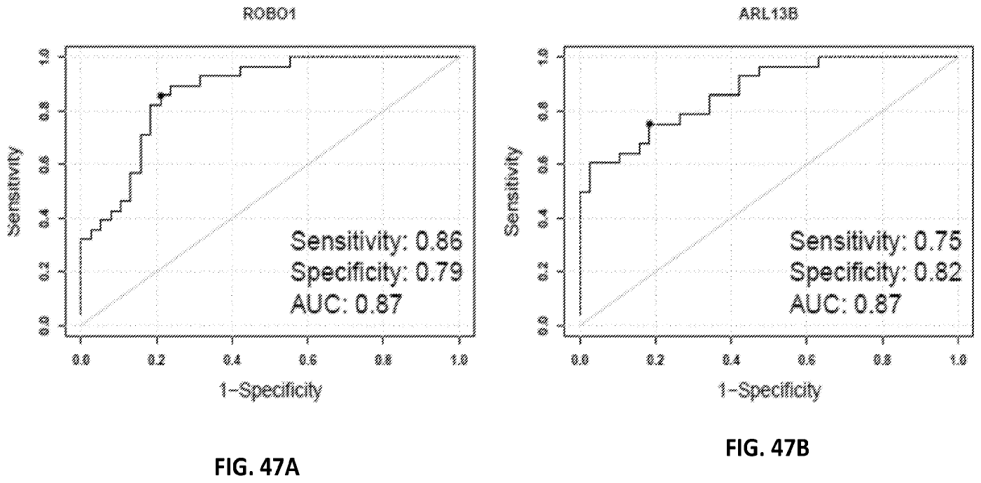

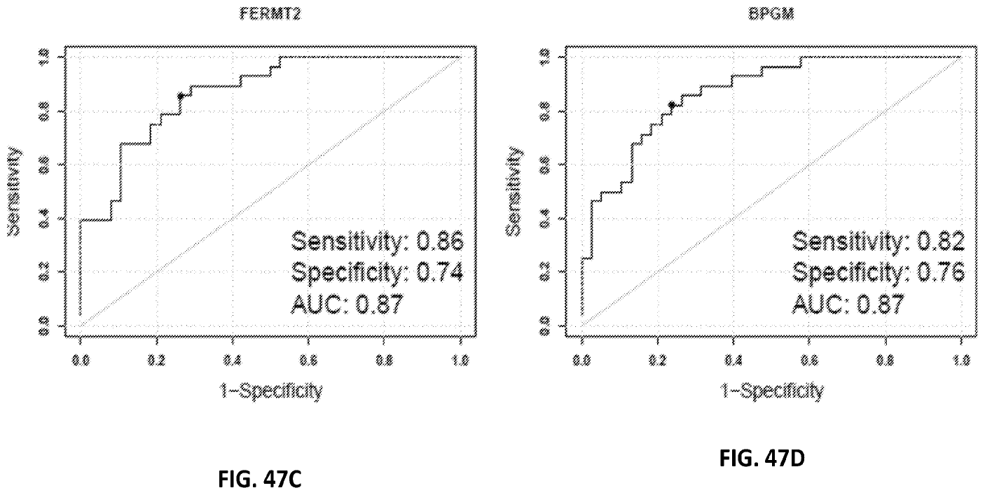

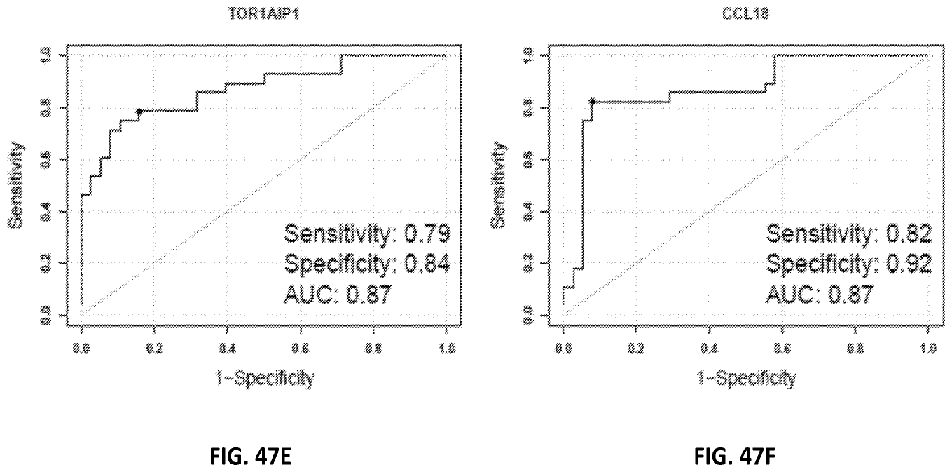

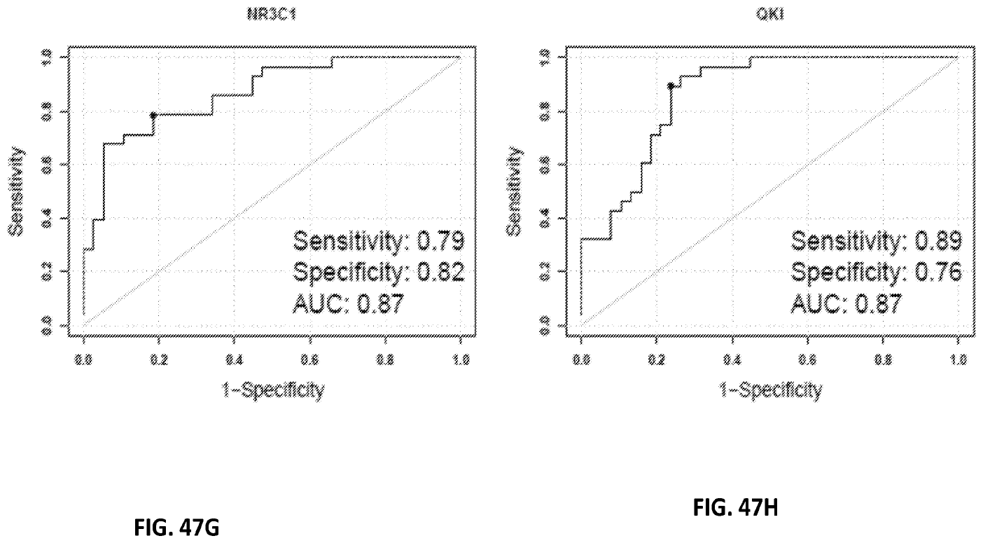

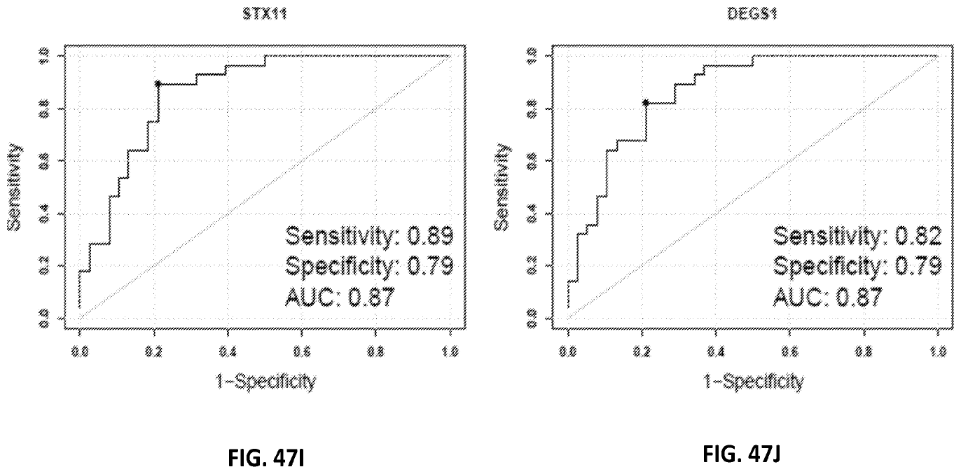

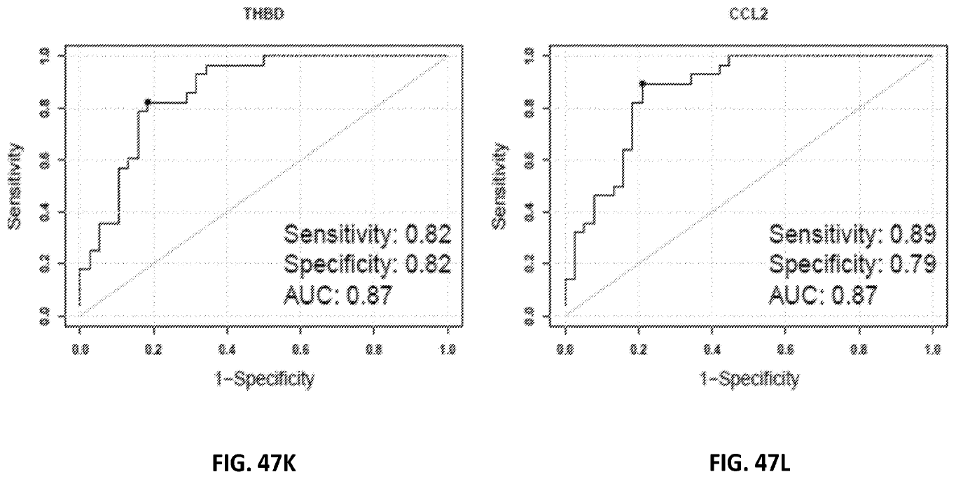

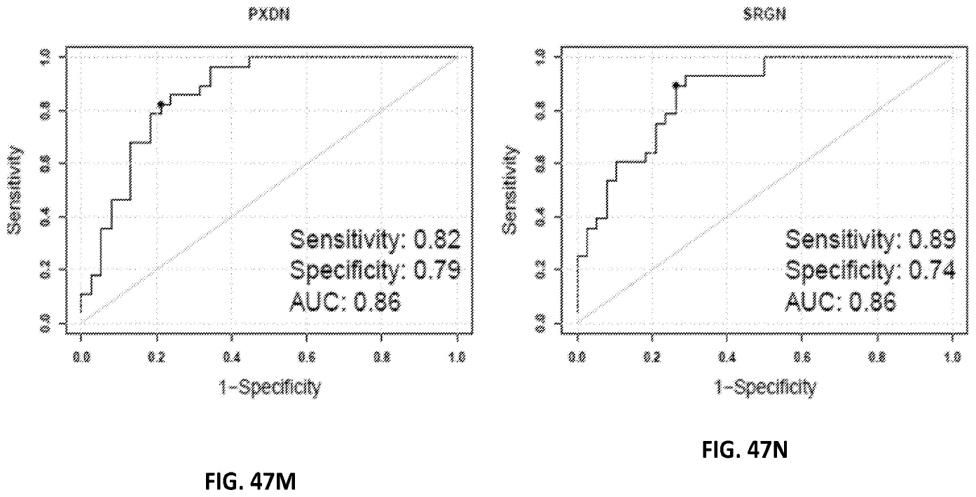

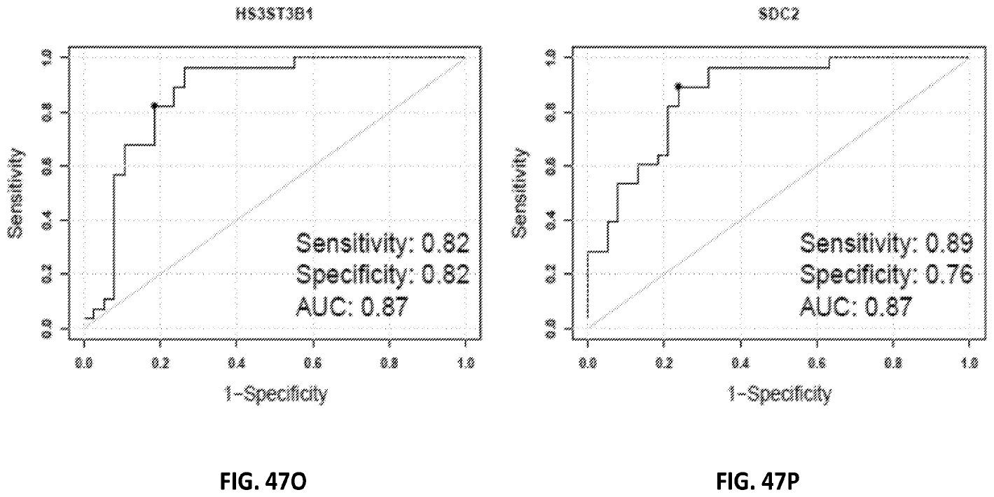

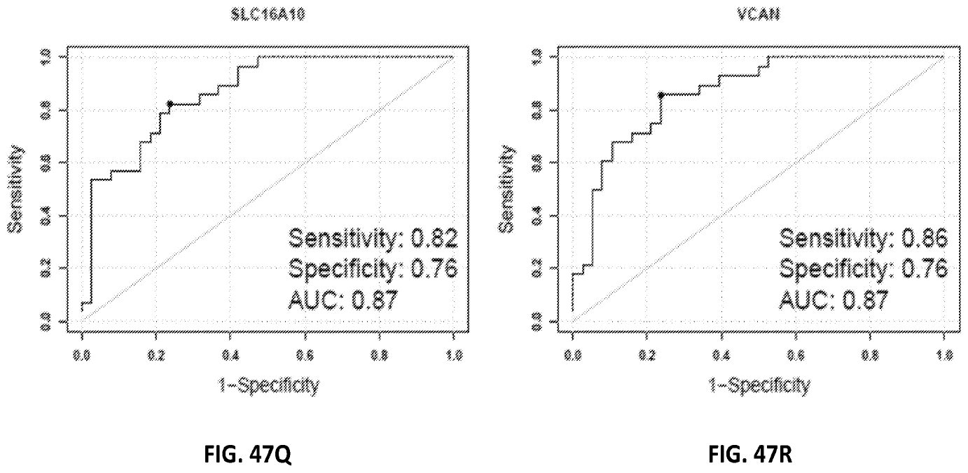

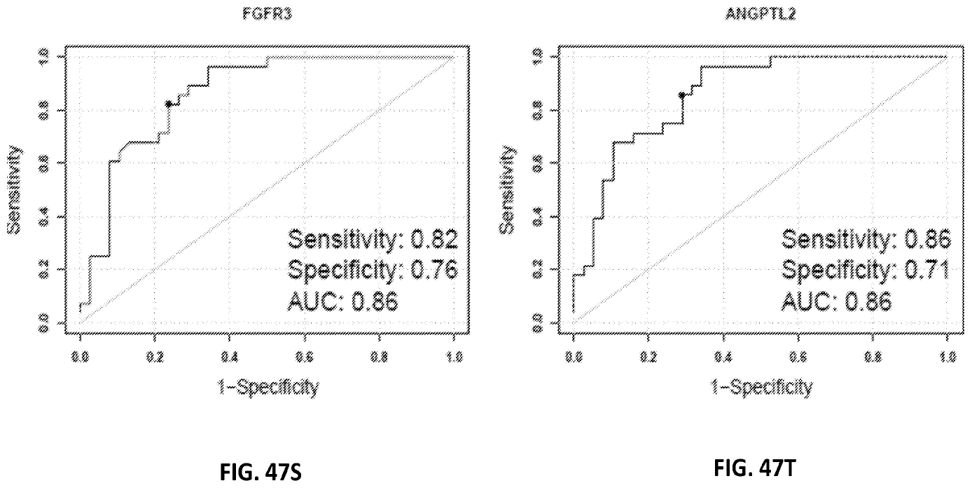

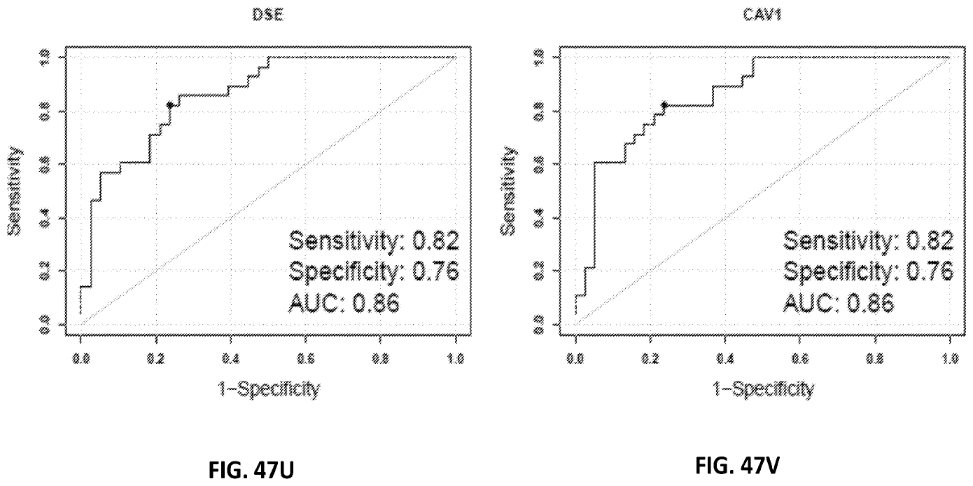

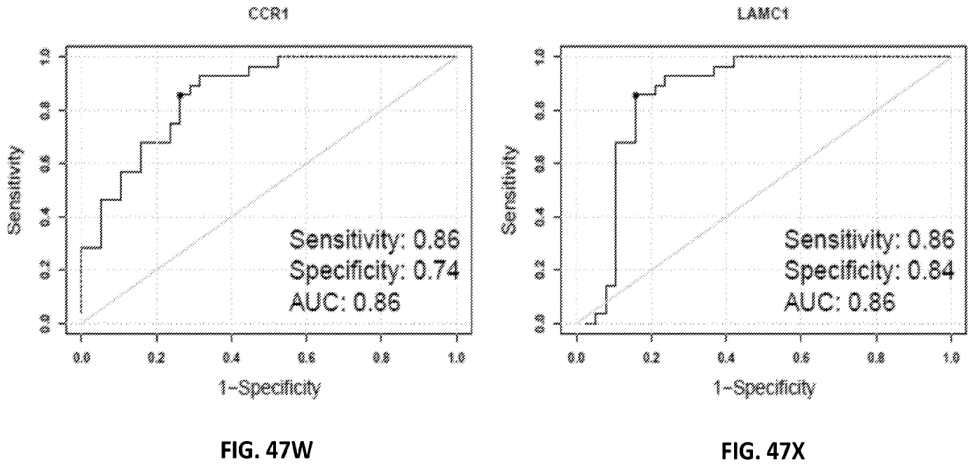

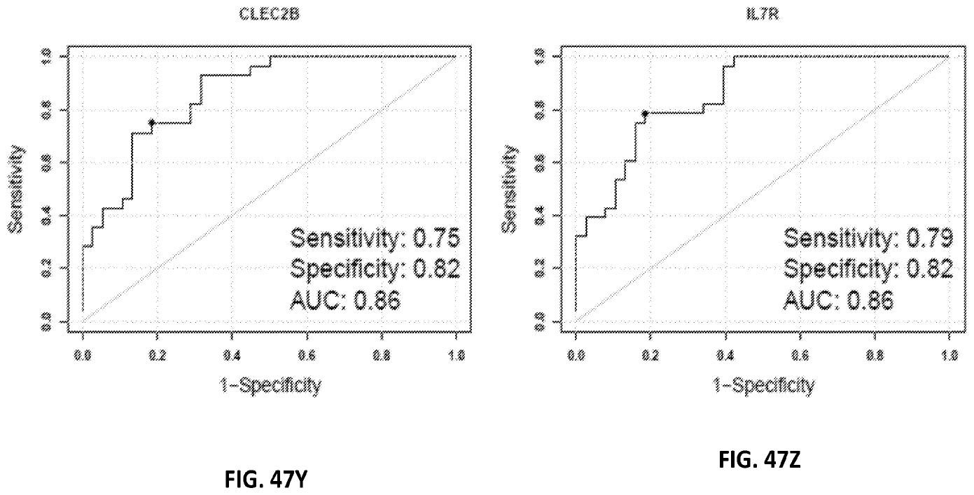

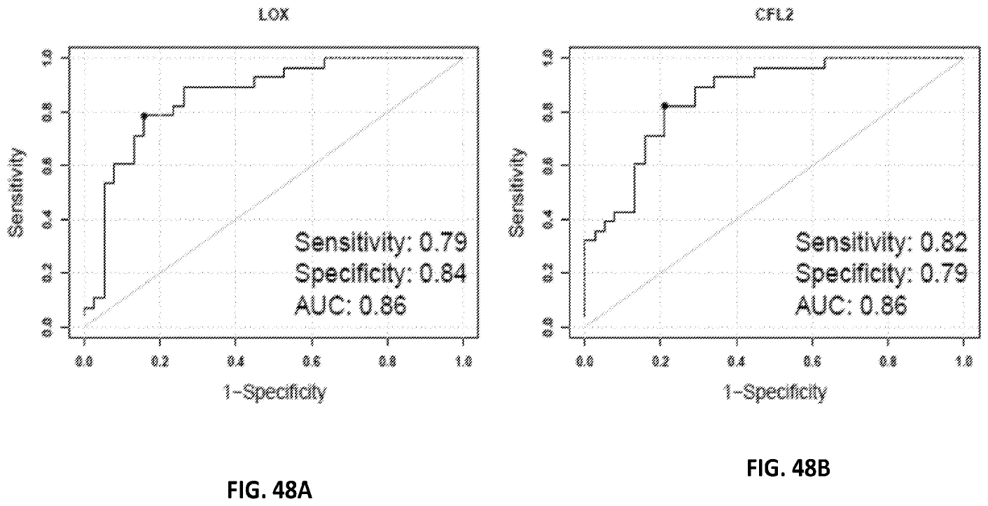

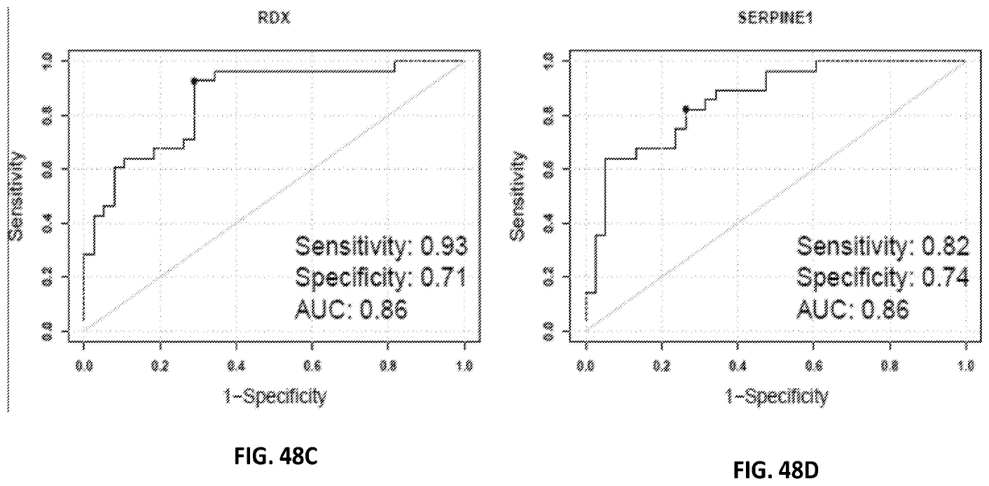

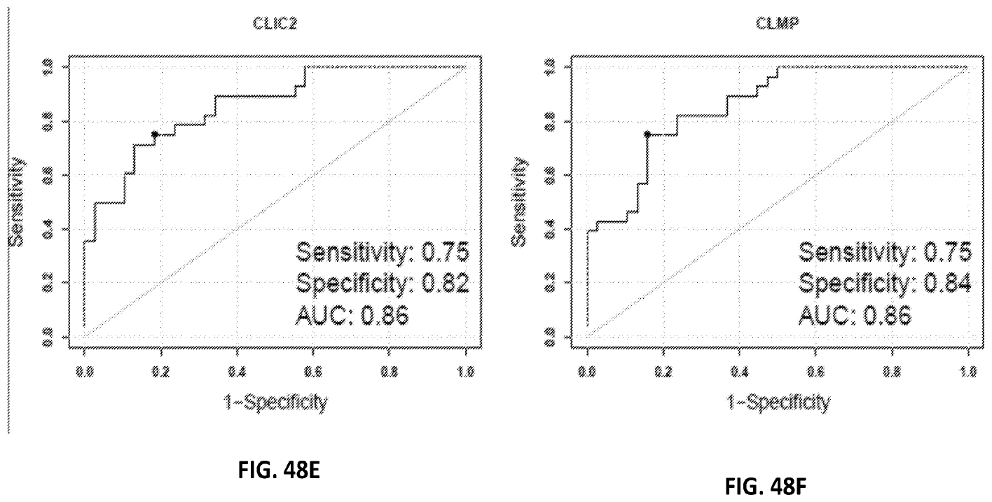

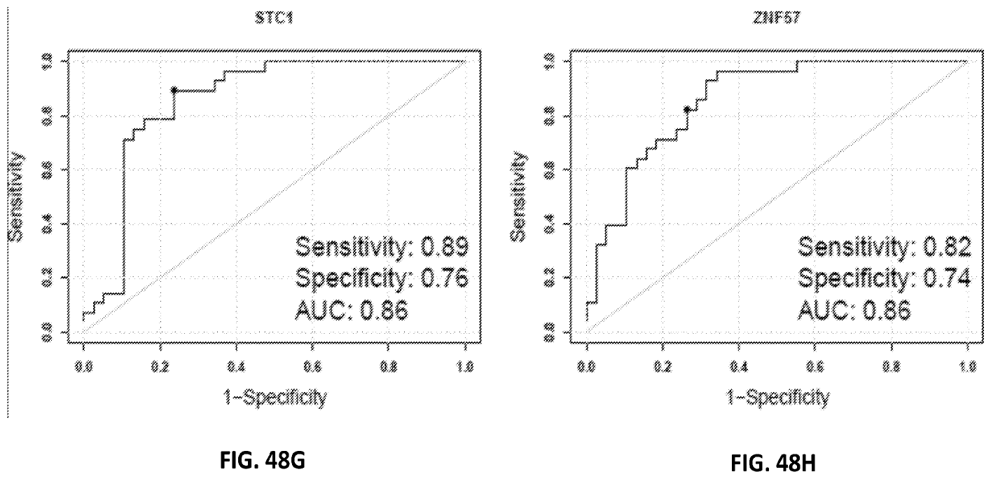

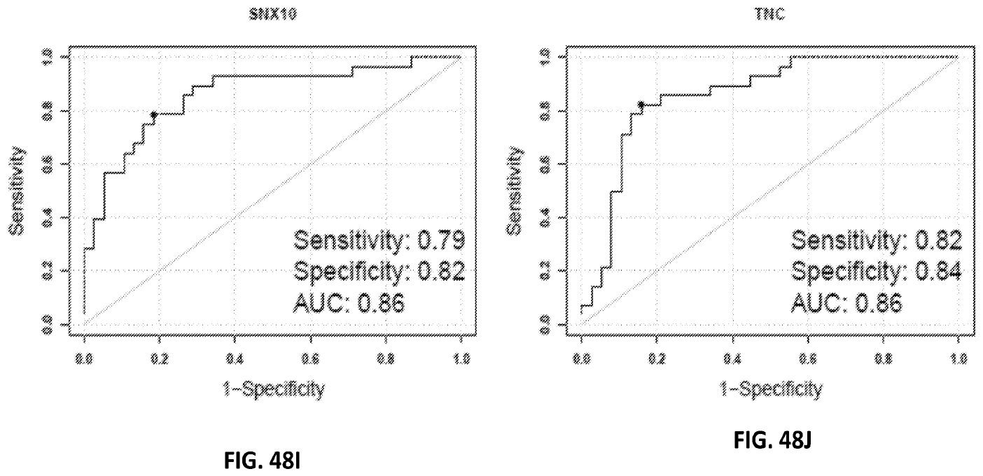

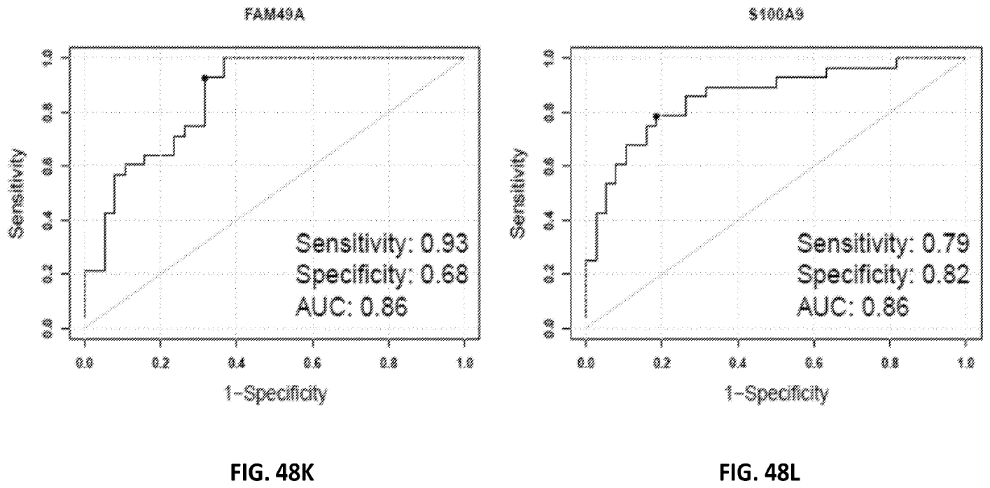

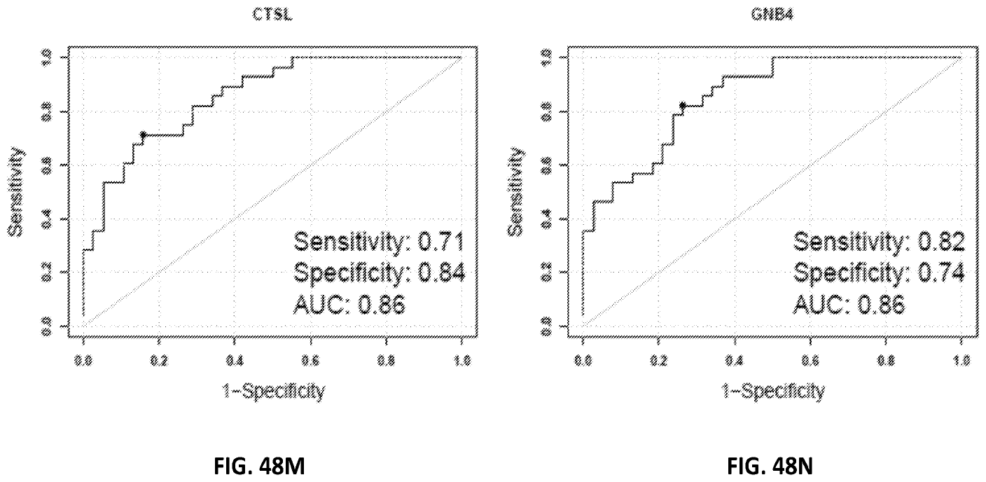

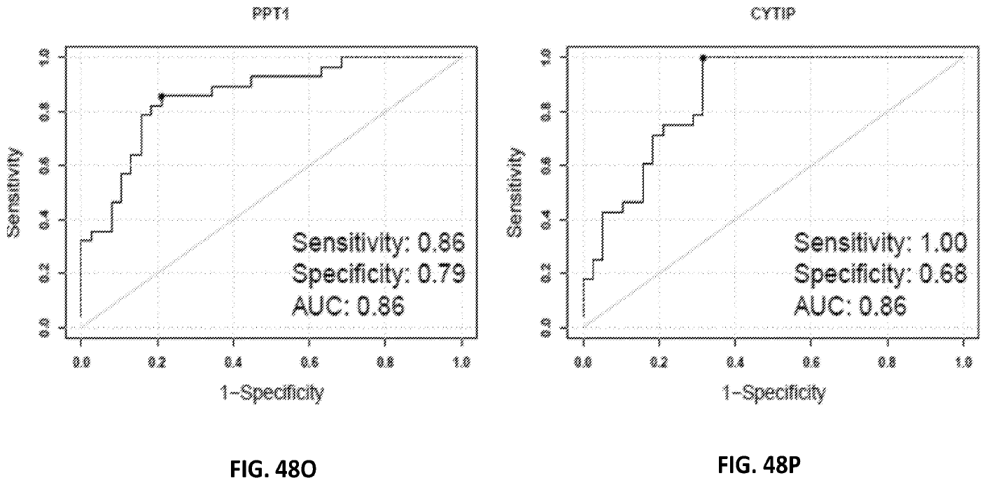

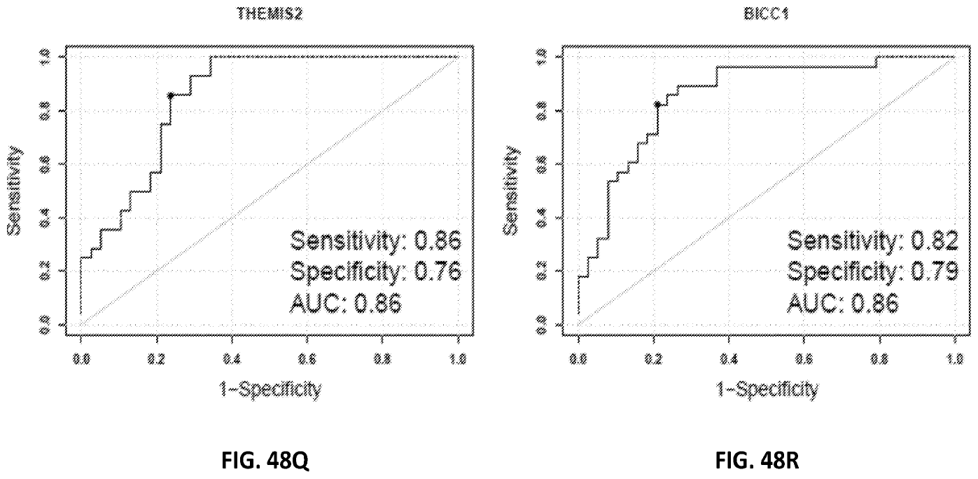

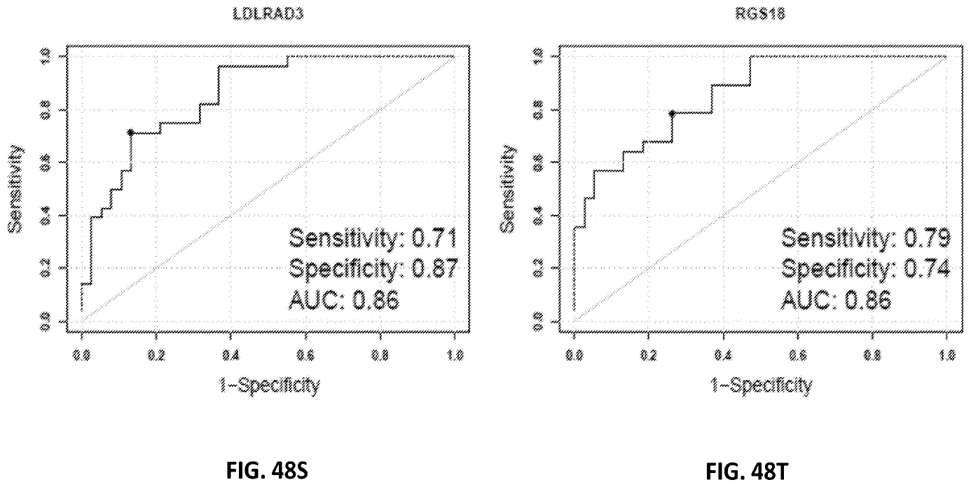

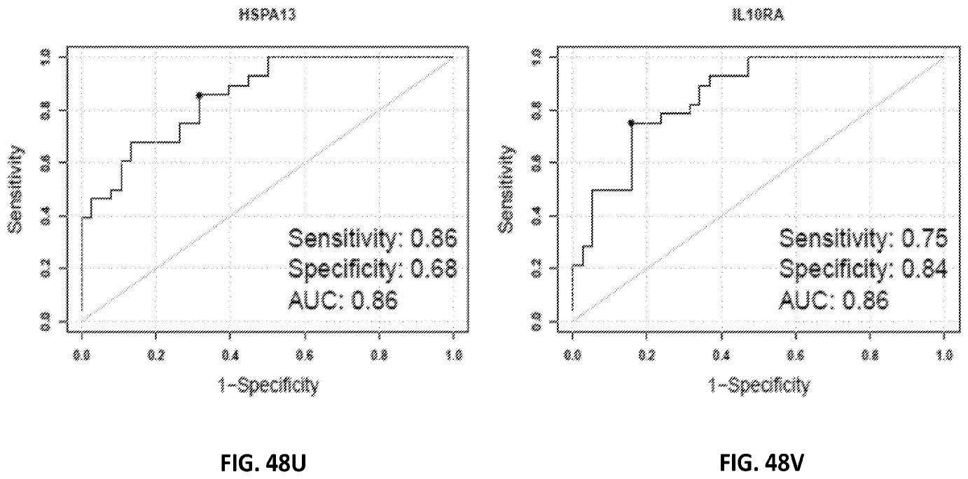

ROC analysis of each individual gene to estimate the area under the ROC (AUC) with the 95% confidence interval and the optimal cutoff point corresponding to the coordinate of (1-specificity, sensitivity) with the closest distance to the perfect classification coordinate (0,1) (i.e., 100% specificity and 100% sensitivity). The top 5 genes having AUC estimate>=0.9 are: PRNP, IL13RA2, GPX8, IKBIP, KLHL5. The boxplot of the top 100 genes with the highest AUC were drawn by response (see e.g., FIG. 41-FIG. 45). The ROC plots of the top 100 genes are drawn with the optimal cutoff points and the corresponding sensitivity and specificity at the cutoff point (see e.g., FIG. 41-FIG. 45, FIG. 46-FIG. 48).

TABLE 5
37 genes overlapped between the top 100 genes with the greatest mean
decrease gini index and the top 100 genes with the highest auc.

| | ROC analysis | | | | | RF analysis |
|---|---|---|---|---|---|---|
| GeneID | direction.1 | auc.lower | auc.est | auc.upper | cutpoint | Infliximab.non.responder.BEFORE |
| PRNP | reverse | 0.4995346 | 0.93609 | 1.3726458 | 6.987166 | 5.05E−05 |
| IL13RA2 | reverse | 0.4968169 | 0.917293 | 1.3377695 | 4.304965 | 0.000328473 |
| GPX8 | reverse | 0.4981475 | 0.912594 | 1.3270405 | 4.694742 | 0.00013953 |
| IKBIP | reverse | 0.4979802 | 0.910714 | 1.3234484 | 4.146673 | 0.000236238 |
| KLHL5 | reverse | 0.4968166 | 0.900376 | 1.3039353 | 3.928603 | 0.000206022 |
| PTX3 | reverse | 0.495632 | 0.897556 | 1.2994808 | 3.126854 | 0.000105983 |
| TXNDC15 | reverse | 0.4966156 | 0.897556 | 1.2984971 | 7.21527 | 0.000246062 |
| PDE4B | reverse | 0.4947008 | 0.890977 | 1.287254 | 5.160432 | 0.000377738 |
| C1S | reverse | 0.495512 | 0.888158 | 1.2808038 | 6.736918 | 8.48E−05 |
| TLR1 | reverse | 0.4951138 | 0.886278 | 1.2774426 | 3.68622 | 0.000229295 |
| MME | reverse | 0.4938874 | 0.885338 | 1.2767893 | 5.118889 | 0.000134266 |
| TSPAN2 | reverse | 0.4940794 | 0.884398 | 1.2747176 | 3.900095 | 0.000221704 |
| TNFRSF11B | reverse | 0.495172 | 0.881579 | 1.2679859 | 4.857184 | 0.000176777 |
| ACSL4 | reverse | 0.4936464 | 0.880639 | 1.2676318 | 5.276966 | 0.000276891 |
| CSGALNAC [symbol] | reverse | 0.4948536 | 0.880639 | 1.2664246 | 5.149009 | 8.77E−05 |
| DRAM1 | reverse | 0.4924021 | 0.880639 | 1.2688761 | 7.641144 | 0.000312285 |
| SGTB | reverse | 0.4949937 | 0.879699 | 1.2644048 | 4.008853 | 0.000262857 |
| PDPN | reverse | 0.4943981 | 0.878759 | 1.2631207 | 5.003769 | 0.000305444 |
| RBMS1 | reverse | 0.494688 | 0.878759 | 1.2628308 | 5.652724 | 0.000140236 |
| ANGPT2 | reverse | 0.493642 | 0.87594 | 1.2582377 | 3.906948 | 0.000147876 |
| TMEM55A | reverse | 0.4932275 | 0.87547 | 1.2577124 | 4.904756 | 0.000147381 |
| HGF | reverse | 0.4922317 | 0.87406 | 1.2558886 | 4.405596 | 0.000267772 |
| STAT4 | reverse | 0.4939778 | 0.87312 | 1.2522628 | 6.085051 | 4.40E−05 |
| RGS5 | reverse | 0.4932757 | 0.87218 | 1.2510852 | 6.342699 | 0.000408205 |
| ROBO1 | reverse | 0.4936238 | 0.87218 | 1.2507371 | 7.056786 | 0.000325649 |
| TOR1AIP1 | reverse | 0.492262 | 0.871241 | 1.2502192 | 6.776874 | 0.000139531 |
| CCL18 | reverse | 0.4912172 | 0.870301 | 1.2493843 | 6.605382 | 0.000389665 |
| HS3ST3B1 | reverse | 0.4914569 | 0.867481 | 1.2435055 | 4.955863 | 0.000162064 |
| SDC2 | reverse | 0.4929745 | 0.867481 | 1.2419879 | 6.186364 | 0.000182229 |
| PXDN | reverse | 0.492445 | 0.864662 | 1.2368783 | 6.462694 | 0.000180648 |
| DSE | reverse | 0.4928137 | 0.863722 | 1.2346299 | 4.961414 | 0.000248639 |
| SNX10 | reverse | 0.4905421 | 0.859962 | 1.2293827 | 8.33534 | 0.000208184 |
| TNC | reverse | 0.4909697 | 0.859962 | 1.2289551 | 5.474045 | 0.000195385 |
| CLIC2 | reverse | 0.4920492 | 0.859023 | 1.225996 | 6.080585 | 9.29E−05 |
| PPT1 | reverse | 0.4913358 | 0.858083 | 1.2248296 | 8.514224 | 0.000165087 |
| RGS18 | reverse | 0.4923489 | 0.857143 | 1.2219369 | 2.962919 | 0.000111526 |
| THEMIS2 | reverse | 0.4917869 | 0.857143 | 1.2224988 | 6.196822 | 4.87E−05 |

| | RF analysis | | |
|---|---|---|---|
| GeneID | Infliximab.responder.BEFORE | MeanDecreaseAccuracy | MeanDecreaseGini |
| PRNP | 0.000120808 | 7.24E−05 | 0.014372281 |
| IL13RA2 | 0.000287708 | 0.000291923 | 0.015419903 |
| GPX8 | 0.000241414 | 0.000193556 | 0.014819574 |
| IKBIP | 0.000445714 | 0.000312045 | 0.020901887 |
| KLHL5 | 0.000284372 | 0.00022903 | 0.015895247 |
| PTX3 | 6.60E−05 | 7.57E−05 | 0.014944879 |
| TXNDC15 | 0.000369351 | 0.000295646 | 0.017951893 |
| PDE4B | 0.000481883 | 0.000378468 | 0.025408187 |
| C1S | 0.000417063 | 0.000207365 | 0.024624994 |
| TLR1 | 0.000367677 | 0.000293535 | 0.018586578 |
| MME | 0.00015917 | 0.000144614 | 0.014390939 |
| TSPAN2 | 0.000268532 | 0.000234594 | 0.026763366 |
| TNFRSF11B | 0.000213268 | 0.000186207 | 0.017520017 |
| ACSL4 | 0.000435045 | 0.000343743 | 0.031407144 |
| CSGALNAC [symbol] | 0.000209848 | 0.000137859 | 0.015966198 |
| DRAM1 | 0.000345803 | 0.00029447 | 0.020343829 |
| SGTB | 0.000355789 | 0.000281993 | 0.021626925 |
| PDPN | 0.000389127 | 0.000332152 | 0.016599031 |
| RBMS1 | 0.000261866 | 0.000200081 | 0.016012226 |
| ANGPT2 | 0.000222222 | 0.000175955 | 0.015823085 |
| TMEM55A | 0.00032202 | 0.000226501 | 0.015617059 |
| HGF | 0.000291212 | 0.000267992 | 0.017716016 |
| STAT4 | 0.000110623 | 7.08E−05 | 0.014314783 |
| RGS5 | 0.000353333 | 0.000376005 | 0.022265256 |
| ROBO1 | 0.000162657 | 0.000253118 | 0.016461612 |
| TOR1AIP1 | 0.000254988 | 0.000186878 | 0.018388662 |
| CCL18 | 0.000398073 | 0.000380825 | 0.02336265 |
| HS3ST3B1 | 0.000203492 | 0.000165769 | 0.016209192 |
| SDC2 | 0.000199444 | 0.000190851 | 0.016823767 |
| PXDN | 0.000238042 | 0.000207628 | 0.016556338 |
| DSE | 0.000360444 | 0.000291261 | 0.017902946 |
| SNX10 | 0.000291364 | 0.000236664 | 0.01481288 |
| TNC | 0.000156154 | 0.000187909 | 0.016976604 |
| CLIC2 | 0.00015899 | 0.000118367 | 0.015377314 |
| PPT1 | 0.00024241 | 0.000189869 | 0.019086713 |
| RGS18 | 1.67E−05 | 6.33E−05 | 0.014261748 |
| THEMIS2 | 0.000404221 | 0.000175236 | 0.015188916 |

[symbol]

indicates data missing or illegible when filed

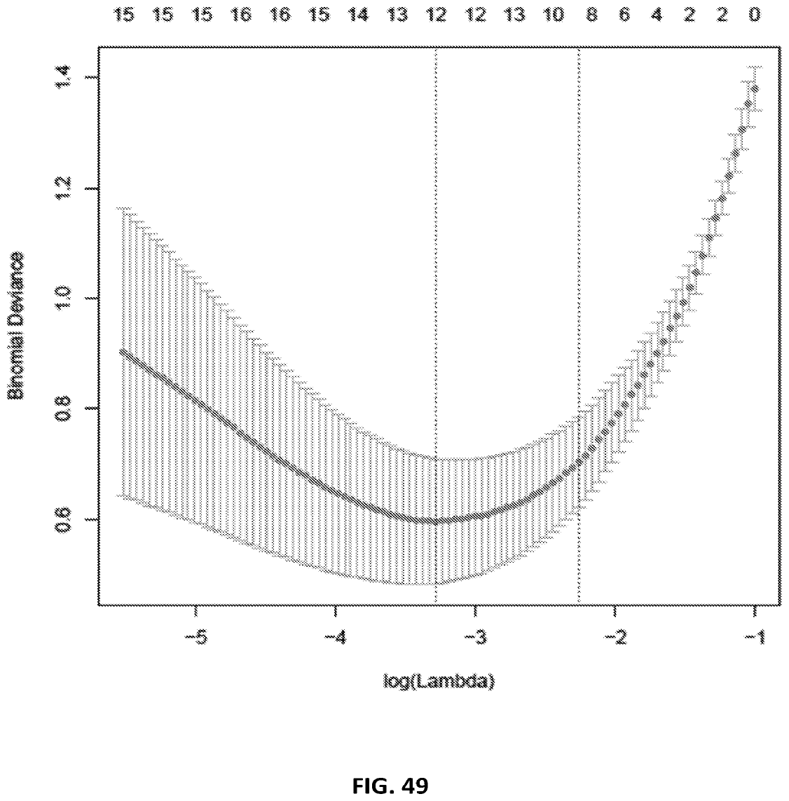

Build one tree using top 100 genes (based on mean decrease gini) from the RF analysis using R package “cart”. The lasso penalized logistic regression model with top 100 genes leading to greatest decrease in mean Gini index from the above RF analysis using R package “glmnet”. The gene expression data per gene was standardized for the penalized logistic regression model fitting. Through cross validation (CV), 9 genes were finally kept in the penalized logistic regression model at the penalty parameter lambda of 0.1042963 (the rightmost vertical line in the plot below. It is the largest penalty within 1 standard error of the optimal penalty parameter corresponding to the smallest deviance. Note the penalty parameter corresponding to the smallest CV error kept 12 genes, see e.g., FIG. 49.

TABLE 6
The coefficients of the 9 genes (and intercept) are shown below.

| Variable | coefficient |
|---|---|
| (Intercept) | 12.66847 |
| SMR3A | 1.482112 |
| DRAM1 | −0.14616 |
| SLC23A2 | −0.28982 |
| HDGFRP3 | −0.00719 |
| IL13RA2 | −0.59576 |
| GPX8 | −0.70709 |
| PRNP | −0.41885 |
| STAT4 | −0.29415 |
| HIF1A | −0.24065 |

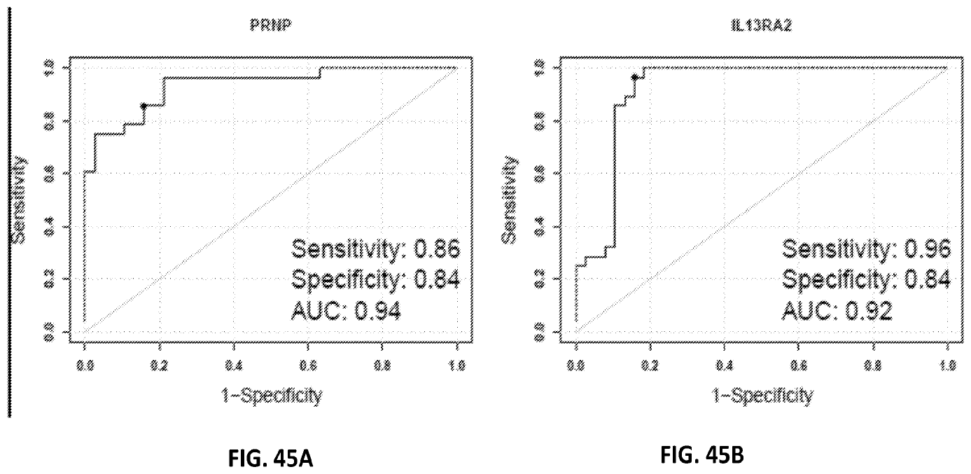

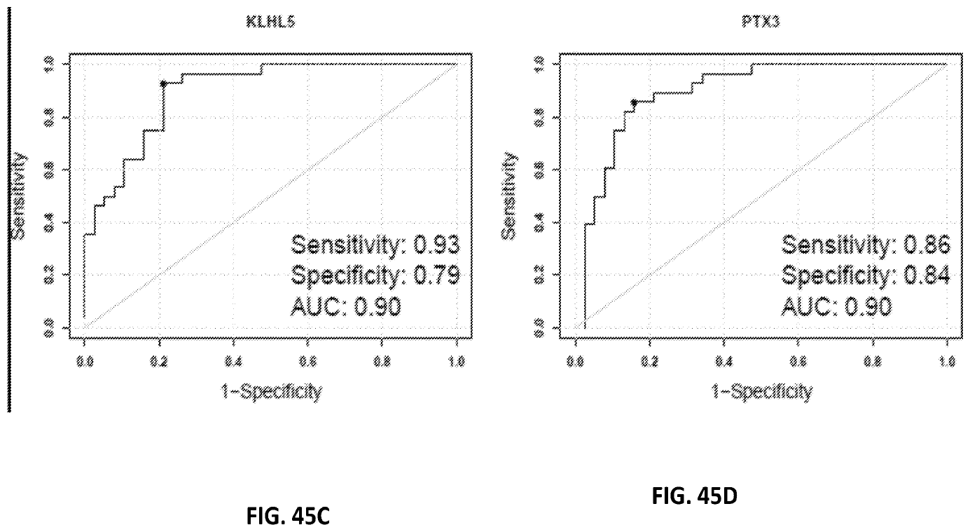

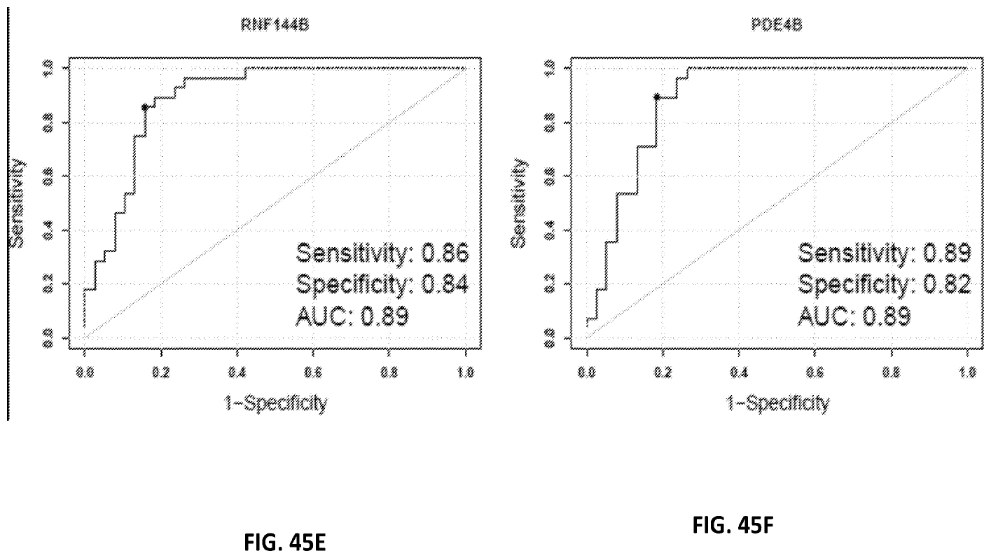

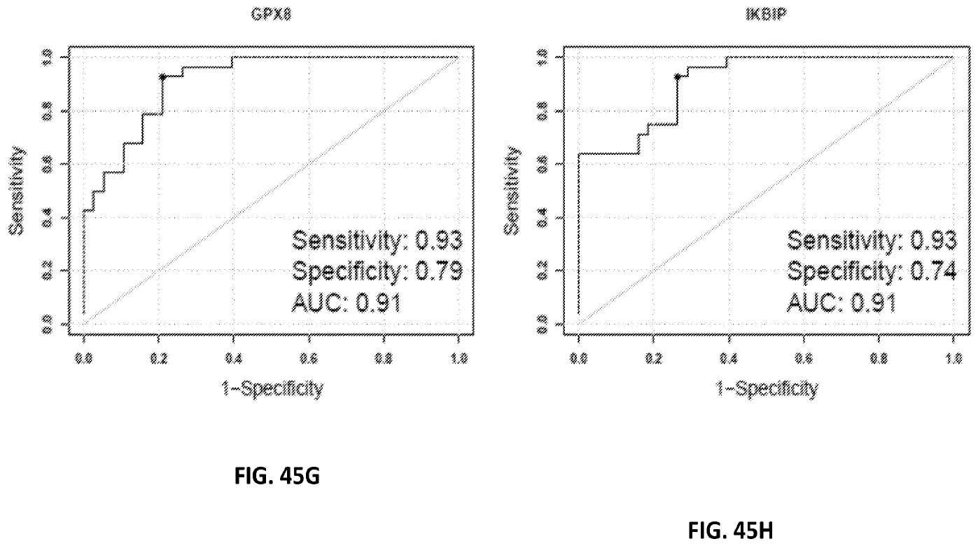

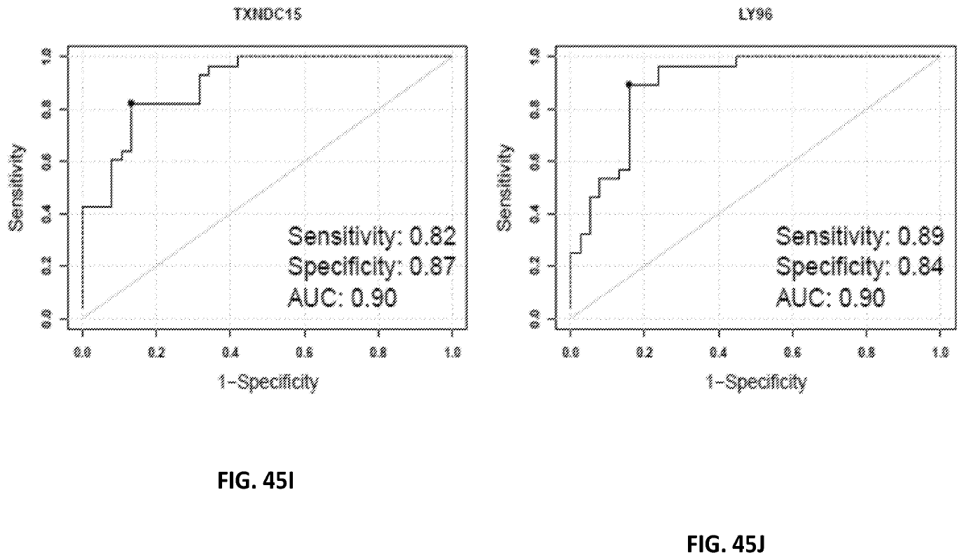

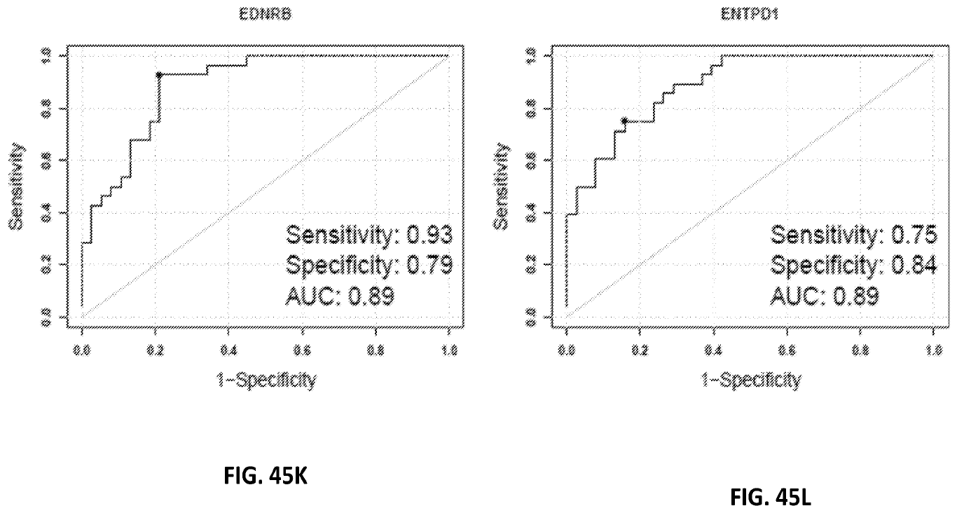

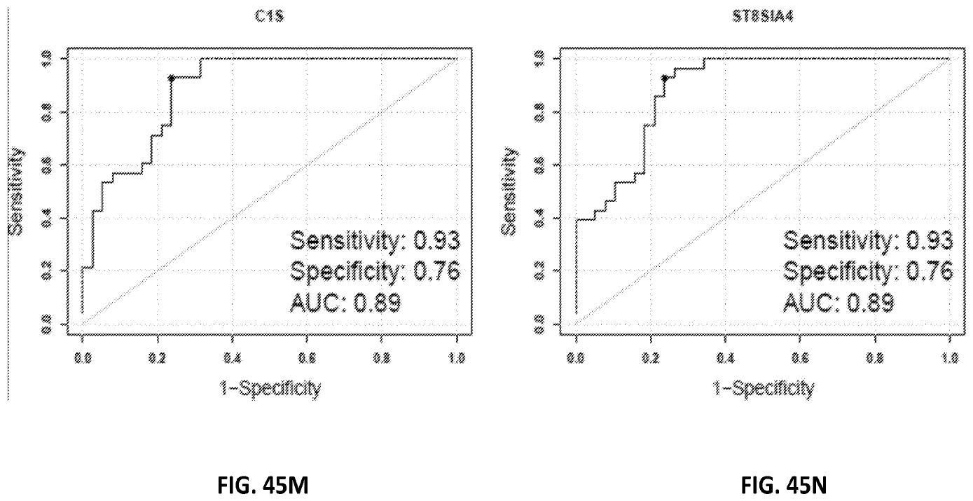

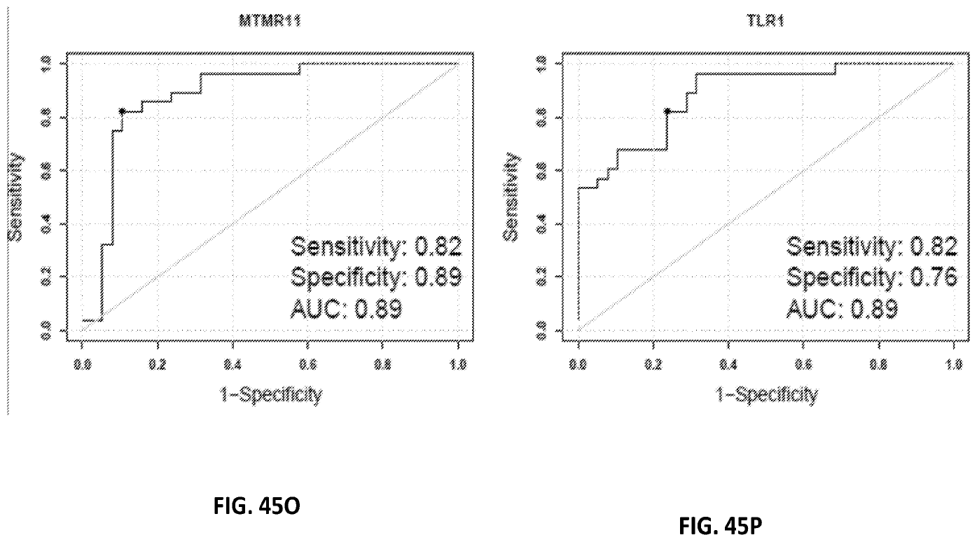

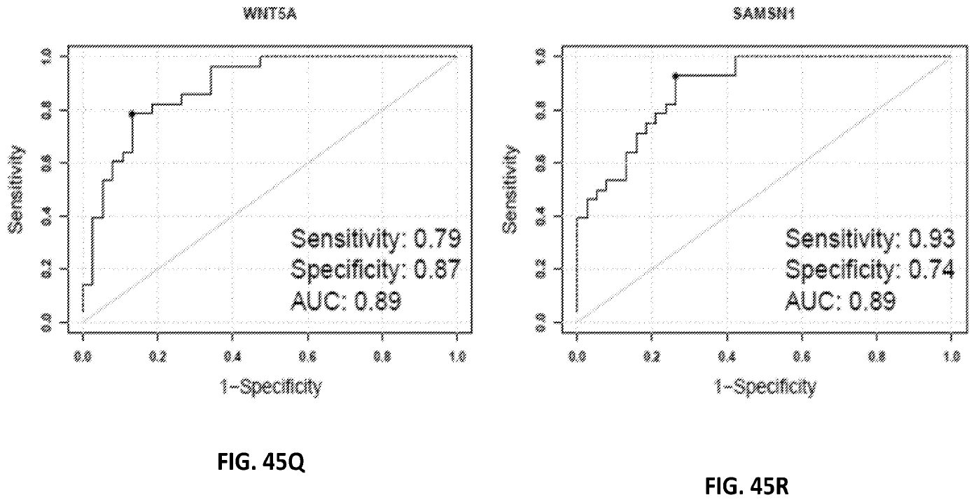

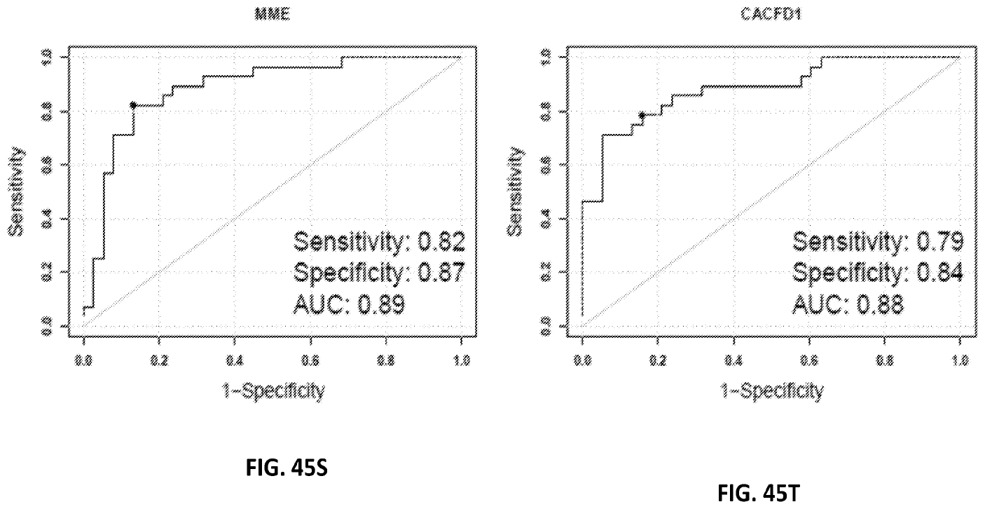

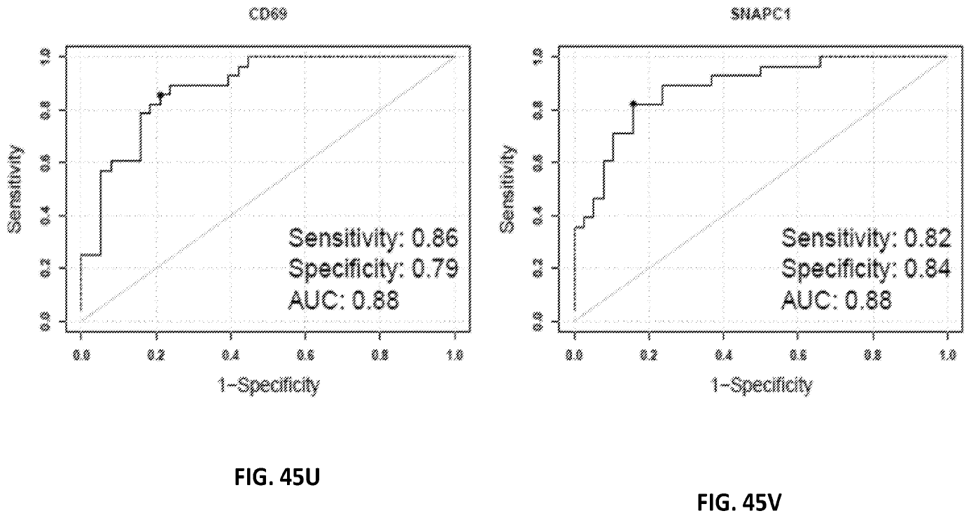

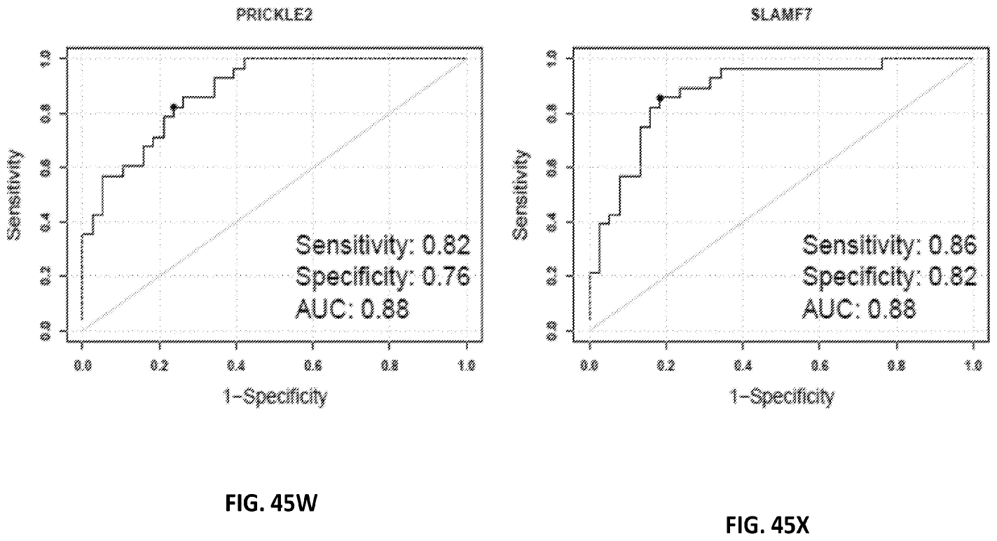

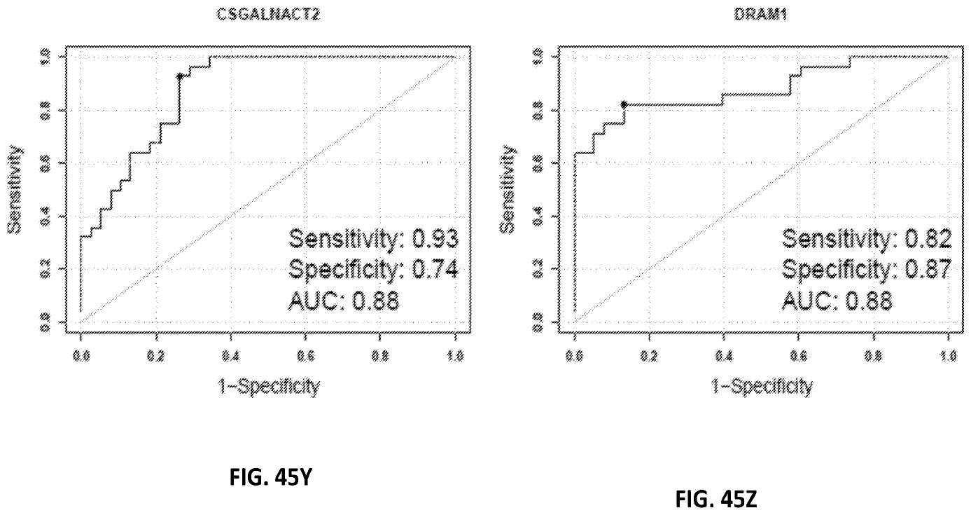

The linear predictor constructed using the penalized logistic regression model based on the 9 genes improved the AUC to 0.99, as compared with 0.93 from the best gene's AUC in the individual gene ROC analysis (see e.g., FIG. 49) and more importantly increased both sensitivity and specificity to >0.9. This model will be validated in independent cohorts. The lasso penalized logistic regression model was conducted similarly using gene expression data with the top 100 genes of the highest AUC. Also 9 genes were selected based on penalty parameter of 0.05438 (see CV plot, FIG. 45).

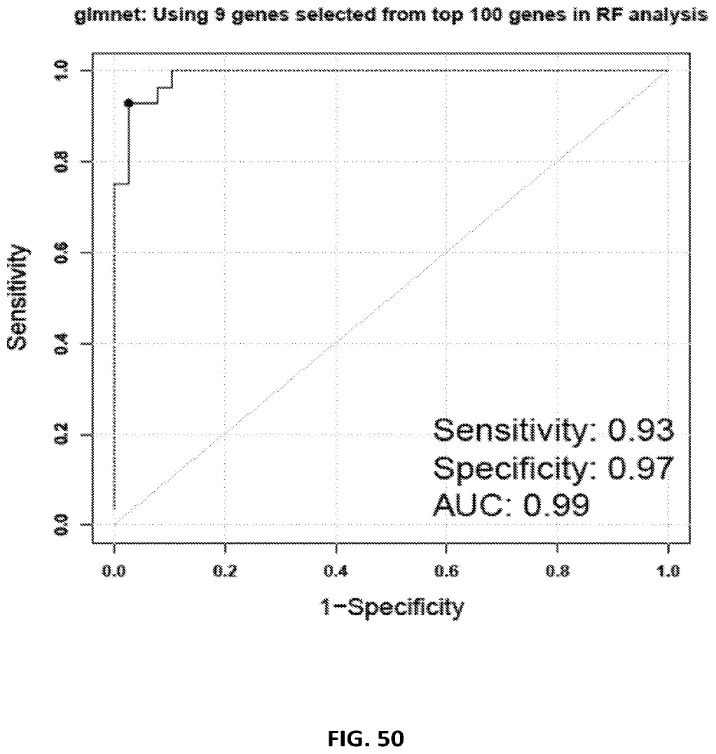

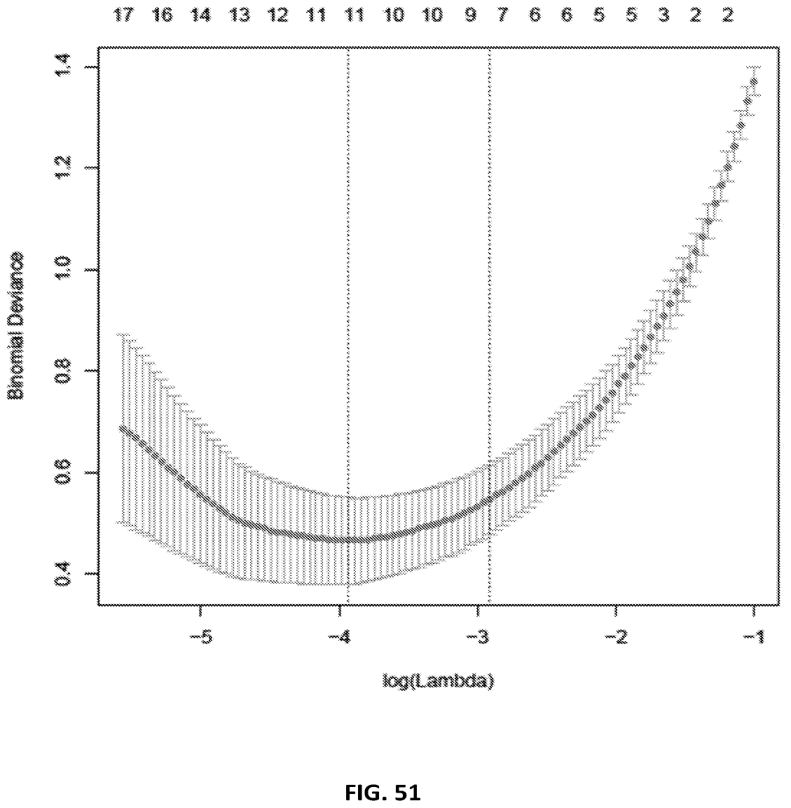

TABLE 7
Their coefficients are shown below (see e.g., FIG. 50).

| Variable | coefficient |
|---|---|
| (Intercept) | 22.97536 |
| PRNP | −0.17088 |
| IL13RA2 | −0.74211 |
| GPX8 | −1.95242 |
| DRAM1 | −0.59201 |
| STAT4 | −0.8049 |
| TOR1AIP1 | −0.05234 |
| CCL18 | −0.00022 |
| S100A9 | −0.03044 |
| ZNF57 | 0.423264 |

Notice that 5 genes (DRAM1, GPX8, IL13RA2, PRNP, STAT4) overlapped with the analyses started with the top 100 RF genes. The derived linear predictor based on these 9 genes also led to the same improvement of AUC, sensitivity, and specificity. Since the penalized logistic regression models starting with the top 100 RF genes or the top AUC genes ultimately shared 5 genes. It's suspected that maybe the use of these 5 genes is enough. The expression of the 5 genes (in original scale) are used in a logistic regression model.

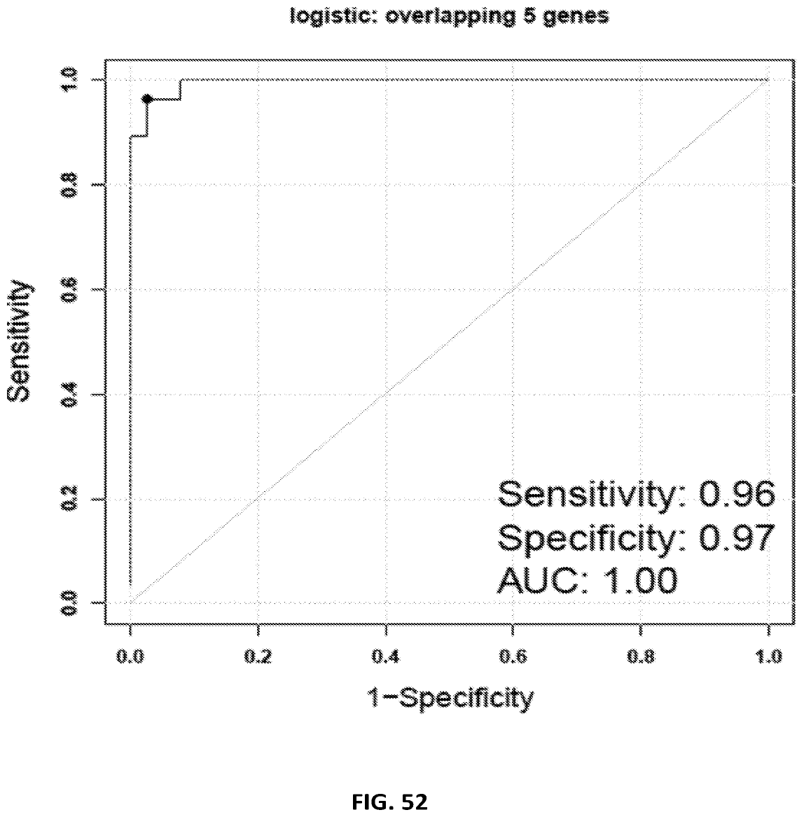

TABLE 8
The coefficients are shown below (see e.g., FIG. 52).

| Variable | coefficient |
|---|---|
| (Intercept) | 132.9813 |
| PRNP | 1.963612 |
| IL13RA2 | −2.71323 |
| GPX8 | −12.5419 |
| DRAM1 | −2.43214 |
| STAT4 | −7.03906 |

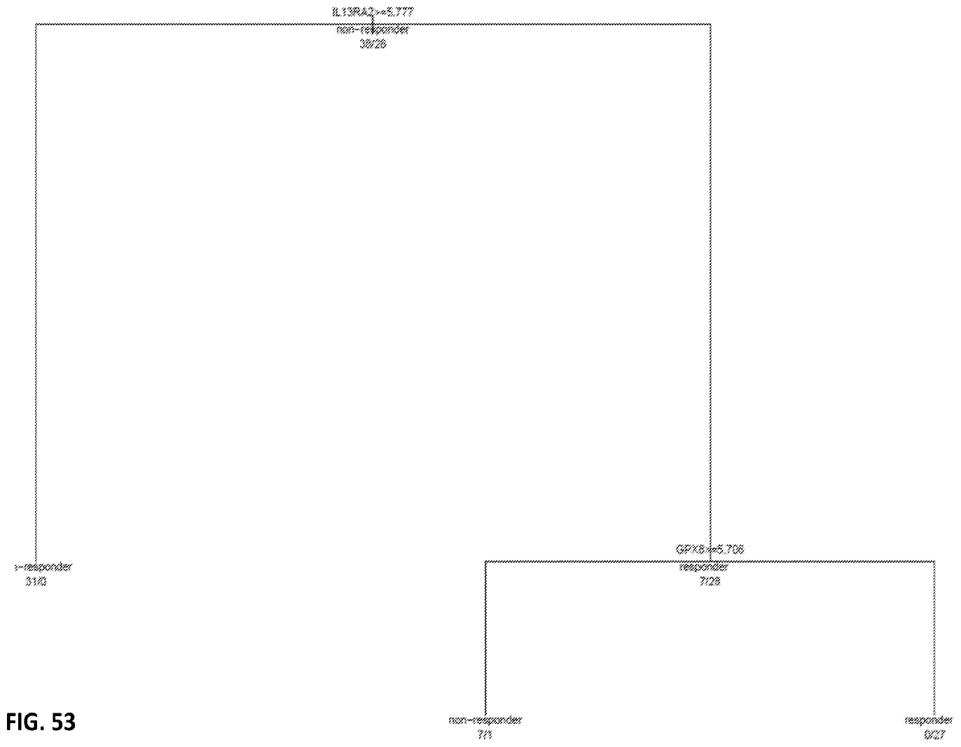

The ROC curve based on the linear predictor constructed using the 5 genes only led to an AUC of 1 and improved sensitivity to 0.96 (see e.g., FIG. 52). Finally, to provide some insight on how one tree can predict response well. The top 100 RF genes were further to build one single tree (see e.g., FIG. 53) using the R package “rpart” with the tree shown below. The single tree first split all the 66 patients (38/28 non-responder/responders) by IL13RA2 at a cutoff point of 5.777, which identified 21 non-responders with IL13RA2 above the threshold (the leftmost node). The 35 remaining patients (7 non-responders/28 responder) were split based on GPX8 by a cutoff point of 5.706. 27 out of 28 responders were identified to have GPX8<5.706 (the rightmost node). The middle node was left with 7 non-responders and 1 responder with GPX8>=5.706.

1-18. (canceled)

19. A method treating a human subject with inflammatory bowel disease (IBD) comprising:
a) identifying a human subject with IBD as a likely responder to treatment with anti-TNF or anti-α4β7 antibodies based on detected levels of PAI-1/SERPINE protein and/or mRNA in a sample from said human subject that are lower than a corresponding protein or mRNA reference value; and
b) administering an effective amount of said anti-TNF or said anti-α4β7 antibodies to said human subject.

20. The method of claim 19, wherein said human subject has detected levels of PAI-1/SERPINE protein or mRNA lower than said reference value when said detected levels of PAI-1/SERPINE protein or mRNA have a log 2 expression value relative to said reference value of about 7.5 or less.

21. The method of claim 19, wherein said human subject has detected levels of PAI-1/SERPINE protein or mRNA lower than said reference value when said detected levels of PAI-1/SERPINE protein or mRNA have a log 2 expression value relative to said reference value of about 7.4 or less.

22. The method of claim 19, wherein said human subject has detected levels of PAI-1/SERPINE protein or mRNA lower than said reference value when said detected levels of PAI-1/SERPINE protein or mRNA have a log 2 expression value relative to said reference value of about 6.5 or less.

23. The method of claim 19, further comprising: further identifying said human subject with IBD as a likely responder to said treatment with anti-TNF or anti-α4β7 antibodies based on detected levels of CCL2 in a sample from said human subject as having a log 2 expression value relative to a reference value of about 9.2 or less.

24. The method of claim 19, wherein said anti-TNF antibodies comprises infliximab.

25. The method of claim 19, wherein said anti-α4β7 antibodies comprise vedolizumab.

26. The method of claim 19, wherein said administering employs said anti-TNF antibodies.

27. The method of claim 19, wherein said administering employs said anti-α4β7 antibodies.

28. The method of claim 19, wherein said sample comprises intestinal tissue.

29. The method of claim 19, wherein said sample comprises saliva.

30. The method of claim 19, wherein said sample comprises blood.

31. The method of claim 19, wherein said sample is a plasma sample.

32. The method of claim 19, wherein said sample is a serum sample.

33. The method of claim 19, wherein said sample comprises urine.

34. A method treating a human subject with inflammatory bowel disease (IBD) comprising:
a) identifying a human subject with IBD as a having active disease IBD based on detected levels of PAI-1/SERPINE protein and/or mRNA in a sample from said human subject that are higher than a corresponding protein or mRNA reference value; and
b) administering an effective amount of an antibiotic or anti-inflammatory to said human subject.

35. The method of claim 34, wherein said human subject has detected levels of PAI-1/SERPINE protein or mRNA higher than said reference value when said detected levels of PAI-1/SERPINE protein or mRNA have a log 2 expression value relative to said reference value of more than about 7.5.

36. The method of claim 34, wherein said sample comprises intestinal tissue.

37. The method of claim 34, wherein said sample comprises saliva or urine.

38. The method of claim 34, wherein said sample is a plasma sample or a serum sample, or wherein said sample comprises blood.
